SEQUENCE LISTING (18 sequences)

SEQ ID NO: 1 - DNA, 1089 bp, Homo sapiens
  G-protein coupled receptor (GPCR) CCX-CKR2 (RDC1) coding sequence
atggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatga

SEQ ID NO: 2 - protein, 362 aa, Homo sapiens
  G-protein coupled receptor (GPCR) CCX-CKR2
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 3 - DNA, 1089 bp, Homo sapiens
  G-protein coupled receptor (GPCR) CCX-CKR2.2 coding sequence
atggatctgcacctcttcgactacgccgagccaggcaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcggcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggctgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtgtcggagacggagtactccgccttggagcaaaacgccaagtga

SEQ ID NO: 4 - protein, 362 aa, Homo sapiens
  G-protein coupled receptor (GPCR) CCX-CKR2.2
MDLHLFDYAEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFSGIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQNAK

SEQ ID NO: 5 - DNA, 1089 bp, Homo sapiens
  G-protein coupled receptor (GPCR) CCX-CKR2.3 coding sequence
atggatctgcatctcttcgactactcagagccagggaacttctcggacatcagctggccatgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcggcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattgtcgctgtcttctacttcctgctggccagagccatctcggcgtccagtgaccaggagaagcacagcagccggaagatcatcttctcctacgtggtggtcttccttgtctgctggttgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactctgccttggagcagagcaccaaatga

SEQ ID NO: 6 - protein, 362 aa, Homo sapiens
  G-protein coupled receptor (GPCR) CCX-CKR2.3
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIVAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 7 - DNA, 1089 bp, Homo sapiens; identical to SEQ ID NO: 1
  G-protein coupled receptor (GPCR) CCX-CKR2.4 coding sequence

SEQ ID NO: 8 - protein, 362 aa, Homo sapiens; identical to SEQ ID NO: 2
  G-protein coupled receptor (GPCR) CCX-CKR2.4

SEQ ID NO: 9 - DNA, 1089 bp, Homo sapiens
  G-protein coupled receptor (GPCR) CCX-CKR2.5 coding sequence
atggatctgcatctcttcgactactcagagccagggaacttctcggacatcagctggccgtgcaacagcagcgactgcatcgtggtggacacggtgatgtgtcccaacatgcccaacaaaagcgtcctgctctacacgctctccttcatttacattttcatcttcgtcatcggcatgattgccaactccgtggtggtctgggtgaatatccaggccaagaccacaggctatgacacgcactgctacatcttgaacctggccattgccgacctgtgggttgtcctcaccatcccagtctgggtggtcagtctcgtgcagcacaaccagtggcccatgggcgagctcacgtgcaaagtcacacacctcatcttctccatcaacctcttcagcagcattttcttcctcacgtgcatgagcgtggaccgctacctctccatcacctacttcaccaacacccccagcagcaggaagaagatggtacgccgtgtcgtctgcatcctggtgtggctgctggccttctgcgtgtctctgcctgacacctactacctgaagaccgtcacgtctgcgtccaacaatgagacctactgccggtccttctaccccgagcacagcatcaaggagtggctgatcggcatggagctggtctccgttgtcttgggctttgccgttcccttctccattatcgctgtcttctacttcctgctggccagagccatctcggcgtccagtgaccaggagaagcacagcagccggaagatcatcttctcctacgtggtggtcttccttgtctgctggttgccctaccacgtggcggtgctgctggacatcttctccatcctgcactacatccctttcacctgccggctggagcacgccctcttcacggccctgcatgtcacacagtgcctgtcgctggtgcactgctgcgtcaaccctgtcctctacagcttcatcaatcgcaactacaggtacgagctgatgaaggccttcatcttcaagtactcggccaaaacagggctcaccaagctcatcgatgcctccagagtctcagagacggagtactccgccttggagcagagcaccaaatga

SEQ ID NO: 10 - protein, 362 aa, Homo sapiens [sequence omitted]

SEQ ID NO: 11 - DNA, 21 bp, Artificial Sequence
  Description of Artificial Sequencesmall interfering RNA siRNA #1
gccgttcccttctccattatt

SEQ ID NO: 12 - DNA, 21 bp, Artificial Sequence
  Description of Artificial Sequencesmall interfering RNA siRNA #2
gagctcacgtgcaaagtcatt

SEQ ID NO: 13 - DNA, 21 bp, Artificial Sequence
  Description of Artificial Sequencesmall interfering RNA siRNA #3
gacatcagctggccatgcatt

SEQ ID NO: 14 - DNA, 50 bp, Artificial Sequence
  Description of Artificial Sequencehairpin small interfering RNA (siRNA)
caccgcctaacaagaacgtgcttctcgaaagaagcacgttcttgttaggc

SEQ ID NO: 15 - DNA, 50 bp, Artificial Sequence
  Description of Artificial Sequencehairpin small interfering RNA (siRNA)
caccgggtgaatatccaggctaagacgaatcttagcctggatattcaccc

SEQ ID NO: 16 - DNA, 50 bp, Artificial Sequence
  Description of Artificial Sequencehairpin small interfering RNA (siRNA)
caccggtcagtctcgtgcagcataacgaattatgctgcacgagactgacc

SEQ ID NO: 17 - DNA, 50 bp, Artificial Sequence
  Description of Artificial Sequencehairpin small interfering RNA (siRNA)
caccgcttccaacaatgagacctaccgaagtaggtctcattgttggaagc

SEQ ID NO: 18 - DNA, 50 bp, Artificial Sequence
  Description of Artificial Sequencehairpin small interfering RNA (siRNA)
caccgctggagaatgtgctctttaccgaagtaaagagcacattctccagc

(19) United States
(12) Patent Application Publication
(10) Pub. No.: US 2005/0214287 A1
(43) Pub. Date: Sep. 29, 2005

(54) Methods and compositions for modulating angiogenesis

(75) Inventors: Jennifer Burns, Kimberton, PA (US); Bretton Summers, Redwood City, CA (US); Yu Wang, Redwood City, CA (US); Maureen Howard, Los Altos, CA (US); Thomas Schall, Palo Alto, CA (US); Zhenhua Miao, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: ChemoCentryx, Inc., Mountain View, CA (US)

(21) Appl. No.: 11/050,345

(22) Filed: Feb. 2, 2005

Related U.S. Application Data
(60) Provisional application No. 60/541,849, filed on Feb. 3, 2004.
(60) Provisional application No. 60/598,958, filed on Aug. 4, 2004.
(60) Provisional application No. 60/626,195, filed on Nov. 8, 2004.

(51) Int. Cl. A61K 39/395; A61K 38/17

(52) U.S. Cl. 424/144.1; 514/12

(57) ABSTRACT

The present invention provides method and compositions for modulating angiogenesis.

### CROSS-REFERENCE TO RELATED PATENT APPLICATIONS

The present patent application claims benefit of priority to U.S. Provisional Patent Application No. 60/541,849, filed Feb. 3, 2004;U.S. Provisional Patent Application No. 60/598,958, filed Aug. 4, 2004; and U.S. Provisional Patent Application No. 60/626,195, filed Nov. 8, 2004, each of which are incorporated by reference for all purposes.

### BACKGROUND OF THE INVENTION

Angiogenesis is the fundamental process by which new blood vessels are formed and is essential to a variety of normal body activities (such as reproduction, development and wound repair). Although the process is not completely understood, it is believed to involve a complex interplay of molecules that both stimulate and inhibit the growth of endothelial cells, the primary cells of the capillary blood vessels. Under normal conditions these molecules appear to maintain the microvasculature in a quiescent state (i.e., one of no capillary growth) for prolonged periods that may last for weeks, or in some cases, decades. However, when necessary, such as during wound repair, these same cells can undergo rapid proliferation and turnover within as little as five days.

Although angiogenesis is a highly regulated process under normal conditions, many diseases (characterized as “angiogenic diseases”) are driven by persistent unregulated angiogenesis. Otherwise stated, unregulated angiogenesis may either cause a particular disease directly or exacerbate an existing pathological condition. Both the growth and metastasis of solid tumors are angiogenesis-dependent (Folkman, 1986, J. Cancer Res. 46:467-473; Folkman, J. Nat. Cancer Inst. 82:4-6 (1989); Folkman et al., “Tumor Angiogenesis,” Chapter 10, pp. 206-32, in THE MOLECULAR BASIS OF CANCER, Mendelsohn et al., eds. (1995).

When used as drugs in tumor-bearing animals, natural inhibitors of angiogenesis can prevent the growth of small tumors (O'Reilly et al., Cell 79:315-328 (1994)). Indeed, in some protocols, the application of such inhibitors leads to tumor regression and dormancy even after cessation of treatment (O'Reilly et al., Cell 88:277-285 (1997)). Moreover, supplying inhibitors of angiogenesis to certain tumors can potentiate their response to other therapeutic regimens (e.g., chemotherapy) (see, e.g., Teischer et al., Int. J. Cancer 57:920-925 (1994)).

Angiogenesis also plays a critical role in various biological processes such as wound healing, embryological development, the menstrual cycle, and inflammation and the pathogenesis of various diseases such as cancer, diabetic retinopathy, and rheumatoid arthritis, as described, e.g., in Folkman et al., Science 235: 442-447 (1987). Ocular neovascularization has been implicated as the most common cause of blindness and underlies the pathology of approximately twenty diseases of the eye. In certain previously existing conditions such as arthritis, newly formed capillary blood vessels invade the joints and destroy cartilage. In diabetes, new capillaries formed in the retina invade the vitreous humor, causing bleeding and blindness.

On the other hand, promoting angiogenesis in some circumstances can be beneficial. For example, promotion of angiogenesis can aid in accelerating various physiological processes and treatment of diseases requiring increased vascularization such as the healing of wounds, fractures, and burns, inflammatory diseases, ischeric heart and peripheral vascular diseases, and myocardial infarction. Inhibition of angiogenesis can aid in the treatment of diseases such as cancer, diabetic retinopathy, and rheumatoid arthritis, where increased vascularization contributes toward the progression of such diseases.

Accordingly, manipulation of angiogenesis represents a therapeutic approach by which to treat or prevent various conditions or diseases involving angiogenesis.

### BRIEF SUMMARY OF THE INVENTION

The present invention provides methods of modulating angiogenesis in a subject. In some embodiments, the methods comprise administering to the subject an agent that modulates CCX-CKR2 activity. In some embodiments, the agent modulates binding of a ligand to CCX-CKR2. In some embodiments, the ligand is selected from the group consisting of SDF-1 and 1-TAC. In some embodiments, the subject is in need of increased or decreased angiogenesis.

In some embodiments, the method promotes CCX-CKR2 activity, thereby promoting angiogenesis. In some embodiments, the agent is administered in combination with a second agent that promotes angiogenesis.

In some embodiments, the agent is a CCX-CKR2 agonist. In some embodiments, the agonist is selected from a polypeptide, an antibody and an agent with a mass of less than 1,500 daltons. In some embodiments, the CCX-CKR2 activity is promoted by expressing recombinant CCX-CK2 in a cell of the subject. In some embodiments, the cell is an endothelial cell.

In some embodiments, the CCX-CKR2 activity is promoted by administering I-TAC to the subject.

In some embodiments, I-TAC is administered locally to the subject.

In some embodiments, the subject is in need of increased vascularization. In some embodiments, the subject has a wound, fracture, burn, inflammatory disease, heart disease, restinosis, ischeric heart, peripheral vascular disease, myocardial infarction, stroke, infertility, psoriasis or scleroderma.

In some embodiments, the subject has a wound and the agent is applied to the wound, thereby enhancing wound healing. In some embodiments, the agent inhibits CCX-CKR2 activity. In some embodiments, the agent enhances CCX-CKR2 activity.

In some embodiments, the method decreases CCX-CKR2 activity, thereby reducing angiogenesis. In some embodiments, the agent is a polynucleotide that inhibits expression of CCX-CKR2. In some embodiments, the agent is an antagonist selected from the group consisting of a polypeptide, an antibody and an agent with a mass of less than 1,500 daltons. In some embodiments, the agent is a polynucleotide that inhibits expression of CCX-CKR2. In some embodiments, the agent is administered in combination with a second anti-angiogenic agent.

In some embodiments, the agent is a CCX-CKR2 antagonist. In some embodiments, the antagonist is selected from a polypeptide, an antibody and an agent with a mass of less than 1,500 daltons.

In some embodiments, the subject has cancer. In some embodiments, the subject has a solid tumor and the agent is targeted or delivered to the tumor.

In some embodiments, an amount of a chemotherapeutic agent or radiation is administered to the subject in combination with the agent. In some embodiments, the amount is sub-therapeutic when the chemotherapeutic agent or radiation is administered alone.

In some embodiments, the subject does not have cancer.

In some embodiments, angiogenesis is reduced in a tissue selected from an eye, skin, joint, ovarian tissue or endometrial tissue. In some embodiments, the agent is used as a birth control agent.

In some embodiments, the subject has arthritis, and the agent is administered in an amount effective to reduce arthritis symptoms in the subject.

The present invention also provides pharmaceutical compositions comprising an amount of a chemotherapeutic agent in combination with an agent that decreases CCX-CKR2 activity. In some embodiments, the amount is sub-therapeutic when the chemotherapeutic agent is administered alone.

The present invention also provides pharmaceutical compositions comprising an agent that increases CCX-CKR2 activity and a second agent that promotes angiogenesis.

The present invention also provides pharmaceutical compositions comprising an agent that decreases CCX-CKR2 activity and a second agent that decreases angiogenesis.

The present invention also provides pharmaceutical compositions comprising an agent that decreases CCX-CKR2 activity and a second anti-arthritis agent.

Definitions

“RDC1,” designated herein as “CCX-CKR2” refers to a seven-transmembrane domain presumed G-protein coupled receptor (GPCR). The CCX-CKR2 dog ortholog was originally identified in 1991. See, Libert et al. Science 244:569-572 (1989). The dog sequence is described in Libert et al., Nuc. Acids Res. 18(7):1917 (1990). The mouse sequence is described in, e.g., Heesen et al., Immunogenetics 47:364-370 (1998). The human sequence is described in, e.g., Sreedharan et al., Proc. Natl. Acad. Sci. USA 88:4986-4990 (1991), which mistakenly described the protein as a receptor of vasoactive intestinal peptide. “CCX-CKR2” includes sequences that are substantially similar to or conservatively modified variants of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10.

A “subject” refers to an animal, including a human, mouse, rat, dog or other mammal.

A “chemotherapeutic agent” refers to an agent, which when administered to an individual is sufficient to cause inhibition, slowing or arresting of the growth of cancerous cells, or is sufficient to produce a cytotoxic effect in cancerous cells. Accordingly, the phrase “chemotherapeutically effective amount” describes an amount of a chemotherapeutic agent administered to an individual, which is sufficient to cause inhibition, slowing or arresting of the growth of cancerous cells, or which is sufficient to produce a cytotoxic effect in cancerous cells. A “sub-therapeutic amount” refers to an amount less than is sufficient to cause inhibition, slowing or arresting of the growth of cancerous cells, or which is less than sufficient to produce a cytotoxic effect in cancerous cells.

“Antibody” refers to a polypeptide substantially encoded by an immunoglobulin gene or immunoglobulin genes, or fragments thereof, which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 10 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term “antibody,” as used herein, also includes antibody fragments either produced by the modification of whole antibodies or those synthesized de novo using recombinant DNA methodologies (e.g., single chain Fv).

“Humanized” antibodies refer to a molecule having an antigen binding site that is substantially derived from an immunoglobulin from a non-human species and the remaining immunoglobulin structure of the molecule based upon the structure and/or sequence of a human immunoglobulin. The antigen binding site may comprise either complete variable domains fused onto constant domains or only the complementarity determining regions (CDRs) grafted onto appropriate framework regions in the variable domains. Antigen binding sites may be wild type or modified by one or more amino acid substitutions, e.g., modified to resemble human immunoglobulin more closely. Some forms of humanized antibodies preserve all CDR sequences (for example, a humanized mouse antibody which contains all six CDRs from the mouse antibodies). Other forms of humanized antibodies have one or more CDRs (one, two, three, four, five, six) which are altered with respect to the original antibody.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants, e.g., proteins at least 80%, 85%, 90%, 95% or 99% identical to SEQ ID NO:2. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

A “ligand” refers to an agent, e.g., a polypeptide or other molecule, capable of binding to a receptor.

As used herein, “an agent that binds to a chemokine receptor” refers to an agent that binds to the chemokine receptor with a high affinity. “High affinity” refers to an affinity sufficient to induce a pharmacologically relevant response, e.g., the ability to significantly compete for binding with a natural chemokine ligand to a chemokine receptor at pharmaceutically relevant concentrations (e.g., at concentrations lower than about 10⁻⁵ M.) Some exemplary agents with high affinity will bind to a chemokine receptor with an affinity greater than 10⁻⁶ M, and sometimes greater than 10⁻⁷ M, 10⁻⁸ M or 10⁻⁹. An agent that fails to compete for binding with a natural receptor ligand when the agent is in a concentrations lower than 10⁻⁴ M will be considered to “not bind” for the purposes of the invention.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same over a specified region, e.g., of the entire CCX-CKR2 polypeptide, when compared and aligned for maximum correspondence over a comparison window, or designated region as measured using one of the following sequence comparison algorithms or by manual alignment and visual inspection. Optionally, the identity exists over a region that is at least about 50 nucleotides or amino acids in length, or more preferably over a region that is 100 to 500 or 1000 or more nucleotides or amino acids in length.

The term “similarity,” or percent “similarity,” in the context of two or more polypeptide or polynucleotide sequences, refer to two or more sequences or subsequences that have a specified percentage of amino acid residues that have a specified percentage of amino acid residues or nucleotides, respectively, the same (i.e., 60%, optionally 65%, 70%, 75%, 80%, 85%, 90%, 95%, or 99%) over a specified region or the entire sequence of the CCX-CKR2 polypeptide or polynucleotide when compared and aligned for maximum correspondence over a comparison window, or designated region as measured using one of the following sequence comparison algorithms or by manual alignment and visual inspection. Optionally, this identity exists over a region that is at least about 50 amino acids or nucleotides in length, or more preferably over a region that is at least about 100 to 500 or 1000 or more amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Modulators” of CCX-CKR2 activity are used to refer to molecules that increase or decrease CCX-CKR2 activity directly or indirectly and includes those molecules identified using in vitro and in vivo assays for CCX-CKR2 binding or signaling. CCX-CKR2 activity can be increased, e.g., by contacting the CCX-CKR2 polypeptide with an agonist, and/or, in some cases, by expressing CCX-CKR2 in a cell. Agonists refer to molecules that increase activity of CCX-CKR2. Agonists are agents that, e.g., bind to, stimulate, increase, open, activate, facilitate, enhance activation, sensitize or up regulate the activity of CCX-CKR2. Modulators may compete for binding to CCX-CKR2 with known CCX-CKR2 ligands such as SDF-1 and I-TAC and small molecules as described herein.

Antagonists refer to molecules that inhibit CCX-CKR2 activity, e.g., by blocking binding of agonists such as I-TAC or SDF-1. Antagonists are agents that, e.g., bind to, partially or totally block stimulation, decrease, prevent, delay activation, inactivate, desensitize, or down regulate the activity of CCX-CKR2. Antagonists include, e.g., antibodies and small organic molecules.

Modulators include agents that, e.g., alter the interaction of CCX-CKR2 with other signal transduction proteins. Modulators include genetically modified versions of naturally-occurring chemokine receptor ligands, e.g., with altered activity, as well as naturally occurring and synthetic ligands, antagonists, agonists, small chemical molecules, siRNAs and the like. Assays for inhibitors and activators include, e.g., applying putative modulator compounds to a cell expressing CCX-CKR2 and then determining the functional effects on CCX-CKR2 signaling or determining the effect on ligand (e.g., SDF-1 or I-TAC) binding to CCX-CKR2. Samples or assays comprising CCX-CKR2 that are treated with a potential activator, inhibitor, or modulator are compared to control samples without the inhibitor, activator, or modulator to examine the extent of inhibition. Control samples (untreated with inhibitors) are assigned a relative chemokine receptor activity value of 100%. Inhibition of CCX-CKR2 is achieved when CCX-CKR2 activity or expression value relative to the control is less than about 95%, optionally about 90%, optionally about 80%, optionally about 50% or about 25-0%. Activation of CCX-CKR2 is achieved when CCX-CKR2 activity or expression value relative to the control is at least about 105%, about 110%, optionally at least about 105%, about 150%, optionally at least about 105%, about 200-500%, or at least about 105%, about 1000-3000% or higher.

“siRNA” refers to small interfering RNAs, that are capable of causing interference with gene expression and can cause post-transcriptional silencing of specific genes in cells, for example, mammalian cells (including human cells) and in the body, for example, mammalian bodies (including humans). The phenomenon of RNA interference is described and discussed in Bass, Nature 411: 428-29 (2001); Elbahir et al., Nature 411: 494-98 (2001); and Fire et al., Nature 391: 806-11(1998); and WO 01/75164, where methods of making interfering RNA also are discussed. siRNAs generally form double stranded RNA sequences, which triggers degradation of homologous transcripts. The double stranded portion of the siRNA may be formed, for example, from two separate complementary RNA sequences or as one RNA sequence which forms a hairpin structure. The siRNAs based upon the sequences and nucleic acids encoding the gene products disclosed herein typically have fewer than 100 base pairs and can be, e.g., about 30 bps or shorter, and can be made by approaches known in the art, including the use of complementary DNA strands or synthetic approaches. The siRNAs are capable of causing interference and can cause post-transcriptional silencing of specific genes in cells, for example, mammalian cells (including human cells) and in the body, for example, mammalian bodies (including humans). Exemplary siRNAs according to the invention could have up to 29 bps, 25 bps, 22 bps, 21 bps, 20 bps, 15 bps, 10 bps, 5 bps or any integer thereabout or therebetween. Tools for designing optimal inhibitory siRNAs include that available from DNAengine Inc. (Seattle, Wash.) and Ambion, Inc. (Austin, Tex.).

One RNAi technique employs genetic constructs within which sense and anti-sense sequences are placed in regions flanking an intron sequence in proper splicing orientation with donor and acceptor splicing sites. Alternatively, spacer sequences of various lengths may be employed to separate self-complementary regions of sequence in the construct. During processing of the gene construct transcript, intron sequences are spliced-out, allowing sense and anti-sense sequences, as well as splice junction sequences, to bind forming double-stranded RNA. Select ribonucleases then bind to and cleave the double-stranded RNA, thereby initiating the cascade of events leading to degradation of specific mRNA gene sequences, and silencing specific genes.

The term “compound” refers to a specific molecule and includes its enantiomers, diastereomers, polymorphs and salts thereof.

As used herein, the term “heteroatom” is meant to include oxygen (O), nitrogen (N), sulfur (S) and silicon (Si).

The term “substituted” refers to a group that is bonded to a parent molecule or group. Thus, a benzene ring having a methyl substituent is a methyl-substituted benzene. Similarly, a benzene ring having 5 hydrogen substituents would be an unsubstituted phenyl group when bonded to a parent molecule.

The term “substituted heteroatom” refers to a group where a heteroatom is substituted. The heteroatom may be substituted with a group or atom, including, but not limited to hydrogen, halogen, alkyl, alkylene, alkenyl, alkynyl, aryl, arylene, cycloalkyl, cycloalkylene, heteroaryl, heteroarylene, heterocyclyl, carbocycle, hydroxy, alkoxy, aryloxy, and sulfonyl. Representative substituted heteroatoms include, by way of example, cyclopropyl aminyl, isopropyl aminyl, benzyl aminyl, and phenoxy.

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having four or fewer carbon atoms.

The term “alkenyl” refers to a monovalent unsaturated hydrocarbon group which may be linear or branched and which has at least one, and typically 1, 2 or 3, carbon-carbon double bonds. Unless otherwise defined, such alkenyl groups typically contain from 2 to 10 carbon atoms. Representative alkenyl groups include, by way of example, ethenyl, n-propenyl, isopropenyl, n-but-2-enyl, n-hex-3-enyl, and the like.

The term “alkynyl” refers to a monovalent unsaturated hydrocarbon group which may be linear or branched and which has at least one, and typically 1, 2 or 3, carbon-carbon triple bonds. Unless otherwise defined, such alkynyl groups typically contain from 2 to 10 carbon atoms. Representative alkynyl groups include, by way of example, ethynyl, n-propynyl, n-but-2-ynyl, n-hex-3-ynyl, and the like.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom. Non-limiting examples of aryl groups include phenyl, naphthyl and biphenyl, while non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, benzopyrazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl or heteroaryl group is attached to an alkyl group (e.g., benzyl, phenethyl, pyridylmethyl and the like).

The term “arylene” refers to a divalent aromatic hydrocarbon having a single ring (i.e., phenylene) or fused rings (i.e., naphthalenediyl). Unless otherwise defined, such arylene groups typically contain from 6 to 10 carbon ring atoms. Representative arylene groups include, by way of example, 1,2-phenylene, 1,3-phenylene, 1,4-phenylene, naphthalene-1,5-diyl, naphthalene-2,7-diyl, and the like.

The term “aralkyl” refers to an aryl substituted alkyl group. Representative aralkyl groups include benzyl.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “cycloalkyl” refers to a monovalent saturated carbocyclic hydrocarbon group having a single ring or fused rings. Unless otherwise defined, such cycloalkyl groups typically contain from 3 to 10 carbon atoms. Representative cycloalkyl groups include, by way of example, cyclopropyl, cyclobutyl, cyclopentyl, cyclohexyl, and the like.

The term “cycloalkylene” refers to a divalent saturated carbocyclic hydrocarbon group having a single ring or fused rings. Unless otherwise defined, such cycloalkylene groups typically contain from 3 to 10 carbon atoms. Representative cycloalkylene groups include, by way of example, cyclopropane-1,2-diyl, cyclobutyl-1,2-diyl, cyclobutyl-1,3-diyl, cyclopentyl-1,2-diyl, cyclopentyl-1,3-diyl, cyclohexyl-1,2-diyl, cyclohexyl-1,3-diyl, cyclohexyl-1,4-diyl, and the like.

The term “heteroaryl” refers to a substituted or unsubstituted monovalent aromatic group having a single ring or fused rings and containing in the ring at least one heteroatom (typically 1 to 3 heteroatoms) selected from nitrogen, oxygen, or sulfur. Unless otherwise defined, such heteroaryl groups typically contain from 5 to 10 total ring atoms. Representative heteroaryl groups include, by way of example, monovalent species of pyrrole, imidazole, thiazole, oxazole, furan, thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like, where the point of attachment is at any available carbon or nitrogen ring atom.

The term “heteroarylene” refers to a divalent aromatic group having a single ring or fused rings and containing at least one heteroatom (typically 1 to 3 heteroatoms) selected from nitrogen, oxygen or sulfur in the ring. Unless otherwise defined, such heteroarylene groups typically contain from 5 to 10 total ring atoms. Representative heteroarylene groups include, by way of example, divalent species of pyrrole, imidazole, thiazole, oxazole, furan thiophene, triazole, pyrazole, isoxazole, isothiazole, pyridine, pyrazine, pyridazine, pyrimidine, triazine, indole, benzofuran, benzothiophene, benzimidazole, benzthiazole, quinoline, isoquinoline, quinazoline, quinoxaline and the like, where the point of attachment is at any available carbon or nitrogen ring atom.

The terms “heterocyclyl” or “heterocyclic group” refer to a substituted or unsubstituted monovalent saturated or unsaturated (non-aromatic) group having a single ring or multiple condensed rings and containing in the ring at least one heteroatom (typically 1 to 3 heteroatoms) selected from nitrogen, oxygen or sulfur. Unless otherwise defined, such heterocyclic groups typically contain from 2 to 9 total ring atoms. Representative heterocyclic groups include, by way of example, monovalent species of pyrrolidine, morpholine, imidazolidine, pyrazolidine, piperidine, 1,4-dioxane, thiomorpholine, piperazine, 3-pyrroline and the like, where the point of attachment is at any available carbon or nitrogen ring atom.

The term “carbocycle” refers to an aromatic or non-aromatic ring in which each atom in the ring is carbon. Representative carbocycles include cyclohexane, cyclohexene, and benzene.

The terms “halo” or “halogen” refers to fluoro-(—F), chloro-(—Cl), bromo-(—Br), and iodo-(—I).

The term “hydroxy” or “hydroxyl” refers to an —OH group.

The term “alkoxy” refers to an —OR group, where R can be a substituted or unsubstituted alkyl, alkylene, cycloalkyl, or cycloalkylene. Suitable substituents include halo, cyano, alkyl, amino, hydroxy, alkoxy, and amido. Representative alkoxy groups include, by way of example, methoxy, ethoxy, isopropyloxy, and trifluoromethoxy.

The term “aryloxy” refers to an —OR group, where R can be a substituted or unsubstituted aryl or heteroaryl group. Representative aryloxy groups include phenoxy.

The term “sulfonyl” refers to a —S(O)₂— or —S(O)₂R group, where R can be alkyl, alkylene, alkenyl, alkynyl, aryl, cycloalkyl, cycloalkylene, heteroaryl, heteroarylene, heterocyclic, or halogen. Representative sulfonyl groups include, by way of example, sulfonate, sulfonamide, sulfonyl halides, and dipropylamide sulfonate.

The term “condensation” refers to a reaction in which two or more molecules are covalently joined. Likewise, condensation products are the products formed by the condensation reaction.

The term “heterocycle” refers to a saturated or unsaturated non-aromatic cyclic group containing at least one sulfur, nitrogen or oxygen heteroatom. Each heterocycle can be attached at any available ring carbon or heteroatom. Each heterocycle may have one or more rings. When multiple rings are present, they can be fused together or linked covalently. Each heterocycle must contain at least one heteroatom (typically 1 to 5 heteroatoms) selected from nitrogen, oxygen or sulfur. Preferably, these groups contain 0-5 nitrogen atoms, 0-2 sulfur atoms and 0-2 oxygen atoms. More preferably, these groups contain 0-3 nitrogen atoms, 0-1 sulfur atoms and 0-1 oxygen atoms. Non-limiting examples of heterocycle groups include pyrrolidine, piperidine, imidazolidine, pyrazolidine, butyrolactam, valerolactam, imidazolidinone, hydantoin, dioxolane, phthalimide, 1,4-dioxane, morpholine, thiomorpholine, thiomorpholine-S,S-dioxide, piperazine, pyran, pyridone, 3-pyrroline, thiopyran, pyrone, tetrahydrofuran, tetrahydrothiophene and the like.

The above terms (e.g., “alkyl,” “aryl” and “heteroaryl”), in some embodiments, will include both substituted and unsubstituted forms of the indicated radical. Preferred substituents for each type of radical are provided below. For brevity, the terms aryl and heteroaryl will refer to substituted or unsubstituted versions as provided below, while the term “alkyl” and related aliphatic radicals is meant to refer to unsubstituted version, unless indicated to be substituted.

Substituents for the alkyl radicals (including those groups often referred to as alkylene, alkenyl, alkynyl and cycloalkyl) can be a variety of groups selected from: -halogen, —OR′, —NR′R″, —SR′, —SiR′R″ R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —CN and —NO₂ in a number ranging from zero to (2 m′+1), where m′ is the total number of carbon atoms in such radical. R′, R″ and R′″ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl.

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR′S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}-U-, wherein T and U are independently —NH—, —O—, —CH₂— or a single bond, and q is an integer of from 0 to 2. Alternatively, two of the substituents on adjacent atoms of the aryl or heteroaryl ring may optionally be replaced with a substituent of the formula -A-(CH₂)ᵣ-B-, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′- and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occuring amines and the like, such as arginine, betaine, caffeine, choline, N,N′-dibenzylethylenediamine, diethylamine, 2-diethylaminoethanol, 2-dimethylamino ethanol, ethanolamine, ethylenedi amine, N-ethylmorpho line, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### BRIEF DESCRIPTION OF THE DRAWINGS

FIG. 1 illustrates joint diameter of mice as a function of time. The mice were treated with a CCX-CKR2 inhibitor or a vehicle only. * indicates P=0.043 (vehicle vs. CCX-CKR2 inhibitor). ** indicates P=0.0002 (vehicle vs. CCX-CKR2 inhibitor). *** indicates P<0.0001 (vehicle vs. CCX-CKR2 inhibitor).

### DETAILED DESCRIPTION OF THE INVENTION

I. Introduction

The present invention is based in part on the surprising discovery that modulating CCX-CKR2 modulates angiogenesis, wound healing and arthritis. In view of this discovery, the present invention provides methods of modulating angiogenesis and/or wound healing and/or arthritis in a subject by modulating CCX-CKR2. As described in more detail herein, there are a number of different ways to modulate CCX-CKR2 activity.

CCX-CKR2 activity can be up-regulated, for example, by contacting CCX-CKR2 with an agonist that stimulates the receptor's activity. In other embodiments, CCX-CKR2 is expressed in a cell of the subject and, optionally contacted with a CCX-CKR2 agonist. Examples of CCX-CKR2 agonists include, e.g., naturally-occurring agonists such as SDF-1 and I-TAC, as well as antibody-based and small molecules that activate CCX-CKR2.

CCX-CKR2 activity can be decreased, for example, by reducing the expression of CCX-CKR2 or by contacting CCX-CKR2 with an antagonist. Antagonists can for example, compete with naturally-occurring agonists (e.g., SDF-1 or I-TAC) or prevent them from binding CCX-CKR2. Antagonists include, but are not limited to, antibodies that bind to CCX-CKR2 (e.g., those that compete with SDF-1 or I-TAC for binding to CCX-CKR2) as well as small organic molecules (e.g., those described herein).

Those of skill in the art will understand that agents that decrease CCX-CKR2 activity can be combined in pharmaceutical compositions with other anti-angiogenesis agents and/or with chemotherapeutic agents or radiation and/or other anti-arthritis agents. In some cases, the amount of chemotherapeutic agent or radiation is an amount which would be sub-therapeutic if provided without combination with an anti-angiogenic agent. Those of skill in the art will appreciate that “combinations” can involve combinations in treatments (i.e., two or more drugs can be administered as a mixture, or at least concurrently or at least introduced into a subject at different times but such that both are in the bloodstream of a subject at the same time).

II. CCX-CKR2 Polypeptides and Polynucleotides

In numerous embodiments of the present invention, nucleic acids encoding CCX-CKR2 polypeptides of interest will be isolated and cloned using recombinant methods. Such embodiments are used, e.g., to isolate CCX-CKR2 polynucleotides (e.g., SEQ ID NO:1, SEQ ID NO:3, SEQ ID NO:5, SEQ ID NO:7, and SEQ ID NO:9)) for protein expression or during the generation of variants, derivatives, expression cassettes, or other sequences derived from a CCX-CKR2 polypeptide (e.g., SEQ ID NO:2, SEQ ID NO:4, SEQ BD NO:6, SEQ ID NO:8, and SEQ ID NO:10)), to monitor CCX-CKR2 gene expression, for the isolation or detection of CCX-CKR2 sequences in different species, for diagnostic purposes in a patient, e.g., to detect mutations in CCX-CKR2 or to detect expression of CCX-CKR2 nucleic acids or CCX-CKR2 polypeptides. In some embodiments, the sequences encoding CCX-CKR2 are operably linked to a heterologous promoter. In some embodiments, the nucleic acids of the invention are from any mammal, including, in particular, e.g., a human, a mouse, a rat, a dog, etc.

In some cases, the CCX-CKR2 polypeptides of the invention comprise the extracellular amino acids of the human CCX-CKR2 sequence (e.g., of SEQ ID NO:2, SEQ ID NO:4, SEQ ID NO:6, SEQ ID NO:8, and SEQ ID NO:10)) while other residues are either altered or absent. In other embodiments, the CCX-CKR2 polypeptides comprise ligand-binding fragments of CCX-CKR2. For example, in some cases, the fragments bind I-TAC and/or SDF1. The structure of seven trans-membrane receptors (of which CCX-CKR2 is one) are well known to those skilled in the art and therefore trans-membrane domains can be readily determined. For example, readily available hydrophobicity algorithms can be found on the internet at the G Protein-Coupled Receptor Data Base (GPCRDB), e.g., http://www.gpcr.org/7tm/seq/DR/RDC1_HUMAN.TABDR.html or http://www.gpcr.org/7tm/seq/vis/swac/P25106.html.

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

Appropriate primers and probes for identifying the genes encoding CCX-CKR2 from mammalian tissues can be derived from the sequences provided herein (e.g., SEQ ID NO:1). For a general overview of PCR, see, Innis et al. PCR Protocols: A Guide to Methods and Applications, Academic Press, San Diego (1990).

III. Modulators of CCX-CKR2

A. Methods of Identifying Modulators of Chemokine Receptors

A number of different screening protocols can be utilized to identify agents that modulate the level of activity or function of CCX-CKR2 in cells, particularly in mammalian cells, and especially in human cells. In general terms, the screening methods involve screening a plurality of agents to identify an agent that interacts with CCX-CKR2 (or an extracellular domain thereof), for example, by binding to CCX-CKR2, preventing a ligand (e.g., I-TAC and/or SDF1) from binding to CCX-CKR2 or activating CCX-CKR2. In some embodiments, an agent binds CCX-CKR2 with at least about 1.5, 2, 3, 4, 5, 10, 20, 50, 100, 300, 500, or 1000 times the affinity of the agent for another protein.

1. Chemokine Receptor Binding Assays

In some embodiments, CCX-CKR2 modulators are identified by screening for molecules that compete with a ligand of CCX-CKR2 such as SDF1 or I-TAC. Those of skill in the art will recognize that there are a number of ways to perform competition analyses. In some embodiments, samples with CCX-CKR2 are pre-incubated with a labeled CCX-CKR2 ligand and then contacted with a potential competitor molecule. Alteration (e.g., a decrease) of the quantity of ligand bound to CCX-CKR2 indicates that the molecule is a potential CCX-CKR2 modulator.

Preliminary screens can be conducted by screening for agents capable of binding to a CCX-CKR2, as at least some of the agents so identified are likely chemokine receptor modulators. The binding assays usually involve contacting CCX-CKR2 with one or more test agents and allowing sufficient time for the protein and test agents to form a binding complex. Any binding complexes formed can be detected using any of a number of established analytical techniques. Protein binding assays include, but are not limited to, immunohistochemical binding assays, flow cytometry, radioligand binding, europium labeled ligand binding, biotin labeled ligand binding or other assays which maintain the conformation of CCX-CKR2. The chemokine receptor utilized in such assays can be naturally expressed, cloned or synthesized. Binding assays may be used to identify agonists or antagonists. For example, by contacting CCX-CKR2 with a potential agonist and measuring for CCX-CKR2 activity, it is possible to identify those molecules that stimulate CCX-CKR2 activity.

2. Cells and Reagents

The screening methods of the invention can be performed as in vitro or cell-based assays. In vitro assays are performed for example, using membrane fractions or whole cells comprising CCX-CKR2. Cell based assays can be performed in any cells in which CCX-CKR2 is expressed.

Cell-based assays involve whole cells or cell fractions containing CCX-CKR2 to screen for agent binding or modulation of activity of CCX-CKR2 by the agent. Exemplary cell types that can be used according to the methods of the invention include, e.g., any mammalian cells including leukocytes such as neutrophils, monocytes, macrophages, eosinophils, basophils, mast cells, and lymphocytes, such as T cells and B cells, leukemias, Burkitt's lymphomas, tumor cells, endothelial cells, pericytes, fibroblasts, cardiac cells, muscle cells, breast tumor cells, ovarian cancer carcinomas, cervical carcinomas, glioblastomas, liver cells, kidney cells, and neuronal cells, as well as fungal cells, including yeast. Cells can be primary cells or tumor cells or other types of immortal cell lines. Of course, CCX-CKR2 can be expressed in cells that do not express an endogenous version of CCX-CKR2.

In some cases, fragments of CCX-CKR2, as well as protein fusions, can be used for screening. When molecules that compete for binding with CCX-CKR2 ligands are desired, the CCX-CKR2 fragments used are fragments capable of binding the ligands (e.g., capable of binding I-TAC or SDF1). Alternatively, any fragment of CCX-CKR2 can be used as a target to identify molecules that bind CCX-CKR2. CCX-CKR2 fragments can include any fragment of, e.g., at least 20, 30, 40, 50 amino acids up to a protein containing all but one amino acid of CCX-CKR2. Typically, ligand-binding fragments will comprise transmembrane regions and/or most or all of the extracellular domains of CCX-CKR2.

3. Signaling or Adhesion Activity

In some embodiments, signaling triggered by CCX-CKR2 activation is used to identify CCX-CKR2 modulators. Signaling activity of chemokine receptors can be determined in many ways. For example, signaling can be determined by detecting chemokine receptor-mediated cell adhesion. Interactions between chemokines and chemokine receptors can lead to rapid adhesion through the modification of integrin affinity and avidity. See, e.g., Laudanna, Immunological Reviews 186:37-46 (2002).

Signaling can also be measured by determining, qualitatively and quantitatively, secondary messengers, such as cyclic AMP or inositol phosphates, as well as phosphorylation or dephosphorylation events can also be monitored. See, e.g., Premack, et al. Nature Medicine 2: 1174-1178 (1996) and Bokoch, Blood 86:1649-1660 (1995).

In addition, other events downstream of CCX-CKR2 activation can also be monitored to determine signaling activity. Downstream events include those activities or manifestations that occur as a result of stimulation of a chemokine receptor. Exemplary downstream events include, e.g., changed state of a cell (e.g., from normal to cancer cell or from cancer cell to non-cancerous cell). Cell responses include adhesion of cells (e.g., to endothelial cells). Established signaling cascades involved in angiogenesis (e.g., VEGF-mediated signaling) can also be monitored for effects caused by CCX-CKR2 modulators. The ability of agents to promote angiogenesis can be evaluated, for example, in chick chorioallantoic membrane, as discussed by Leung et al. (1989) Science 246:1306-1309. Another option is to conduct assays with rat comeas, as discussed by Rastinejad et al. (1989) Cell 56:345-355. Other assays are disclosed in U.S. Pat. No. 5,840,693. Ovarian angiogenesis models can also be used (see, e.g., Zimmerman, R. C., et al. (2003) J. Clin. Invest. 112:659-669; Zimmerman, R. C., et al. (2001) Microvasc. Res. 62:15-25; and Hixenbaugh, E. A., et al. (1993) Anat. Rec. 235: 487-500).

As described in greater detail in the examples, expression of CCX-CKR2 results in extended cell survival of CCX-CKR2-expressing cells grown in low serum conditions as compared to cells not expressing CCX-CKR2 grown under the same conditions. Thus, antagonism of CCX-CKR2 is expected to reduce cell survival, whereas activation (e.g., via agonists) is expected to increase cell survival. Consequently, cell survival and apoptosis can serve as a readout for CCX-CKR2 activity.

A wide variety of cell death and apoptosis assays can be incorporated into screening methods to identify modulators of CCX-CKR2. In general, assays of this type typically involve subjecting a population of cells to conditions that induce cell death or apoptosis, usually both the in the presence and absence of a test compound that is a potential modulator of cell death or apoptosis. An assay is then conducted with the cells, or an extract thereof, to assess what effect the test agent has on cell death or apoptosis by comparing the extent of cell death or apoptosis in the presence and absence of the test agent. Instead of assaying for cell death or apoptosis, the opposite type of assay can be performed, namely assaying for cell survival, as well as related activities such as cell growth and cell proliferation. Regardless of the particular type of assay, some assays are conducted in the presence of a ligand that activates CCX-CKR2 such as I-TAC or SDF-1.

A variety of different parameters that are characteristic of cell death and apoptosis can be assayed for in the present screening methods. Examples of such parameters include, but are not limited to, monitoring activation of cellular pathways for toxicological responses by gene or protein expression analysis, DNA fragmentation, changes in the composition of cellular membranes, membrane permeability, activation of components of death-receptors or downstream signaling pathways (e.g., caspases), generic stress responses, NF-kappa B activation and responses to mitogens.

In view of the role that CCX-CKR2 plays in reducing apoptosis, another approach is to assay for the opposite of apoptosis and cell death, namely to conduct screens in which cell survival or cell proliferation is detected. Cell survival can be detected, for instance, by monitoring the length of time that cells remain viable, the length of time that a certain percentage of the original cells remain alive, or an increase in the number of cells. These parameters can be monitored visually using established techniques.

Another assay to assess apoptosis involves labeling cells with Annexin V (conjugated to Alexa Fluor(r) 488 dye) and Propidium Iodide (PI) (Molecular Probes, Eugene Oreg.). PI, a red fluorescent nucleic acid-binding dye, is impermeant to both live and apoptotic cells. PI only labels necrotic cells by tightly biding to the nucleic acids in the cell. Annexin V takes advantage of the fact that apoptotic cells translocate phosphatidylserine (PS) to the external surface of the cell. Annexin V is a human anti-coagulant with high affinity for (PS). Apoptotic cells, but not live cells, express PS on their outer surface. Annexin V (labeled with Alexa Fluor(r) 488 dye) labels these cells with green fluorescence. Cells can then be analyzed on a fluorescence activated cell sorter (FACS) to assess the fluorescence in the red and green channels: apoptotic cells (Annexin positive, PI negative) fluoresce only in the green channel; live cells (Annexin negative, PI negative) exhibit low fluorescence in both the red and green channels; and necrotic or dead cells (Annexin positive, PI positive) are strongly positive in both the red and green channels.

Other screening methods are based on the observation that expression of certain regulatory proteins is induced by the presence or activation of CCX-CKR2. Detection of such proteins can thus be used to indirectly determine the activity of CCX-CKR2. As described in greater detail in the examples below, a series of ELISA investigations were conducted to compare the relative concentration of various secreted proteins in the cell culture media for cells transfected with CCX-CKR2 and untransfected cells. Through these studies it was determined that CCX-CKR2 induces the production of a number of diverse regulatory proteins, including growth factors, chemokines, metalloproteinases and inhibitors of metalloproteinases. Thus, some of the screening methods that are provided involve determining whether a test agent modulates the production of certain growth factors, chemokines, metalloproteinases and inhibitors of metalloproteinases by CCX-CKR2. In some instances, the assays are conducted with cells (or extracts thereof) that have been grown under limiting serum conditions as this was found to increase the production of the CCX-CKR2-induced proteins (see examples).

The following proteins are examples of the various classes of proteins that were detected, as well as specific proteins within each class: (1) growth factors (e.g., GM-CSF); (2) chemokines (e.g., RANTES, MCP-1); (3) metalloproteinase (e.g., MMP3); and (4) inhibitor of metalloproteinase (e.g., TIMP-1). It is expected that other proteins in these various classes can also be detected.

These particular proteins can be detected using standard immunological detection methods that are known in the art. One approach that is suitable for use in a high-throughput format, for example, are ELISAs that are conducted in multi-well plates. An ELISA kit for detecting TIMP-1 is available from DakoCytomation (Product Code No. EL513). Further examples of suppliers of antibodies that specifically bind the proteins listed above are provided in the examples below. Proteins such as the metalloproteinases that are enzymes can also be detected by known enzymatic assays.

In other embodiments, potential modulators of CCX-CK2 are tested for their ability to modulate cell adhesion. Tumor cell adhesion to endothelial cell monolayers has been studied as a model of metastatic invasion (see, e.g., Blood and Zetter, Biovhem. Biophys. Acta, 1032, 89-119 (1990). These monolayers of endothelial cells mimic the lymphatic vasculature and can be stimulated with various cytokines and growth factors (e.g., TNFalpha and IL-1beta). Cells expressing CCX-CKR2 can be evaluated for the ability to adhere to this monolayer in both static adhesion assays as well as assays where cells are under flow conditions to mimic the force of the vasculature in vivo. Additionally, assays to evaluate adhesion can also be performed in vivo (see, e.g., von Andrian, U. H. Microcirculation. 3(3):287-300 (1996)).

4. Validation

Agents that are initially identified by any of the foregoing screening methods can be further tested to validate the apparent activity. Preferably such studies are conducted with suitable animal models. The basic format of such methods involves administering a lead compound identified during an initial screen to an animal that serves as a disease model for humans and then determining if the disease (e.g., cancer, myocardial infarction, wound healing, or other diseases related to angiogenesis) is in fact modulated and/or the disease or condition is ameliorated. The animal models utilized in validation studies generally are mammals of any kind. Specific examples of suitable animals include, but are not limited to, primates, mice, rats and zebrafish.

In some embodiments, arthritis animal models are used to screen and/or validate therapeutic uses for agents that modulate CCX-CKR2. Exemplary arthritis animal models include, e.g., the collagen-induced arthritis (CIA) animal model.

B. Agents that Interact with CCX-CKR2

Modulators of CCX-CKR2 (e.g., antagonists or agonists) can include, e.g., antibodies (including monoclonal, humanized or other types of binding proteins that are known in the art), small organic molecules, siRNAs, CCX-CKR2 polypeptides or variants thereof, chemokines (including but not limited to SDF-1 and/or I-TAC), chemokine mimetics, chemokine polypeptides, etc.

The agents tested as modulators of CCX-CKR2 can be any small chemical compound, or a biological entity, such as a polypeptide, sugar, nucleic acid or lipid. Alternatively, modulators can be genetically altered versions, or peptidomimetic versions, of a chemokine or other ligand. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, the agents have a molecular weight of less than 1,500 daltons, and in some cases less than 1,000, 800, 600, 500, or 400 daltons. The relatively small size of the agents can be desirable because smaller molecules have a higher likelihood of having physiochemical properties compatible with good pharmacokinetic characteristics, including oral absorption than agents with higher molecular weight. For example, agents less likely to be successful as drugs based on permeability and solubility were described by Lipinski et al. as follows: having more than 5H-bond donors (expressed as the sum of OHs and NHs); having a molecular weight over 500; having a LogP over 5 (or MLogP over 4.15); and/or having more than 10H-bond acceptors (expressed as the sum of Ns and Os). See, e.g., Lipinski et al. Adv Drug Delivery Res 23:3-25 (1997). Compound classes that are substrates for biological transporters are typically exceptions to the rule.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks.” For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

C. Inhibitors of Angiogenesis

Inhibitors of CCX-CKR2 can include, e.g., antibody antagonists, peptide antagonists, siRNA molecules or small molecules antagonists.

Generation of antibodies is well known in the art. In some embodiments, antibodies specific for CCX-CKR2 are screened for their ability to compete with CCX-CKR2 agonists such as I-TAC or SDF-1. Antibodies include any type of immunological affinity agent including antibody variants or fragments, single chain antibodies, humanized or human antibodies, etc.

In other embodiments, peptide antagonists are provided. Peptide antagonists can be readily selected using any number of well known display technologies to identify peptides that interact with CCX-CKR2.

In other embodiments, siRNA molecules are used to inhibit expression of CCX-CKR2. See, e.g., U.S. Patent Publication No. 2004/0019001 for a description of various compositions of siRNA molecules as well as how to identify siRNA sequences. For example, the target sequence is parsed in silico into a list of all fragments or subsequences of a particular length, for example 23 nucleotide fragments, contained within the target sequence. Following analysis of their structure for desirable features, the siRNA molecules are screened in an in vitro, cell culture or animal model system to identify the most active siRNA molecule or the most preferred target site within the target RNA sequence.

In some embodiments, the modulators of CCX-CKR2 are small organic molecules. In one embodiment, the active compounds (i.e., CCX-CKR2 modulators) of the present invention have the general structure (I): 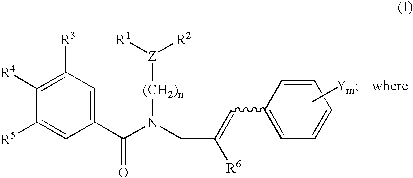
m is an integer from 1 to 5 and each Y that substitutes the benzyl ring is independently selected from the group consisting of hydrogen, alkyl, halo substituted alkyl, alkylene, alkenyl, alkynyl, cycloalkyl, cycloalkylene, halogen, heterocyclic, aryl, arylene, heteroaryl, heteroarylene, hydroxy, alkoxy, and aryloxy,
n is 0, 1, 2 or 3;
Z is —CH— or —N—;
R¹ and R² are each independently alkyl or hydrogen, or Z in combination with R¹ and R² form a 5- or 6-membered ring comprising at least one nitrogen and optionally comprising one or more additional heteroatoms, where
   said 5-6-membered ring is optionally and independently substituted with one or more moieties selected from the group consisting of alkyl, alkenyl, phenyl, benzyl, sulfonyl, and substituted heteroatom;
R³, R⁴, and R⁵ are each independently selected from the group consisting of hydrogen, alkyl, halo substituted alkyl, alkylene, alkenyl, alkynyl, cycloalkyl, cycloalkylene, heterocyclic, aryl, arylene, heteroaryl, heteroarylene, hydroxy, alkoxy, and aryloxy; and
R⁶ is alkyl or hydrogen;
   provided that if Z is nitrogen and R¹ and R² together with Z form a morpholinyl group, then n is 3, and at least one of R³, R⁴, and R⁵ is hydroxy, alkoxy, or aryloxy; or
   provided that if n=1, Z is carbon and R¹ and R² is combination is not —CH₂CH₂NCH₂CH₂—; or
   provided that if R¹ together with R² is —CH(CH₃)(CH₂)₄—, then Z is —CH—; or
   provided that if R⁵ is t-butyl, then R³ is hydrogen; or
   provided that if R⁴ and R⁵ together form a 5-membered ring, then at least one of the atoms bonded to the phenyl ring is carbon. See, U.S. Provisional Patent Application No. 60/434,912, filed Dec. 20, 2002 and U.S. Provisional Patent Application No. 60/516,151, filed Dec. 20, 2003.

The wavy bond connecting the olefin to the substituted phenyl ring signifies that the ring may be either cis or trans to R⁶. In a preferred embodiment, n is 1, 2, or 3. In another preferred embodiment, n is 2 or 3. In a further preferred embodiment, n is 3.

In another embodiment, preferred compounds have the general structure (I), where R⁶ is hydrogen. In a further embodiment, preferred compounds have the general structure (I), where R⁶ is methyl.

In another embodiment, preferred compounds have the general structure (I), where R³, R⁴, and R⁵ are independently hydrogen, hydroxy, alkyl, alkoxy, aryloxy, and halo substituted alkyl. More preferably, R³, R⁴, and R⁵ are independently alkoxy or hydrogen. In another embodiment, preferred compounds have the general structure (I), where R⁴ is hydrogen and R³ and R⁵ are alkoxy (—OR), including trifluoroalkoxy groups such as trifluoromethoxy and (—OCH₂CF₃). In a further embodiment, R³ is hydrogen and R⁴ and R⁵ are alkoxy. In either of these embodiments, the alkoxy group may be methoxy (—OCH₃) or ethoxy (—OCH₂CH₃).

In another embodiment, preferred compounds have the general structure (I), where R⁴ and R⁵ together form a heterocyclic, aryl, or heteroaryl ring. In another preferred embodiment, R³ is hydrogen and R⁴ and R⁵ together are —O(CH₂)₃O—, —(CH)₄—, or —N(CH)₂N—.

In another embodiment, preferred compounds have the general structure (I), where Z is nitrogen and Z in combination with R¹ and R² form a heteroaryl or heterocyclic group. In a preferred embodiment, compounds have the general structure (I), where Z is CH and Z in combination with R¹ and R² form a heteroaryl or heterocyclic group. More preferable compounds have the general structure (I), where Z is CH and Z in combination with R¹ and R² form a heterocyclic group containing nitrogen. In a further embodiment, Z in combination with R¹ and R² form a substituted or unsubstituted morpholinyl, pyrrolidinyl, piperidinyl, or piperazinyl group.

Preferred substituents for the heteroaryl or heterocyclic group include alkyl, alkenyl, alkynyl, cycloalkyl, aryl, aralkyl, heteroaryl, alkoxy, hydroxy, heteroatoms, and halides. In an especially preferred embodiment, the heteroaryl or heterocyclic group is substituted with benzyl, phenyl, methyl, ethyl, cyclohexyl, methoxy-methyl (—CH₂OCH₃), or cyclohexyl-methyl (—CH₂(C₆H₁₁)) groups.

In one embodiment, a preferred compound has the general structure (I), where Z in combination with R¹ and R² is an alkyl- or methoxy-methyl-substituted pyrrolidinyl group; a benzyl-, phenyl-, methyl-, ethyl-, or substituted heteroatom substituted piperidinyl group; or a benzyl-, phenyl-, or sulfonyl-substituted piperazinyl group. Especially preferred substituted heteroatom groups include alkoxy, aminyl, cycloalkyl aminyl, alkyl aminyl, cyclopropyl aminyl, isopropyl aminyl, benzyl aminyl, and phenoxy. Preferably, the substituted heteroatom is at the 3 position of the piperidinyl ring.

In another aspect, preferred compounds have the general structure (I), where Z in combination with R¹ and R² is

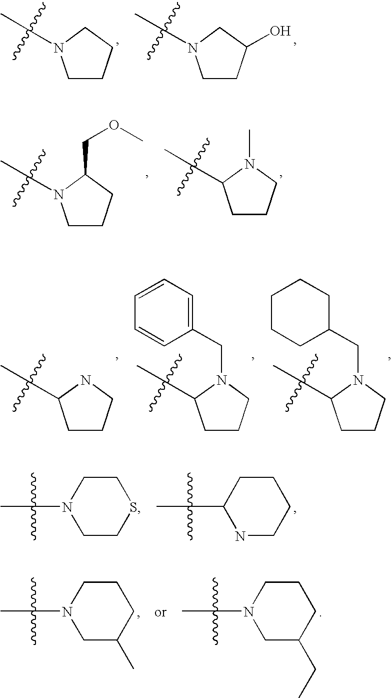

Preferred compounds having the general structure (I) can also have Z as a nitrogen atom, have R¹ and R² each as alkyl or methyl groups, or have R¹ and R² together forming —C(C(O)N(CH₃)₂)(CH₂)₃—.

In another embodiment, Z in combination with R¹ and R² form a 5-membered ring including nitrogen and optionally including one or more additional heteroatoms. In this embodiment, n is preferably 1 and Z is preferably —CH—. In an especially preferred embodiment of this type, Z in combination with R¹ and R² is 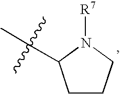
where R⁷ is preferably hydrogen, alkyl, aryl, or aralkyl.

In another preferred embodiment R⁷ can be a halogenated benzyl or phenyl group. In a further embodiment, R⁷ is preferably hydrogen, methyl, ethyl, benzyl, or para-fluoro-phenyl.

In another embodiment, the active compounds of the present invention have the general structure (II): 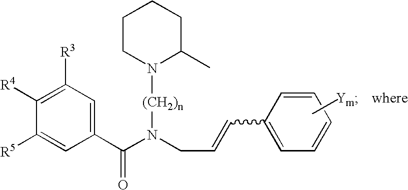
m is an integer from 1 to 5;
each Y that substitutes the benzyl ring is independently selected from the group consisting of hydrogen, alkyl, halo substituted alkyl, alkylene, alkenyl, alkynyl, cycloalkyl, cycloalkylene, halogen, heterocyclic, aryl, arylene, heteroaryl, heteroarylene, hydroxy, and alkoxy,
n is 1, 2 or 3; and
R³, R⁴, and R⁵ are each independently selected from the group consisting of hydrogen, alkyl, halo substituted alkyl, alkylene, alkenyl, alkynyl, cycloalkyl, cycloalkylene, heterocyclic, aryl, arylene, heteroaryl, heteroarylene, hydroxy, alkoxy, and aryloxy.

As in structure (I) above, the wavy bond connecting the olefin to the substituted phenyl ring signifies that the ring may be either cis or trans.

In another embodiment, preferred compounds may have the general structure (II), where n is 3. In another embodiment, preferred compounds may have the general structure (II), where R³, R⁴, and R⁵ are substituted as described for structure (I) above. At present, especially preferred compounds have the general structure (II), where R³, R⁴, and R⁵ are alkoxy or methoxy.

While many synthetic routes known to those of ordinary skill in the art may be used to synthesize the active compounds of the present invention, a general synthesis method is given below in Scheme I.

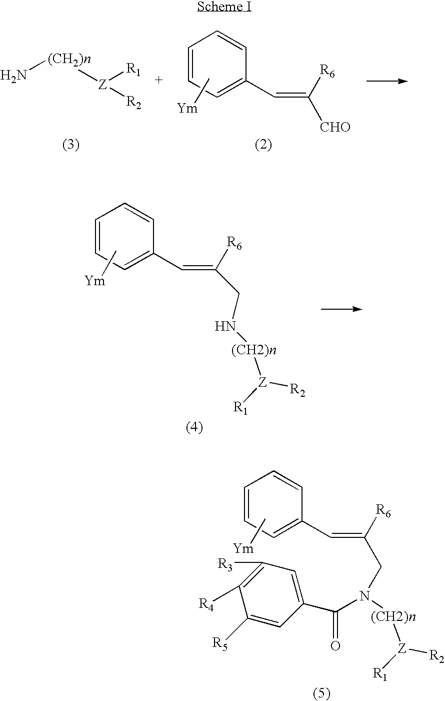

In Scheme I, aldehyde (2) undergoes a condensation reaction with primary amine (3) via reductive amination. Suitable primary amines are commercially available from Aldrich, Milwaukee, Wis., for example, or may be synthesized by chemical routes known to those of ordinary skill in the art.

The amination reaction may be carried out with a reducing agent in any suitable solvent, including, but not limited to tetrahydrofuran (THF), dichloromethane, or methanol to form the intermediate (4). Suitable reducing agents for the condensation reaction include, but are not limited to, sodium cyanoborohydride (as described in Mattson, et al., J. Org. Chem. 1990, 55, 2552 and Barney, et al., Tetrahedron Lett. 1990, 31, 5547); sodium triacethoxyborohydride (as described in Abdel-Magid, et al., Tetrahedron Lett. 31:5595 (1990)); sodium borohydride (as described in Gribble; Nutaitis Synthesis. 709 (1987)); iron pentacarbonyl and alcoholic KOH (as described in Watabane, et al., Tetrahedron Lett. 1879 (1974)); and BH₃-pyridine (as described in Pelter, et al., J. Chem. Soc., Perkin Trans. 1:717 (1984)).

The transformation of intermediate (4) to compound (5) may be carried out in any suitable solvent, such as tetrahydrofuran or dichloromethane, with a suitably substituted acyl chloride in presence of a base. Tertiary amine bases are preferred. Especially preferred bases include triethylamine and Hunnings base.

Alternatively, the transformation of intermediate (4) to compound (5) can also be obtained with a suitable coupling reagent, such as 1-ethyl-3-(3-dimethylbutylpropyl) carbodiimide or Dicyclohexyl-carbodiimide (as described in B. Neises and W. Steglich, Angew. Chem., Int. Ed. Engl. 17:522 (1978)), in the presence of a catalyst, such as 4-N,N-dimethylamino-pyridine, or in the presence of hydroxybenzotriazole (as described in K. Horiki, Synth. Commun. 7:251).

In one aspect, the modulators of CCX-CKR2 are compounds having the formula: 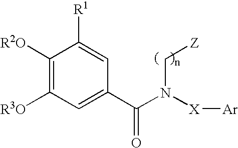
and all pharmaceutically acceptable salts thereof, wherein the subscript n is an integer of from 1 to 3; the symbol R¹ represents a hydrogen, halogen, C₁₋₈ alkoxy, C₁₋₈ alkyl, C₁₋₈ haloalkyl, C₃₋₆ cycloalkyl, C₃₋₆ cycloalkoxy, C₃₋₆ cycloalkyl C₁₋₄ alkyl or C₃₋₆ cycloalkyl C₁₋₄ alkoxy; the symbols R² and R³ are each members independently selected from C₁₋₈ alkyl and C₁₋₈ haloalkyl, or are optionally combined with the oxygen atoms to which each is attached to from a five- to ten-membered ring; the letter X represents a bond or CH₂; the symbol Ar represents a linked- or fused-bicyclic aromatic ring system; and the letter Z represents a five-, six- or seven-membered saturated nitrogen heterocyclic ring that is optionally substituted with from one to four R⁴ substituents independently selected from C₁₋₈ alkyl, C₁₋₈ haloalkyl, C₃₋₆ cycloalkyl, C₂₋₈ alkenyl, C₂₋₈ alkynyl, —COR^{a}, —CO₂R^{a}, —CONR^{a}R^{b}, —NR^{a}COR^{b}, SO₂R^{a}, —X¹COR^{a}, —X¹CO₂R^{a}, X¹CONR^{a}R^{b}, —X¹NR^{a}COR^{b}, —X SO₂R^{a}, —X¹SO₂NR^{a}R^{b}, —X¹NR^{a}R^{b}, —X¹OR^{a} and —X¹R^{a}, wherein X¹ is selected from C₁₋₄ alkylene and C₂₋₄ alkenylene and each Ra and Rb is independently selected from hydrogen, C₁₋₈ alkyl, C₁₋₈ haloalkyl, C₃₋₆ cycloalkyl and aryl-C₁₋₄alkyl, and wherein the aliphatic portions of each of the R⁴ substituents is optionally substituted with from one to three members selected from —OH, —OR^{m}, —OC(O)NHR^{m}, —OC(O)N(R^{m})₂, —SH, —SR^{m}, —S(O)R^{m}, —S(O)₂R^{m}, —SO₂NH₂, —S(O)₂NHR^{m}, —S(O)₂N(R^{m})₂, —NHS(O)₂R^{m}, —NR^{m}S(O)₂R^{m}, —C(O)NH₂, —C(O)NHR^{m}, —C(O)N(R^{m})₂, —C(O)R^{m}, —NHC(O)R^{m}, —NR^{m}C(O)R^{m}, —NHC(O)NH₂, —NR^{m}C(O)NH₂, —NR^{m}C(O)NHR^{m}, —NHC(O)NHR^{m}, —NR^{m}C(O)N(R^{m})₂, —NHC(O)N(R^{m})₂, —CO₂H, —CO₂R^{m}, —NHCO₂R^{m}, —NR^{m}CO₂R^{m}, —CN, —NO₂, —NH₂, —NHR^{m}, —N(R^{m})₂, —NR^{m}S(O)NH₂ and —NR^{m}S(O)₂NHR^{m}, wherein each R^{m} is independently an unsubstituted C₁₋₆ alkyl.

In some embodiments, Z is selected from 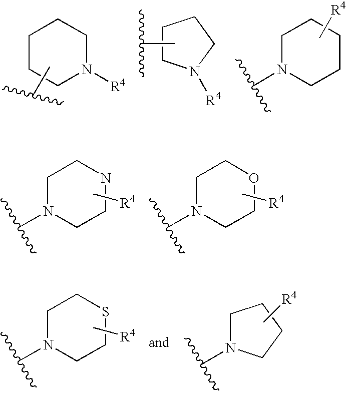
wherein the wavy line indicates the point of attachment to the remainder of the molecule.

In one group of embodiments, Ar is a fused bicyclic aromatic rings system selected from naphthalene, quinoline, benzothiophene, isoquinoline, benzofuran, indole, benzothiazole, benzimidazole, 1,4-benzodioxan, quinoxaline and naphthyridine.

In another group of embodiments, Ar is a linked-bicyclic aromatic ring system selected from biphenyl (wherein the phenyl rings are connected in an ortho-meta- or para-orientation relative to the attachment to the remainder of the compound), 5-phenylthiazolyl, and phenyl substituted with a 5- or 6-membered heteroaryl moiety (e.g., thiazolyl, thienyl, imidazolyl, pyrazolyl, furyl, oxazolyl, pyridyl, pyrimidinyl, pyrazinyl, and the like), wherein each of the above is optionally substituted with from one to six substituents selected from those provided in general for aryl groups (see above).

In some embodiments, the subscript n is 1 or 2. In other embodiments, the symbol R¹ represents a hydrogen or C₁₋₈ alkoxy. In still other embodiments, the symbols R² and R³ each independently represent methyl, ethyl, propyl, isopropyl, butyl, sec-butyl, isobutyl, tert-butyl, or their C₁₋₄ haloalkyl counterparts (e.g., trifluoromethyl, 2,2,2-trichloroethyl, 3-bromopropyl, and the like).

In some embodiments, n is 1 or 2; R¹ is selected from the group consisting of hydrogen and C₁₋₈ alkoxy; R² and R³ are each independently selected from the group consisting of methyl, ethyl, propyl, isopropyl, butyl, sec-butyl, isobutyl, tert-butyl and C₁₋₄ haloalkyl; X is CH₂; Ar is a Ar is a fused bicyclic aromatic ring system selected from the group consisting of naphthalene, quinoline, benzothiophene, isoquinoline, benzofuran, indole, benzothiazole, benzimidazole, 1,4-benzodioxan, quinoxaline and naphthyridine; Z is a member selected from the group consisting of 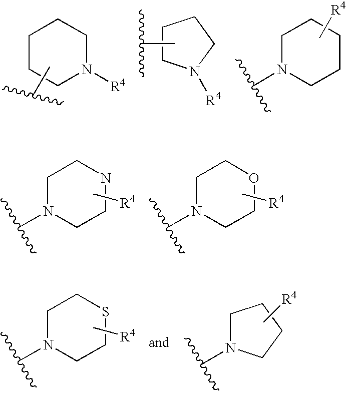
wherein the wavy line indicates the point of attachment to the remainder of the compound; and R⁴ is a member selected from the group consisting of C₁₋₈ alkyl, C₃₋₆ cycloalkyl, —X¹OR^{a} and —X¹R^{a}, wherein X¹ is a member selected from the group consisting of C₁₋₄ alkylene and C₂₋₄ alkenylene and R^{a} is selected from the group consisting of C₁₋₈ alkyl and C₃₋₆ cycloalkyl.

In some embodiments, n is 1 or 2; R¹ is selected from the group consisting of hydrogen and C₁₋₈ alkoxy; R² and R³ are each independently selected from the group consisting of methyl, ethyl, propyl, isopropyl, butyl, sec-butyl, isobutyl, tert-butyl and C₁₋₄ haloalkyl; X is a bond; Ar is a substituted or unsubstituted linked-bicyclic aromatic ring system selected from the group consisting of biphenyl, 5-phenylthiazolyl and phenyl substituted with a 5- or 6-membered heteroaryl moiety; Z is a member selected from the group consisting of 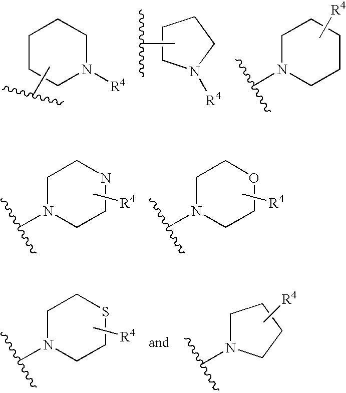
wherein the wavy line indicates the point of attachment to the remainder of the compound; and R⁴ is a member selected from the group consisting of C₁₋₈ alkyl, C₃₋₆ cycloalkyl, —X¹OR^{a} and —X¹R^{a}, wherein X¹ is a member selected from the group consisting of C₁₋₄ alkylene and C₂₋₄ alkenylene and R^{a} is selected from the group consisting of C₁₋₈ alkyl and C₃₋₆ cycloalkyl.

To demonstrate that the compounds described above are useful antagonists for SDF-1 and I-TAC chemokines, the compounds were screened in vitro to determine their ability to displace SDF-1 and I-TAC from the CCX-CKR2 receptor at multiple concentrations. The compounds were combined with mammary gland cells expressing CCX-CKR2 receptor sites in the presence of the ¹²⁵I-labeled SDF-1 and/or 1251I I-TAC chemokine. The ability of the compounds to displace the labeled SDF-1 or I-TAC from the CCX-CKR2 receptor cites at multiple concentrations was then determined with the screening process.

Compounds that were deemed effective SDF-1 and I-TAC antagonists were able to displace at least 50% of the SDF-1 and/or I-TAC chemokine from the CCX-CKR2 receptor at concentrations at or below 1.1 micromolar (μM) and more preferably at concentrations at or below 300 nanomolar (nM). In some cases, it is desirable that compounds can displace at least 50% of the SDF-1 and/or I-TAC from the CCX-CKR2 receptor at concentrations at or below 200 nM. Exemplary compounds that met these criteria are reproduced in Tables I and II below. See also, U.S. Patent Publication No. 2004/0171655 and U.S. Provisional Patent Application No. 60/614,563, filed Sep. 29, 2004.

TABLE I

| o. | Compound |
|---|---|
| 1 | |
| 1 | 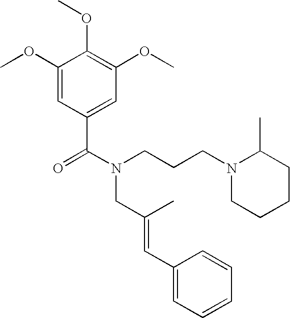 |
| 2 | 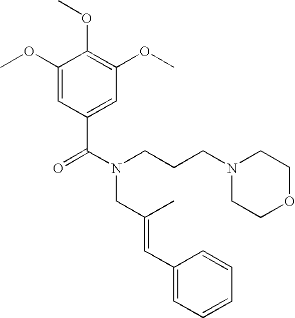 |
| 3 | 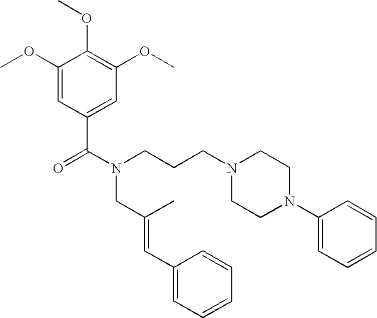 |
| 4 | 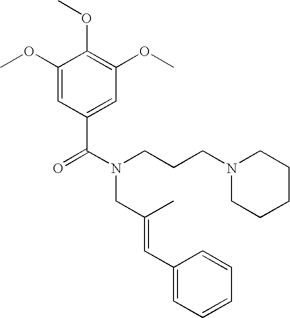 |
| 5 | 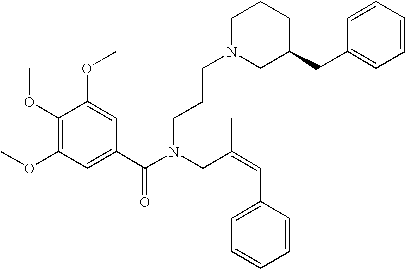 |
| 6 | 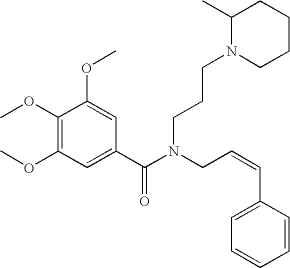 |
| 7 | 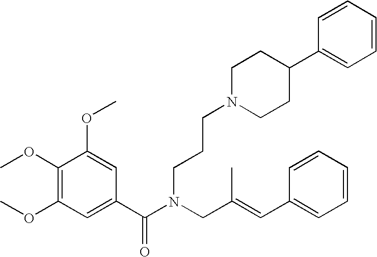 |
| 8 | 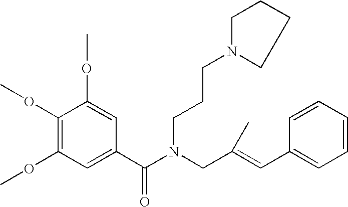 |
| 9 | 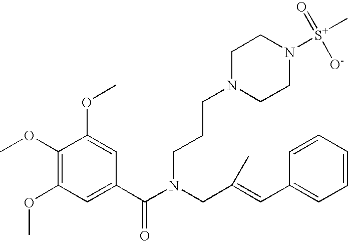 |
| 10 | 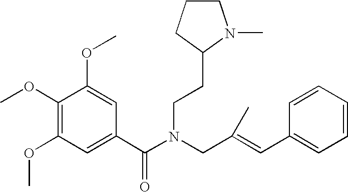 |
| 11 | 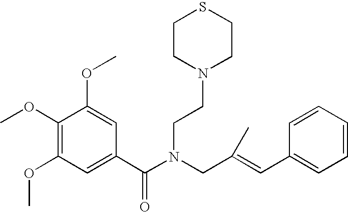 |
| 12 | 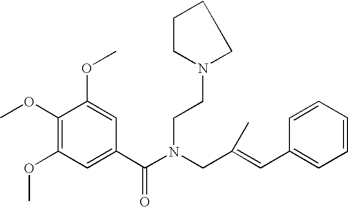 |
| 13 | 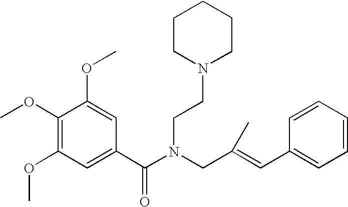 |
| 14 | 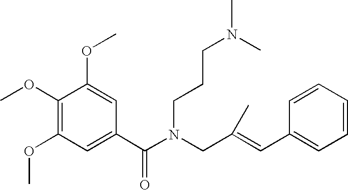 |
| 15 | 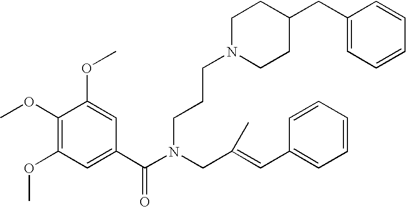 |
| 16 | 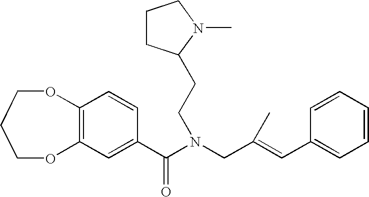 |
| 17 | 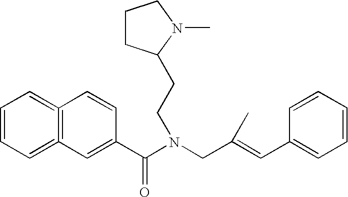 |
| 18 | 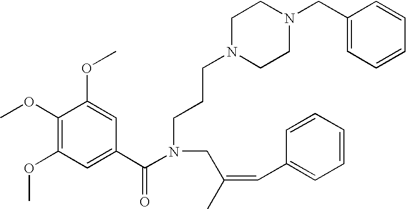 |
| 19 | 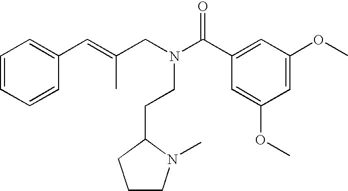 |
| 20 | 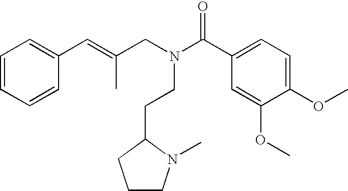 |
| 21 | 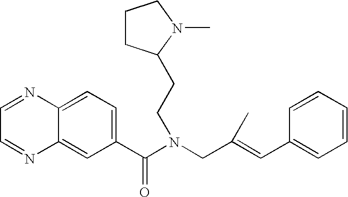 |
| 22 | 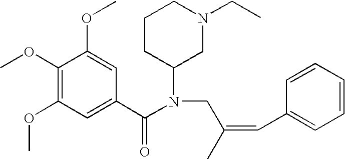 |
| 23 | 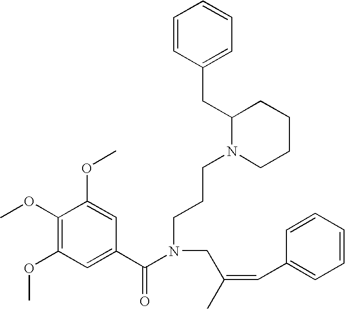 |
| 24 | 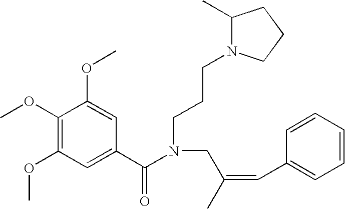 |
| 25 | 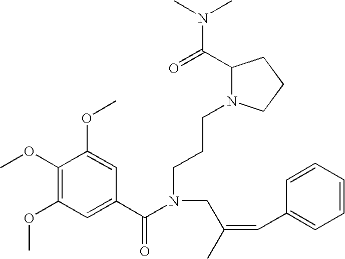 |
| 26 | 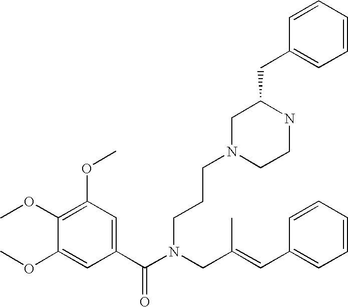 |
| 27 | 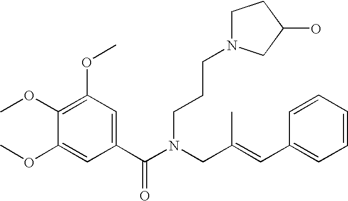 |
| 28 | 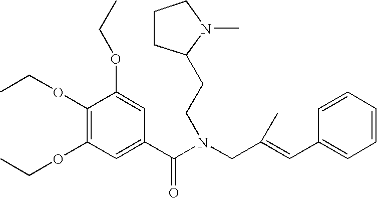 |
| 29 | 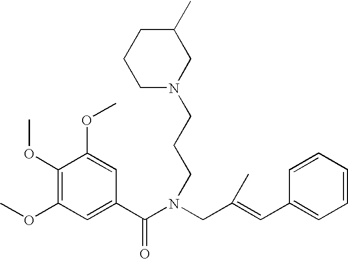 |
| 30 | 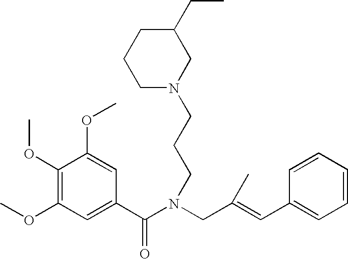 |
| 31 | 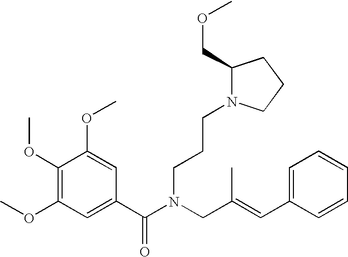 |
| 32 | 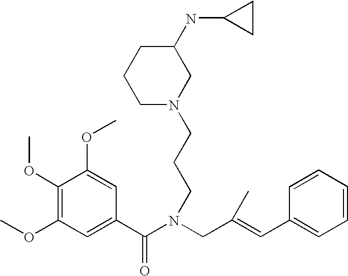 |
| 33 | 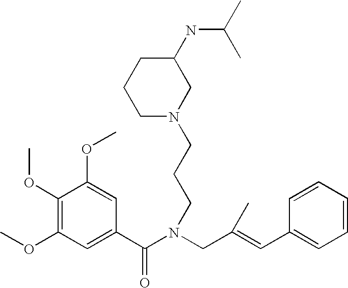 |
| 34 | 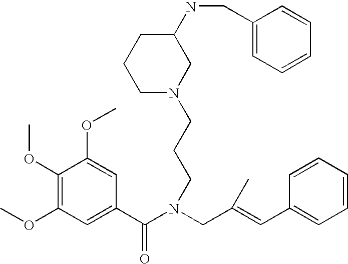 |
| 35 | 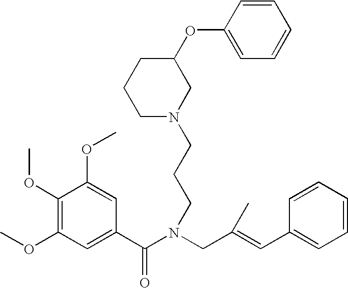 |
| 36 | 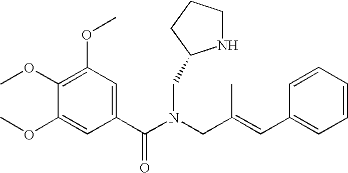 |
| 37 | 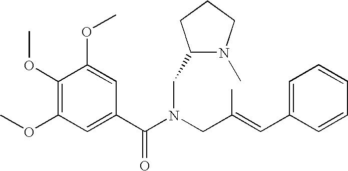 |
| 38 | 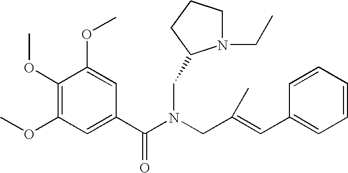 |
| 39 | 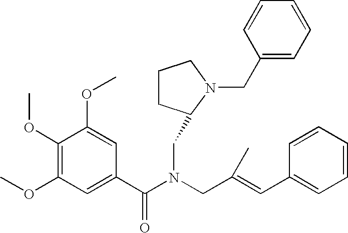 |
| 40 | 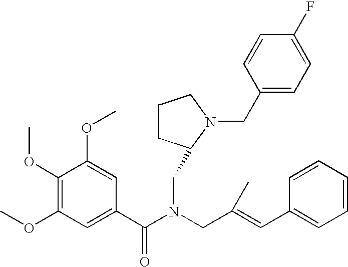 |
| 41 | 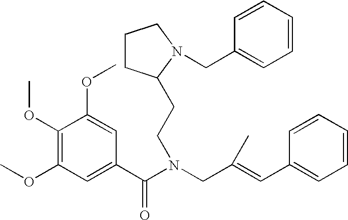 |
| 42 | 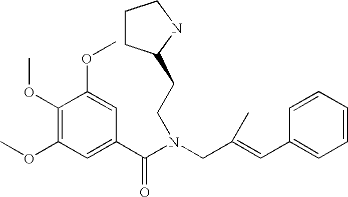 |
| 43 | 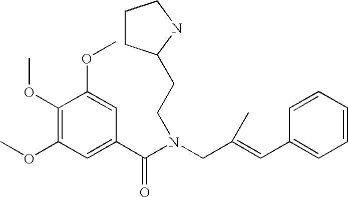 |
| 44 | 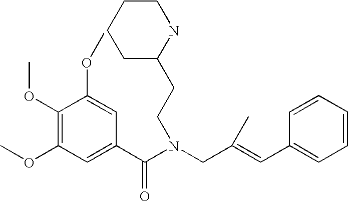 |
| 45 | 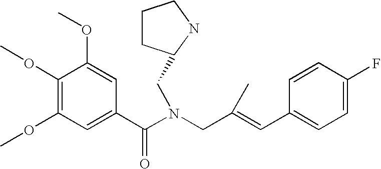 |
| 46 | 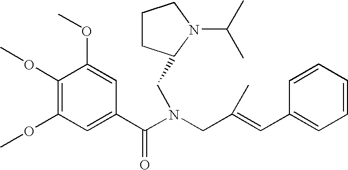 |
| 47 | 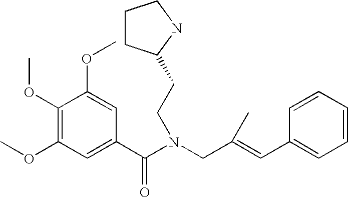 |
| 48 | 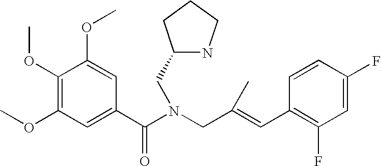 |
| 49 | 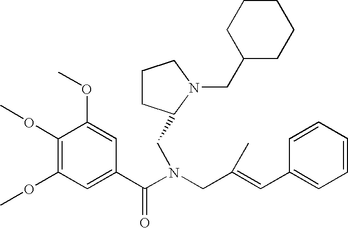 |
| 50 | 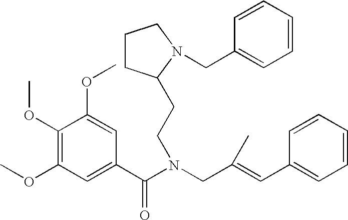 |
| 51 | 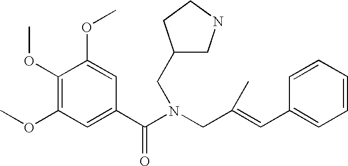 |
| 52 | 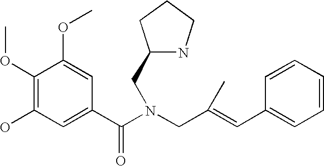 |

TABLE II

| No. | Compound |
|---|---|
| 53 | 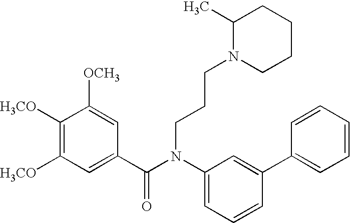 |
| 54 | 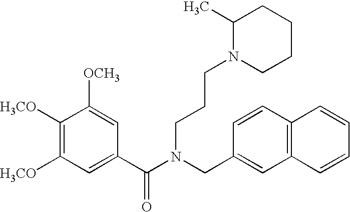 |
| 55 | 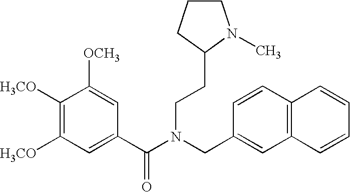 |
| 56 | 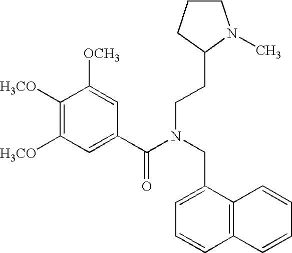 |
| 57 | 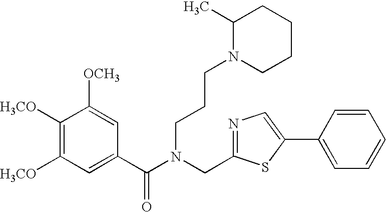 |
| 58 | 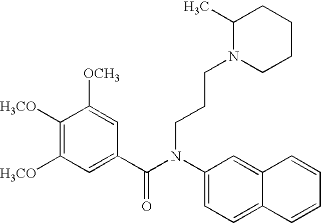 |
| 59 | 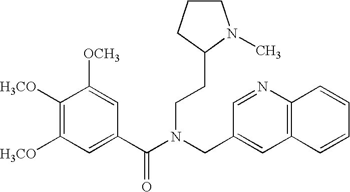 |
| 60 | 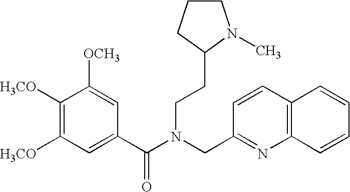 |
| 61 | 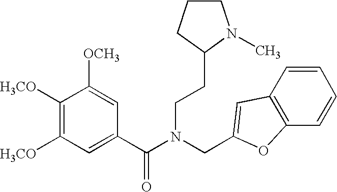 |
| 62 | 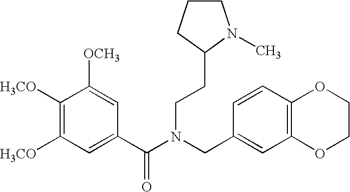 |
| 63 | 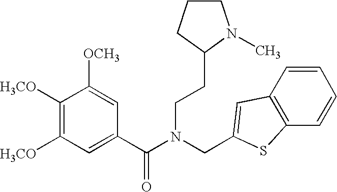 |
| 64 | 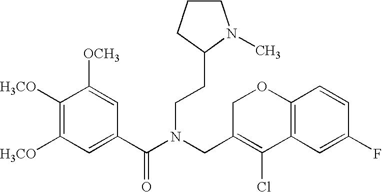 |
| 65 | 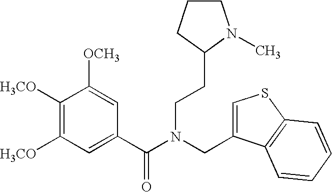 |
| 66 | 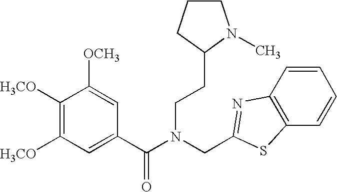 |
| 67 | 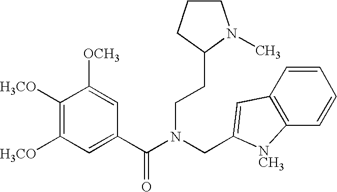 |
| 68 | 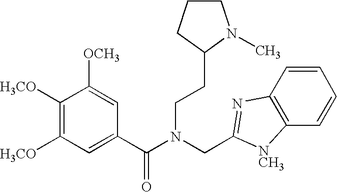 |
| 69 | 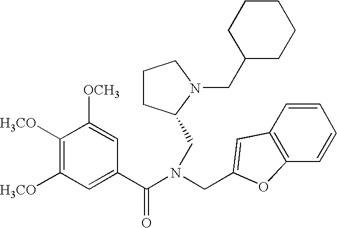 |
| 70 | 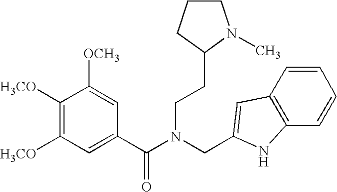 |
| 71 | 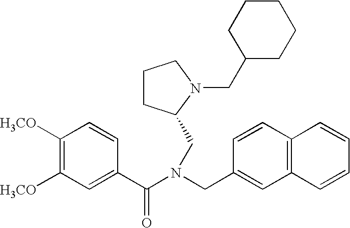 |
| 72 | 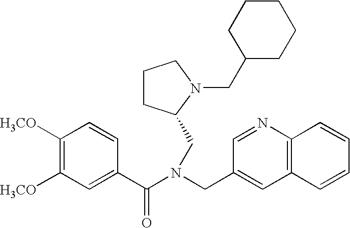 |

Molecule CCX7923 (see, PCT/US02/38555) is commercially available and can be made by the condensation of N-[3-(dimethylamino)propyl]-N,N-dimethyl-1,3-propanediamine with bromomethyl-bicyclo(2,2,1)hept-2-ene by methods known in the art. CCX0803 (see, PCT/US02/38555) is commercially available and can be made by condensation of 3-(2-bromoethyl)-5-phenylmethoxy-indole and 2,4,6-triphenylpyridine by methods well known in the art. See, e.g., Organic Function Group Preparations, 2nd Ed. Vol. 1, (S. R. Sandler & W. Karo 1983); Handbook of Heterocyclic Chemistry (A. R. Katritzky, 1985); Encyclopedia of Chemical Technology, 4th Ed. (J. I. Kroschwitz, 1996). In some embodiments of the invention, CCX7923 is not included as a modulator of CCX-CKR2.

IV. Agonists

Agonists of CCX-CKR2 include naturally-occurring agonists such as SDF-1 and I-TAC as well as antibody, chemokine fragments, peptide mimetics, and small organic molecule agonists. Agonists can be selected using standard library screening, as described herein, to identify molecules that increase CCX-CKR2 activity.

V. Expressing CCX-CKR2 in a Subject

In some embodiments, CCX-CKR2 is expressed in a subject, thereby promoting angiogenesis. In some cases, a polynucleotide encoding CCX-CKR2 is introduced into a cell in vitro and the cell is subsequently introduced into a subject. In some of these cases, the cells are first isolated from the subject and then re-introduced into the subject after the polynucleotide is introduced. In other embodiments, polynucleotides encoding CCX-CKR2 are introduced directly into cells in the subject in vivo.

In some cases, the CCX-CKR2-encoding polypeptides are introduced into cells from: (i) a tissue of interest, (ii) exogenous cells introduced into the tissue, or (iii) neighboring cells not within the tissue. In some embodiments, the polynucleotides of the invention are introduced into endothelial cells. The tissue with which the endothelial cells are associated is any tissue in which it is desired to enhance the migration or expansion of endothelia.

Similarly, polynucleotides can be introduced into cells to inhibit expression of CCX-CKR2. Typically these polynucleotides will include antisense or siRNA constructs designed to inhibit CCX-CKR2 transcription or RNA stability. Such polynucleotides can be delivered by similar means as delivery of CCX-CKR2 polynucleotides.

Conventional viral and non-viral based gene transfer methods can be used to introduce nucleic acids encoding engineered polypeptides of the invention in mammalian cells or target tissues. Such methods can be used to administer nucleic acids encoding polypeptides of the invention (e.g., CCX-CKR2) to cells in vitro. In some embodiments, the nucleic acids encoding polypeptides of the invention are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding engineered polypeptides of the invention take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the polypeptides of the invention is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:15-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA) encoding a polypeptides of the invention, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panb cells), GR-1 (granulocytes), and lad (differentiated antigen presenting cells) (see Inaba et al., J. Exp. Med. 176:1693-1702 (1992)).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

VII Treatment of Diseases and Disorders

A. Increasing Angiogenesis

The present invention contemplates increasing angiogenesis, as described herein, in any subject in need thereof. Increasing angiogenesis can be useful, for example, for healing of wounds, fractures, and burns, as well as treating inflammatory diseases, heart disease, e.g., restenosis, ischeric heart, myocardial infarction and peripheral vascular diseases (e.g., in diabetics). Enhancing angiogenesis can also be useful in, e.g., treating stroke, infertility, scleroderma as well as following microsurgery and re-attachment of limbs, digits, and organs.

B. Decreasing Angiogenesis

The present invention contemplates decreasing angiogenesis, as described herein, in any subject in need thereof. For example, decreasing CCX-CKR2 activity, thereby decreasing angiogenesis, is useful to inhibit formation, growth and/or metastasis of tumors, especially solid tumors. Examples of tumors including carcinomas, adenocarcinomas, lympohomas, sarcomas, and other solid tumors, as described in U.S. Pat. No. 5,945,403, solid tumors; blood born tumors such as leukemias; tumor metastasis; benign tumors, for example hemangiomas, acoustic neuromas, neurofibromas, trachomas, and pyogenic granulomas. In some cases, angiogenesis is reduced according to the methods of the invention in subjects having, e.g., carcinomas, gliomas, mesotheliomas, melanomas, lymphomas, leukemias, adenocarcinomas, breast cancer, ovarian cancer, cervical cancer, glioblastoma, leukemia, lymphoma, prostate cancer, and Burkitt's lymphoma, head and neck cancer, colon cancer, colorectal cancer, non-small cell lung cancer, small cell lung cancer, cancer of the esophagus, stomach cancer, pancreatic cancer, hepatobiliary cancer, cancer of the gallbladder, cancer of the small intestine, rectal cancer, kidney cancer, bladder cancer, prostate cancer, penile cancer, urethral cancer, testicular cancer, cervical cancer, vaginal cancer, uterine cancer, ovarian cancer, thyroid cancer, parathyroid cancer, adrenal cancer, pancreatic endocrine cancer, carcinoid cancer, bone cancer, skin cancer, retinoblastomas, Hodgkin's lymphoma, non-Hodgkin's lymphoma (see, CANCER:PRINCIPLES AND PRACTICE (DeVita, V. T. et al. eds 1997) for additional cancers).

Other disorders involving unwanted or problematic angiogenesis include rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; disease of excessive or abnormal stimulation of endothelial cells, including intestinal adhesions, Crohn's disease, skin diseases such as psoriasis, excema, and scleroderma, diabetes, diabetic retinopathy, retinopathy of prematurity, age-related macular degeneration, atherosclerosis, scleroderma, wound granulation and hypertrophic scars, i.e., keloids, and diseases that have angiogenesis as a pathologic consequence such as cat scratch disease and ulcers (Helicobacter pylori), can also be treated. Angiogenic inhibitors can be used to prevent or inhibit adhesions, especially intra-peritoneal or pelvic adhesions such as those resulting after open or laproscopic surgery, and bum contractions. Other conditions which should be beneficially treated using the angiogenesis inhibitors include prevention of scarring following transplantation, cirrhosis of the liver, pulmonary fibrosis following acute respiratory distress syndrome or other pulmonary fibrosis of the newborn, implantation of temporary prosthetics, and adhesions after surgery between the brain and the dura. Endometriosis, polyposis, cardiac hypertrophyy, as well as obesity, may also be treated by inhibition of angiogenesis. These disorders may involve increases in size or growth of other types of normal tissue, such as uterine fibroids, prostatic hypertrophy, and amyloidosis. Modulators of CCX-CKR2 may be used prophylactically or therapeutically for any of the disorders or diseases described herein.

Decreasing CCX-CKR2 activity can also be used in the prevention of neovascularization to effectively treat a host of disorders. Thus, for example, the decreasing angiogenesis can be used as part of a treatment for disorders of blood vessels (e.g., hemangiomas and capillary proliferation within atherosclerotic plaques), muscle diseases (e.g., myocardial angiogenesis, myocardial infarction or angiogenesis within smooth muscles), joints (e.g., arthritis, hemophiliac joints, etc.), and other disorders associated with angiogenesis. Promotion of angiogenesis can also aid in accelerating various physiological processes and treatment of diseases requiring increased vascularization such as the healing of wounds, fractures, and burns, inflammatory diseases, ischeric heart, and peripheral vascular diseases.

As described in the examples below, antagonists of CCX-CKR2 may also be used to enhance wound healing. Without intending to limit the invention to a particular mechanism of action, it may be that antagonism of CCX-CKR2 allows for endogenous ligands to instead bind to lower affinity receptors, thereby triggering enhanced wound healing. For example, SDF-1 binds to both CCX-CKR2 and CXCR4, but binds to CXCR4 with a lower affinity. Similarly, I-TAC binds to CXCR3 with a lower affinity than I-TAC binds to CCX-CKR2. By preventing binding of these ligands to CCX-CKR2, CCX-CKR2 antagonists may allow the ligands to bind to the other receptors, thereby enhancing wound healing. Thus, the antagonism of CCX-CKR2 to enhance wound healing may be mediated by a different mechanism than enhancing wound healing by stimulating CCX-CKR2 activity with an agonist.

Aside from treating disorders and symptoms associated with neovascularization, the inhibition of angiogenesis can be used to modulate or prevent the occurrence of normal physiological conditions associated with neovascularization. Thus, for example the inventive method can be used as a birth control. In accordance with the present invention, decreasing CCX-CKR2 activity within the ovaries or endometrium can attenuate neovascularization associated with ovulation, implantation of an embryo, placenta formation, etc.

Modulators of angiogenesis have yet other therapeutic uses. Accordingly, the CCX-CKR2 modulators of the present invention may be used for the following:
(a) Adipose tissue ablation and treatment of obesity. See, e.g, Kolonin et al., Nature Medicine 10(6):625-632 (2004);
(b) Treatment of preclampsia. See, e.g., Levine et al., N. Engl. J. Med. 350(7): 672-683 (2004); Maynard, et al., J. Clin. Invest. 111(5): 649-658 (2003); and
(c) Treatment of cardiovascuar disease. See, e.g., March, et al., Am. J. Physiol. Heart Circ. Physiol. 287:H458-H463 (2004); Rehman et al., Circulation 109: 1292-1298 (2004).
   VIII. Administration And Pharmaceutical Compositions

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, subcutaneously, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The composition can be administered by means of an infusion pump, for example, of the type used for delivering insulin or chemotherapy to specific organs or tumors. Compositions of the inventions can be injected using a syringe or catheter directly into a tumor or at the site of a primary tumor prior to or after excision; or systemically following excision of the primary tumor. The compositions of the invention can be administered topically or locally as needed. For prolonged local administration, the enzymes may be administered in a controlled release implant injected at the site of a tumor. For topical treatment of a skin condition, the enzyme formulation may be administered to the skin in an ointment or gel.

The modulators (e.g., agonists or antagonists) of the expression or activity of CCX-CKR2, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

In some embodiments, CCX-CKR2 modulators of the present invention can be administered in combination with other appropriate therapeutic agents, including, e.g., chemotherapeutic agents, radiation, etc. Selection of the appropriate agents for use in combination therapy may be made by one of ordinary skill in the art, according to conventional pharmaceutical principles. The combination of therapeutic agents may act synergistically to effect the treatment or prevention of the various disorders such as, e.g., cancer, wounds, kidney dysfunction, brain dysfunction or neuronal dysfunction. Using this approach, one may be able to achieve therapeutic efficacy with lower dosages of each agent, thus reducing the potential for adverse side effects.

The dose administered to a patient, in the context of the present invention should be sufficient to effect a beneficial response in the subject over time (e.g., to reduce tumor size or tumor load). The optimal dose level for any patient will depend on a variety of factors including the efficacy of the specific modulator employed, the age, body weight, physical activity, and diet of the patient, on a possible combination with other drugs, and on the severity of a particular disease. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, chemokine receptor modulators of the present invention can be administered at a rate determined by the LD-50 of the modulator, and the side-effects of the modulator at various concentrations, as applied to the mass and overall health of the subject. Administration can be accomplished via single or divided doses.

IX. Combination Therapies

Inhibitors of CCX-CKR2 can be supplied alone or in conjunction with one or more other drugs. Possible combination partners can include, e.g., additional anti-angiogenic factors and/or chemotherapeutic agents (e.g., cytotoxic agents) or radiation, a cancer vaccine, an immunomodulatory agent, an anti-vascular agent, a signal transduction inhibitor, an antiproliferative agent, or an apoptosis inducer.

Inhibitors of CCX-CKR2 can be used in conjunction with antibodies and peptides that block integrin engagement, proteins and small molecules that inhibit metalloproteinases (e.g., marmistat), agents that block phosphorylation cascades within endothelial cells (e.g., herbamycin), dominant negative receptors for known inducers of angiogenesis, antibodies against inducers of angiogenesis or other compounds that block their activity (e.g., suramin), or other compounds (e.g., retinoids, IL-4, interferons, etc.) acting by other means. Indeed, as such factors may modulate angiogenesis by different mechanisms, employing inhibitors of CCX-CKR2 in combination with other antiangiogenic agents can potentiate a more potent (and potentially synergistic) inhibition of angiogenesis within the desired tissue.

Anti-angiogenesis agents, such as MMP-2 (matrix-metalloprotienase 2) inhibitors, MMP-9 (matrix-metalloprotienase 9) inhibitors, and COX-II (cyclooxygenase II) inhibitors, can be used in conjunction with inhibitors of CCX-CKR2 and pharmaceutical compositions described herein. Inhibitors of CCX-CKR2 can also be used with signal transduction inhibitors, such as agents that can inhibit EGFR (epidermal growth factor receptor) responses, such as EGFR antibodies, EGF antibodies, and molecules that are EGFR inhibitors; VEGF (vascular endothelial growth factor) inhibitors, such as VEGF receptors and molecules that can inhibit VEGF; and erbB2 receptor inhibitors, such as organic molecules or antibodies that bind to the erbB2 receptor, for example, HERCEPTIN·. (Genentech, Inc. of South San Francisco, Calif., USA).

Molecules that increase or decrease CCX-CKR2 activity can also be combined with other drugs including drugs that promote angiogenesis and/or wound healing. Those of skill in the art will appreciate that one can incorporate one or more medico-surgically useful substances or therapeutic agents, e.g., those which can further intensify the angiogenic response, and/or accelerate and/or beneficially modify the healing process when the composition is applied to the desired site requiring angiogenesis. For example, to further promote angiogenesis, repair and/or tissue growth, at least one of several hormones, growth factors or mitogenic proteins can be included in the composition, e.g., fibroblast growth factor, platelet derived growth factor, macrophage derived growth factor, etc. In addition, antimicrobial agents can be included in the compositions, e.g., antibiotics such as gentamicin sulfate, or erythromycin. Other medico-surgically useful agents can include anti-inflammatories, analgesics, anesthetics, rubifacients, enzymes, antihistamines and dyes.

Molecules that decrease CCX-CKR2 activity can also be combined with other drugs including drugs for treating arthritis. Examples of such agents include anti-inflammatory therapeutic agents. For example, glucocorticosteroids, such as prednisolone and methylprednisolone, are often-used anti-inflammatory drugs. Nonsteroidal anti-inflammatory drugs (NSAIDs) are also used to suppress inflammation. NSAIDs inhibit the cyclooxygenase (COX) enzymes, COX-1 and COX-2, which are central to the production of prostaglandins produced in excess at sites of inflammation. In addition, the inflammation-promoting cytokine, tumor necrosis factor α (TNFα), is associated with multiple inflammatory events, including arthritis, and anti-TNFα therapies are being used clinically.

### EXAMPLES

The following example is provided to illustrate, not limit, the invention.

### Example 1

The SDF-1/CXCR4 chemokine-receptor pair has long been considered to share an exclusive interaction in that SDF-1 has not been reported to function through another receptor and an additional ligand for CXCR4 has not been identified (reviewed in Zlotnik, A., and Yoshie, O., Immunibty 12:121-127 (2000)). This notion is supported by the fact that the genetic knock-outs of both genes result in death of the embryos (Nagasawa, T. et al. Nature 382, 635-638 (1996); Yong-Rui Zou, et al. Natrure 393:595-599 (1998)).

In an effort to evaluate SDF-1 and CXCR4 biology, we undertook to evaluate this receptor ligand pair in several different cell types. Initially we began to evaluate receptor expression on several tumor cell types as determined by FACS analysis. Four commercially available antibodies, clones 12G5, 171, 172 and 173, were tested on multiple cell lines. Interestingly, even though the breast tumor cell lines tested were reported to express CXCR4 and expressed CXCR4 message in our hands the antibodies reacted differently on different cell types. The T cell lines tested, CEM-NKr and Jurkat, along with normal human IL-2 cultured lymphocytes (PBMC) reacted with all for clones. However, two breast tumor lines, MCF-7 and MDA MB-231, did not react with the widely used anti-CXCR4 clone 12G5 and reacted only weakly (in comparison to the T cells) with the other antibodies. Another breast tumor line, MDA MB 435s, was included. This line does not express any CXCR4 mRNA and as expected none of the antibodies recognize CXCR4 on the surface. Thus despite the ability to detect CXCR4 message in MCF-7 and MDA MB 231 cells the anti-CXCR4 antibodies exhibited altered reactivity on these cells as compared to T cells.

We further investigated the SDF-1 receptor expression on these cells by examining the ligand binding profile using our DisplaceMax™ technique (Dairaghi D J, et al, J. Biol Chem, 274(31):21569-21574 (1999)). Using ¹²⁵I SDF-L1α as the signature ligand binding was interrogated with >90 chemokine elements. As expected, only SDF-1 (mouse and human; the human form has two alternatively spliced forms of the protein referred to as SDF-1α and SDF-1β) and the HHV8 encoded chemokine vMIP-II competed for binding with labeled SDF-1 on CEM-NKr. To our surprise, the binding profile on MCF-7 was significantly altered. Not only do SDF-1 and vMIP-II displace SDF-1 tracer, but on these cells, I-TAC (mouse and human) exhibited the ability to compete for binding with SDF-1. We next tested labeled I-TAC as the tracer on these breast tumor cells and produced the same ligand signature. The only reported I-TAC receptor is CXCR3 (Cole K E, et al., J Exp Med. 187(12): 2009-21 (1998)). Interestingly, MIG and IP-10, the two other reported CXCR3 ligands do not displace labeled SDF-1 here. Thus, the binding of SDF-1 to its receptor as expressed on MCF-7 cells differs from that expressed on CEM-NKr in terms of ligand specificity.

Given the altered ligand binding profile to the SDF-1 receptor as expressed on CEM-NKr and MCF-7 we examined the affinity of the ligand for the two cell types. On CEM-NKr homologous competition of ¹²⁵I SDF-1α: with cold SDF-1α or SDF-1β resulted in IC50 values in the low-nM range (1 nM and 1.5 nM respectively). However, in contrast to CEM-NKr, MCF-7 cells exhibited a sub-nM SDF-1 receptor affinity. Furthermore, I-TAC can compete for radiolabeled SDF-1α binding on MCF-7 with low-nM affinity as well. Additionally, we have developed small molecules that inhibit the ability of both SDF-1 and I-TAC to bind specifically to the receptor as expressed on MCF-7 cells and not on CEM-NKr. This series of molecules, referred to as CCX700 and exemplified in Tables I and II, specifically inhibits radiolabeled SDF-1 and I-TAC from binding to MCF-7 cells with low-nM affinity. However the same compound on CEM-NKr has no effect on SDF-1 binding to these cells. Furthermore, a compound (AMD3100) described in the literature to bind to CXCR4, the hallmark SDF-1 receptor, inhibits SDF-1 binding to CEM-NKr but has no effect on SDF-1 binding to MCF-7 cells. Thus, the SDF-1 receptor expressed on MCF-7 cells exhibits altered ligand binding specificity and affinity and this receptor is pharmacologically distinct from the SDF-1 receptor expressed on CEM-NKr.

Given the different properties of the SDF-1 receptor expressed on CEM-NKr as compared to MCF-7 we began to investigate the possibility that this receptor is a discrete gene from CXCR4. After searching the literature and evaluating several orphan GPCR we finally identified the gene responsible for the novel SDF-1 binding characteristics. This gene, referred to as CCX-CKR2, when expressed in a cell line (MDA MB 435s) that does not endogenously express CXCR4 or CCX-CKR2, recapitulates the hallmark binding profiles we had previously detected. In the CCX-CKR2 MDA MB 435s cells, ¹²⁵I SDF-1 binds and SDF-1 and I-TAC compete with sub-nM and low-nM affinity respectively. Additionally, our CCX-CKR2 antagonist series CCX700 (as exemplified in Table I and II) can compete for binding on these cells, however, the widely used CXCR4 antagonist from AnorMed does not affect ¹²⁵I SDF-1 binding on these cells. Thus, the binding anomalies we had detected in MCF-7 cells as compared to CEM-NKr are explained by an additional SDF-1 receptor identified here as a discrete gene called CCX-CKR2.

Using the radioligand binding assay as a diagnostic we have identified multiple tumor cell types which express CCX-CKR2. These cell types are listed in Table 3. Interestingly, this receptor appears to be preferentially expressed on tumor cells over normal cells with few exceptions. Using mouse organs as a source of normal cells we are unable to detect CCX-CKR2 expression on all organ homogenates tested expect normal adult kidney, normal adult brain and certain stages of fetal liver. The expression levels on adult kidney and brain are low as determined by the radio-ligand binding signal. By contrast this receptor is highly expressed in fetal liver at day 11 through 13 of embryonic development. However, by E15 it is gone and cannot be detected at E17 either. Thus, CCX-CKR2 is preferentially expressed in fetal liver during development and then again in tumor cells.

TABLE 3
CCX-CKR2 is Widely Expressed on Tumors, but Not on Normal Cells

| CCX-CKR2positive | CCX-CKR2 negative |
|---|---|
| human Mammary Carcinoma (MCF-7, MDA MB 361) | normal human PBMC |
| human Glioblastoma (T98G) | human T cell leukemia (MOLT4, Jurkat, CEM-NKr) |
| human Prostate Carcinoma (LN Cap) | unstimulated endothelial cells |
| human B cell Lymphoma (Raji, IM9) | mouse thymus |
| human Ovarian Carcinoma (HeLa) | mouse lung |
| human Lung Carcinoma (A549) | mouse heart |
| mouse Mammary carcimoma (4T1) | mouse PBL |
| mouse Pancreatic Epithelial cells, SV40 transformed (SVR) | mouse liver |
| mouse B cell Lymphoma (BCL1) | mouse total adult bone marrow |
| mouse normal kidney* | mouse lineage negative adult bone marrow |
| mouse normal brain* | mouse fetal liver (E15 through birth) |
| mouse fetal liver (E11 through E13) | |
| activated endothelial cells | |
| mouse spleen* | |

*expression on these organs is weak as determined by radioligand binding signal

We also determined that CCX-CKR2 is involved in angiogenesis in the zebrafish morpholino model (Nasevicius A, and Ekker S. C. Nature Genetics 26:216-220 (2000); Ekker S. and Larson J. D. Genesis 30:89-93 (2001)). Zebrafish have been used to evaluate the function of genes involved in early development. Greater than 90% of genes in humans have the same function in zebrafish. Zebrafish have an ortholog of CCX-CKR2 that is 59% identical to the human CCX-CKR2 protein sequence. Using morpholino technology the zebrafish homolog was ‘knocked down’ in developing embryos. Inhibition of the CCX-CKR2 gene with morpholinos prevents early development and most embryos die. However, by titration of the dose of inhibitor the effects of blocking CCX-CKR2 begin to emerge. Interestingly, in the embryos in which CCX-CKR2 is inhibited the morphants exhibit elongation suggesting the cells are unable to partition into dorsal and ventral regions at an early stage (9 hours post fertilization). Further along in development (28 hours post fertilization) the fish have a dorsalization phenotype and vascular defects. By 56 hours post fertilization, the morphants exhibit pericardial edema. In conclusion, the inhibition of CCX-CKR2 in early zebrafish development results in a mild dorsalization phenotype and a dramatic vascular defect. Zebrafish that do not have CCX-CKR2 during development do not develop a vascular system and while they do have blood this blood cannot circulate.

CCX-CKR2 has also been evaluated in a xenograft model of human B cell lymphoma. In this model immunodeficient mice were inoculated with the human B cell lymphoma, NAMALWA. Mice were given either a compound from the CCX700 series or the vehicle control daily. Interestingly, mice receiving the vehicle preferentially developed large, encapsulated, vascularized tumors while mice receiving CCX700 had tumors but the tumors had greatly reduced vascularization and were not encapsulated. This observation is in line with the results from the zebrafish studies in that an inhibitor of CCX-CKR2 inhibited the ability of the tumor to develop a vascular bed.

Inhibitors of CCX-CKR2 were also effective in reducing tumor volumes in a syngeneic lung carcinoma mouse model.

### Example 2

The ability of cells expressing CCX-CKR2 to adhere to an endothelial monolayer has been evaluated in an in vitro static adhesion assay. HUVEC cells (Clontech, Calif.) were adhered to 24 well plastic tissue plates overnight at a density of 100,000 cells/per well. Cells were then treated with medium containing 10 ng/ml TNF-alpha plus 10 ng/ml of IL-1beta or medium alone for 5 hours at 37C. MDA MB 435s (ATCC, VA) wild type or CCX-CKR2 stably transfected cells were loaded with 3 ng/ml calcein AM (Neuroprobes, Oreg.) in PBS for 30 minutes at room temperature. After the incubation cells were washed in PBS and resuspended in HBSS (Hank's buffered saline solution) to a density of 200,000 cells/well. The MDA MB 435s wild type or CCX-CKR2-expressing cells were then added to tissue culture plates containing HUVEC, in duplicate wells. Plates were incubated at 37° C. for 15 minutes and washed twice with HBSS.

Adherent cells were quantified by microscopy and by fluorescence intensity on a TECAN multi-well plate reader. In wells containing unstimulated HU VEC, very few MDA MB 435s cells (wild type or CCX-CKR2) bound to the endothelial layer. However, in wells in which the HU VEC monolayer had been stimulated with TNFalpha and IL-1beta, significantly more of the CCX-CKR2 expressing cells adhered to the monolayer as compared to the wild type, non-CCX-CKR2 expressing cells. When quantified by fluorescence intensity the wells containing CCX-CKR2-expressing MDA MB 435s cells with the activated HU VEC monolayer gave a signal that was four times that of the wells containing the wild type cells and the activated HU VEC monolayer. These data demonstrate that CCX-CKR2 is involved in adhesion to activated endothelial monolayer.

### Example 3

This example demonstrates the efficacy of a CCX-CKR2 ligand competitor in mouse wound healing model.

Wound healing is typically divided into three phases. The first phase, known as the inflammatory phase involves hemostasis and inflammation. The next phase, referred to as the proliferative phase, is characterized by epithelialization, angiogenesis and granulation tissue formation. Finally in the maturational phase the wound contracts and collagen is deposited. It is generally in the proliferative phase during wound angiogenesis that agents effecting angiogenesis have effects on this process.

We have linked CCX-CKR2 to angiogenesis regulation through the zebrafish studies discussed above. Given these results we tested a compound specific for CCX-CKR2 in a model of wound healing.

In the wound healing studies, ICR derived male mice (24±2 g) were used. During the testing period, the animals were singly housed in individual cages. Under hexobarbital (90 mg/kg, IP) anesthesia, the shoulder and back region of each animal was shaved. A sharp punch (ID 12 mm) was applied to remove the skin including panniculus carnosus and adherent tissues. A test compound previously shown to compete with SDF-a and I-TAC for binding to CCX-CKR2 (700 series compound, 100 μg/mouse) and the vehicle (0.5% CMC (carboxymethylcellulose)/PBS pH 7.4, 20 μl/mouse) were each administered topically immediately following cutaneous injury once daily for 10 consecutive days. The positive control, an A2 adenosine receptor agonist (CGS-21680; 10 μg/mouse), was also administered topically daily over the course of the experiment. The wound area, traced onto clear plastic sheets, was measured by use of an Image Analyzer (Life Science Resources Vista, Version 3.0) on days 1, 3, 5, 7, 9 and 11. The percent closure of the wound (%) was calculated, and wound half-closure time (CT50) was determined and analyzed by linear regression using Graph-Pad Prism (Graph Pad Software USA). Unpaired Student's t test was applied for comparison between the treated and vehicle groups at each measurement time point. Differences were considered of statistical significance at P<0.05 level.

Treatment with the 700 series compound in this model promoted wound closure. The 700 series compound at 100 μg/mouse for 10 days significantly increased (P<0.05) wound closure on days 3, 5, 7, 9 and 11, with decreased CT50, relative to corresponding vehicle control values. We also included a known CXCR4 antagonist, AMD3100, to examine any contribution to wound healing by the other known SDF-1/CXCL12 receptor. By comparison, the CXCR4 antagonist (100 μg/mouse) did not cause significant increase (P<0.05) in wound closure (%) or CT50 relative to the vehicle control group. Thus, the 700 series compound, but not AMD3100, demonstrated significant wound healing activity in the mouse cutaneous wound assay.

We next examined the effects of a dose range of the 700 series compound. We again found that the 700 series compound significantly enhanced wound closure (as compared to vehicle control) and this effect was present at all doses tested. Interestingly, the enhancement of wound closure is strongest with the intermediate doses tested (100 g and 25 μg) and weaker with the highest (250 μg) and lowest (5 μg) doses. Thus, the 700 series compound appears to have a ‘U-shaped’ dose response, consistent with other reported angiogenic therapeutics.

While many cell types are involved in wound healing, such as platelets, neutrophils, leukocytes, macrophages, fibroblasts and keratinocytes to name a few, we have not examined CCX-CKR2 expression specifically on all of these cell types. Certainly the epithelium plays a role in wound healing as well. We have demonstrated that the inflammatory cytokines TNFα and IL-1β do upregulate CCX-CKR2 on multiple types of primary endothelial cells. Therefore, without intending to limit the scope of the present invention, it is possible that effects of CCX-CKR2 specific compounds could be acting upon the activated endothelium or another yet to be determined population of cells.

### Example 4

This example demonstrates that CCX-CKR2 promotes cell survival by reducing apoptosis.

Interactions between chemokines and chemokine receptors are typically assessed by measuring intracellular calcium mobilization and chemotaxis. However, CCX-CKR2 does not produce a transient calcium mobilization or cause cells to migrate in response to its ligands CXCL12 or CXCL11. Cells expressing CCX-CKR2 do however exhibit increased adhesion to activated endothelial cell monolayers. Furthermore, under conditions of low serum supplementation of the culture medium (i.e. 1% instead of the regular 10%), the recovery of live adherent cells after three days was much greater for CCX-CKR2-MDA MB 435s transfectants (designated CCX-CKR2 435s) versus untransfected WT cells (WT 435s). Consistent with this observation, the frequency of dead cells recovered in the supernatant collected from these cultures was much greater for WT versus CCX-CKR2-transfectants. This effect could be visualized fluorescently using the DNA intercalating dye 7AAD (7 aminoactinomycin D). CCX-CKR2-435s transfectants or wild type 435s cells were grown in different serum concentrations, then harvested and incubated with 7AAD (1 ug/ml in DMSO) for 15-30 minutes at room temperature. FACS analysis revealed many more dead/apoptotic cells (i.e. 7AAD-positive) in wild-type 435s cells versus CCX-CKR2-435s transfectants.

We have now extended these findings in a series of experiments where cultured CCX-CKR2-transfectants or untransfected WT cells are co-stained with Annexin which detects only apoptotic cells, and propridium iodide (PI) which detects dead cells but not apoptotic cells. This approach readily identifies the proportion of apoptotic cells in a cell population, as demonstrated using agents known to induce cellular apoptosis, e.g. camptothecin (CMP), or TNFalpha plus cycloheximide (CHX), and which provide excellent controls in these assays.

Using this assay, we measured the development of apoptotic cells over time of CCX-CKR2-435s transfectants or wild type 435s cells grown either in optimal (10%) or limiting (1%) serum. Both cell types grown in 10% serum showed excellent viability over a 4 day culture period. In contrast, WT cells grown in 1% serum showed a dramatic reduction in viable cells after 3 and 4 days of culture. Co-staining with Annexin and PI revealed this reflected development of both apopotoic and dead cells. Interestingly, CCX-CKR2-435s cells grown in 1% serum showed excellent viability over the same 4 day culture period, suggesting that the introduction of CCX-CKR2 into 435s protected these cells from the rapid cellular apoptosis occurring under conditions of sub-optimal serum supplementation.

Identical results were obtained in a second experiment using the same CCX-CKR2-435s transfectant, and in addition a separate non-clonal population of 435s cells transfected with CCX-CKR2. The latter results indicated that the apoptosis-sparing property of the initial clonal transfectant resulted from CCX-CKR2 expression rather than a particular aberration of that one transfectant clone.

Complementary data has been obtained using antagonists of CCX-CKR2. In these experiments, addition of CCX-CKR2 antagonists to normal cells had no effect, but addition to the antagonists to cells expressing CCX-CKR2 induced cell death in a dose-dependent manner.

### Example 5

This example demonstrates that cellular expression of CCX-CKR2 causes induction of numerous regulatory proteins.

As an alternative approach to investigating CCX-CKR2-mediated signalling events, supernatants collected from CCX-CKR2 transfected MDA MB 435s cells were compared to supernatants collected from wild-type MDA MB 435s (435s) cells, evaluated by specific ELISA assays for the presence of a large family of secreted proteins. 435s cells expressing CCX-CKR2 produced substantially greater quantities of GM-CSF, RANTES, MCP-1, TIMP-1, and MMP3 than wild-type 435s cells, especially when grown under limiting serum conditions. Interestingly, all these factors have been reported to be involved in growth, vascular remodelling and chemotaxis related to tumorigenesis. They may also be involved in the apoptosis-sparing phenotype of CCX-CKR2 described above.

### Example 6

This example demonstrates that siRNA-based inhibition of CCX-CKR2.

We obtained SMARTpool™ siRNA (Dharmacon) specific for either CXCR4 or CCX-CKR2. SMARTpool™ siRNA is a pool of four different siRNA sequences, each targeting a different region of the specified mRNA. These siRNA pools were tested in HeLa cells. CXCR4 expression was assessed by 12G5 or 173 Mab staining and FACS, while CCX-CKR2 expression was measured in a binding assay using ¹²⁵I-SDF1. CXCR4 is expressed on HeLa cells in a conformation that does not exhibit detectable ¹²⁵I-SDF1 binding, thus allowing for detection of CCX-CKR2 expression. CCX-CKR2 SMARTpool™ siRNA (25-100 nM) effected significant (≧50%) inhibition of ¹²⁵I-SDF1 binding, while CXCR4 SMARTpool™ siRNA did not. Similar results were obtained with 293-CCX-CKR2 transfectants.

In addition, the following 3 siRNA sequences were each found to reduce SDF-1 binding when introduced into cells at a concentration as low as 4 nM:

siRNA #1: GCCGTTCCCTTCTCCATTATT
siRNA #2: GAGCTCACGTGCAAAGTCATT
siRNA #3: GACATCAGCTGGCCATGCATT

The following hairpin siRNAs, based on the mouse CCX-CKR2 transcript, reduce SDF-1 binding via inhibition of murine CCX-CKR2 expression:

5′-CACCGCCTAACAAGAACGTGCTTCTCGAAAGAAGCACGTTC
TTGTTAGGC
5′-CACCGGGTGAATATCCAGGCTAAGACGAATCTTAGCCTGGA
TATTCACCC
5′-CACCGGTCAGTCTCGTGCAGCATAACGAATTATGCTGCACG
AGACTGACC
5′-CACCGCTTCCAACAATGAGACCTACCGAAGTAGGTCTCATT
GTTGGAAGC
5′-CACCGCTGGAGAATGTGCTCTTTACCGAAGTAAAGAGCACA
TTCTCCAGC

### Example 7

This example demonstrates that inhibition of CCX-CKR2 is an effective treatment of arthritis.

The efficacy of compounds that inhibit CCX-CKR2 activity compared to Enbrel® was determined in a rat model of arthritis. Rats received subcutaneous administrations of 700 series CCX-CKR2 binding molecules. The developing type II collagen arthritis rats were monitored for inhibition of inflammation (joint swelling), cartilage destruction and bone resorption.

Female Lewis rats weighing 125-150 g were used. Agents were delivered in vehicle, i.e., Type II collagen and Freund's incomplete adjuvant. Animals (10/group for arthritis, 4/group for normal control), were housed 4-5/cage and were acclimated for 4-8 days after arrival to the animal facility.

Dosing was initiated on day 0 and continued through day 16. Acclimated animals were anesthetized with Isoflurane and given collagen injections (D0). On day 6, they were anesthetized again for a second collagen injection. Collagen was prepared by making a 4 mg/ml solution in 0.01N Acetic acid. Equal volumes of collagen and Freund's incomplete adjuvant were emulsified by hand mixing until a bead of this material held its form when placed in water. Each animal received 300 μl of the mixture each time spread over 3 sites on back.

Calipering of normal (pre-disease) right and left ankle joints were performed on day 9. On days 10-14, onset of arthritis occurred.

Rats were weighed on days 0,3,6,9,10,11,12,13,14,15,16 and 17 of the study and caliper measurements of ankles were taken every day beginning on day 9. Final body weights were taken on day 17. After final body weight measurement, animals were anesthetized for terminal serum collection approximately 24 hrs post dosing (on day 17) and then euthanized and tissues were collected. Knees were also collected into formalin for microscopy.

Following 1-2 days in fixative and then 4-5 days in decalcifier, the ankle joints were cut in half longitudinally, knees were cut in half in the frontal plane, processed, embedded, sectioned and stained with toluidine blue. Collagen arthritic ankles and knees were given scores of 0-5 for inflammation, pannus formation and bone resorption according to the following criteria:

Knee and Ankle Inflammation

0=Normal
1=Minimal infiltration of inflammatory cells in periarticular tissue
2=Mild infiltration
3=Moderate infiltration with moderate edema
4=Marked infiltration with marked edema
5=Severe infiltration with severe edema
   Ankle Pannus (Emphasis on Tibiotarsal Joint)
0=Normal
1=Minimal infiltration of pannus in cartilage and subchondral a bone
2=Mild infiltration (<¼ of tibia at edges)
3=Moderate infiltration (¼ to ⅓ of tibia affected, smaller tarsals affected)
4=Marked infiltration (½-¾ of tibia affected, destruction of smaller tarsals))
5=Severe infiltration (>¾ of tibia affected, severe distortion of overall architecture)
   Knee Pannus
0=Normal
1=Minimal infiltration of pannus in cartilage and subchondral bone
2=Mild infiltration (extends over up to ¼ of surface or subchondral area of tibia or femur)
3=Moderate infiltration (extends over >¼ but <½ of surface or subchondral area of tibia or femur)
4=Marked infiltration (extends over ½ to ¾ of tibial or femoral surface)
5=Severe infiltration (covers >¾ of surface)
   Cartilage Damage (Ankle)
0=Normal
1=Minimal=minimal to mild loss of toluidine blue staining with no obvious chondrocyte loss or collagen disruption
2=Mild=mild loss of toluidine blue staining with focal mild (superficial) chondrocyte loss and/or collagen disruption and full destruction of tibia <¼ of surface, mild changes in smaller tarsals
3=Moderate=moderate loss of toluidine blue staining with multifocal moderate (depth to middle zone) chondrocyte loss and/or collagen disruption, ¼ to ⅓ of tibia affected by full thickness destruction, smaller tarsals affected to ½-¾ depth
4=Marked=marked loss of toluidine blue staining with multifocal marked (depth to deep zone) chondrocyte loss and/or collagen disruption, ½-¾ of tibia with full thickness destruction, destruction of smaller tarsals
5=Severe=severe diffuse loss of toluidine blue staining with multifocal severe (depth to tide mark) chondrocyte loss and/or collagen disruption
   Cartilage Damage (Knee, Emphasis on Femoral Condyles)
0=Normal
1=Minimal=minimal to mild loss of toluidine blue staining with no obvious chondrocyte loss or collagen disruption
2=Mild=mild loss of toluidine blue staining with focal mild (superficial) chondrocyte loss and/or collagen disruption
3=Moderate=Moderate loss of toluidine blue staining with multifocal to diffuse moderate (depth to middle zone) chondrocyte loss and/or collagen disruption
4=Marked=marked loss of toluidine blue staining with multifocal to diffuse marked (depth to deep zone) chondrocyte loss and/or collagen disruption, 5=Severe=severe diffuse loss of toluidine blue staining with multifocal severe (depth to tide mark) chondrocyte loss and/or collagen disruption on both femur and tibia
   Bone Resorption (Ankle)
0=Normal
1=Minimal=small areas of resorption, not readily apparent on low magnification, rare osteoclasts
2=Mild=more numerous areas of resorption, not readily apparent on low magnification, osteoclasts more numerous, <¼ of tibia at edges is resorbed
3=Moderate=obvious resorption of medullary trabecular and cortical bone without full thickness defects in cortex, loss of some medullary trabeculae, lesion apparent on low magnification, osteoclasts more numerous, ¼ to ⅓ of tibia affected, smaller tarsals affected
4=Marked=Full thickness defects in cortical bone, often with distortion of profile of remaining cortical surface, marked loss of medullary bone, numerous osteoclasts, ½-¾ of tibia affected, destruction of smaller tarsals
5=Severe=Full thickness defects in cortical bone, often with distortion of profile of remaining cortical surface, marked loss of medullary bone, numerous osteoclasts, >¾ of tibia affected, severe distortion of overall architecture
   Bone Resorption (Knee)
0=Normal
1=Minimal=small areas of resorption, not readily apparent on low magnification, rare osteoclasts
2=Mild=More numerous areas of resorption, definite loss of subchondral bone involving ¼ of tibial or femoral surface (medial or lateral)
3=Moderate=obvious resorption of subchondral bone involving >¼ but <½ of tibial or femoral surface (medial or lateral)
4=Marked=obvious resorption of subchondral bone involving ≧ 1/22 but <¾ of tibial or femoral surface (medial or lateral)
5=Severe=distortion of entire joint due to destruction involving >¾ of tibial or femoral surface (medial or lateral)

Clinical data for ankle joint diameter was analyzed by determining the area under the dosing curve (AUC). For calculation of AUC, the daily measurement of ankle joints (using a caliper) for each rat were entered into Microsoft Excel and the area between the treatment days after the onset of disease to the termination day was computed. Means for each group were determined and percent inhibition from arthritis controls was calculated by comparing values for treated and normal animals. Data was analyzed by the Student's t-test. Paw weights and histologic parameters (mean±SE) for each group was also analyzed for differences using the Student's t-test. Percent inhibition of paw weight and AUC was calculated using the following formula:
% Inhibition=A−B/A×100
A=Mean Disease Control—Mean Normal
B=Mean Treated—Mean Normal

Rats were treated as follows:

| Group N | Treatment, sc bid, or qd days 0-16 |
|---|---|
| 1 | Normal controls, vehicle sc qd (2 ml/kg) |
| 2 | Arthritis + vehicle sc qd (2 ml/kg) |
| 3 | Arthritis + CCX754 100 mg/kg sc qd |

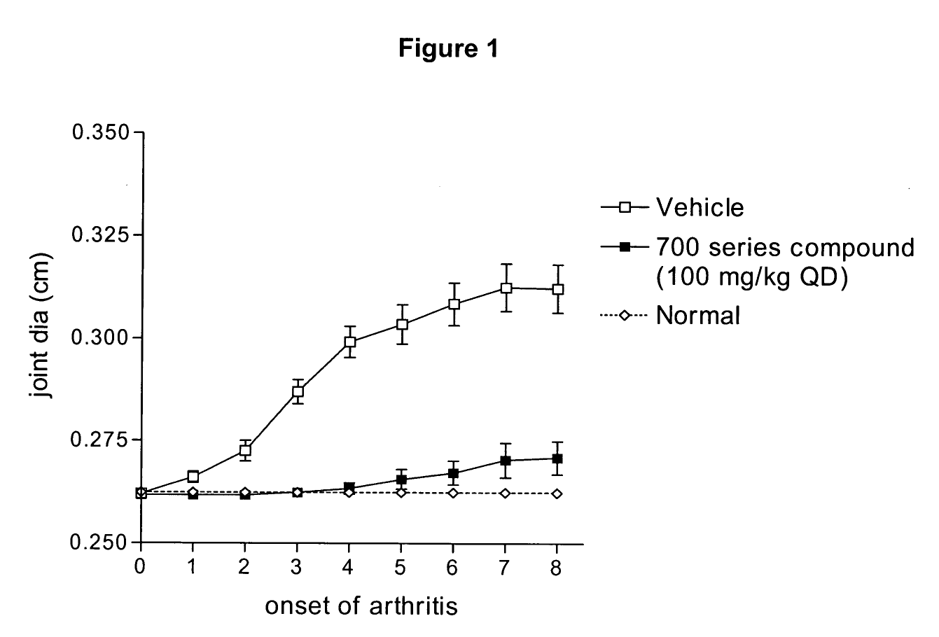

Animals within the group receiving the 700 series compound exhibited significantly reduced joint inflammation as compared to the vehicle treated group (P<0.0001). See, FIG. 1. Rats in groups developing arthritis (vehicle group) exhibited a decrease in body weight over the course of the study while rats with no arthritis (normal controls) or minimal inflammation (700 series treated) exhibited increasing or stabilized body weight, respectively.

### Example 8

This example illustrates the preparation of N-(S)-(1-Cyclohexylmethyl-pyrrolidine-2-ylmethyl)-3,4-dimethoxy-N-naphthalen-2-ylmethyl-benzamide.

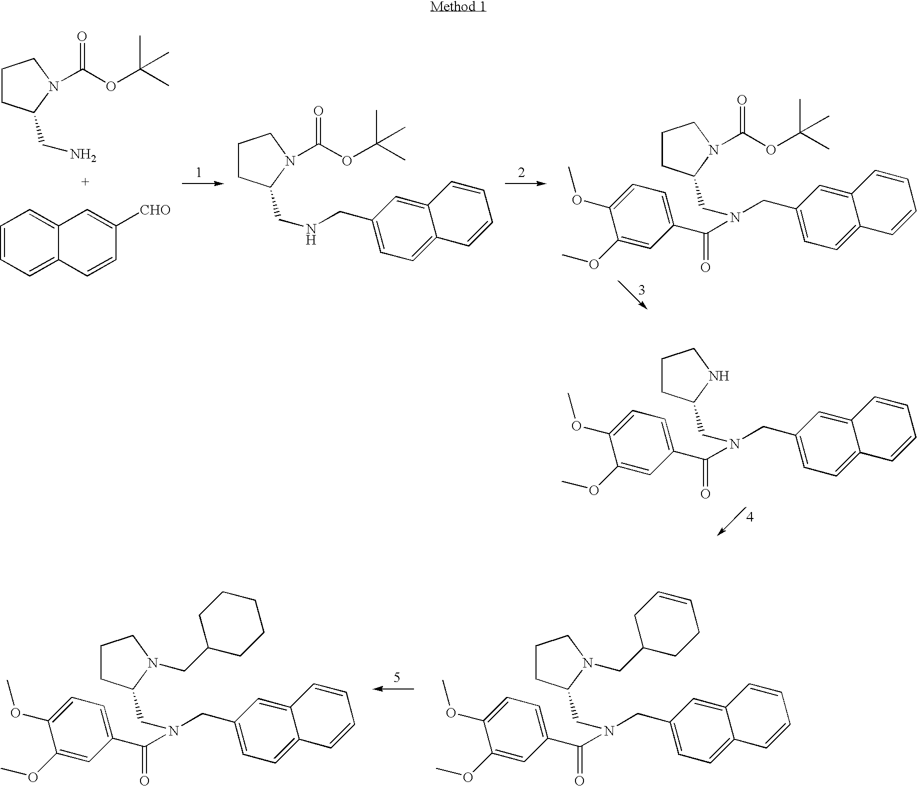

Step 1: (S)-2-{[Naphthalen-2-ylmethyl]-amino]-methyl}-pyrrolidin-1-carboxylic acid tert-butyl ester.

Under nitrogen, 2-(S)-aminomethyl-pyrrolidin-1-carboxylic acid tert-butyl ester (prepared according to the scheme 1) 2 g (10 mmol) was dissolved in 50 mL anhydrous dichloromethane. To this solution was added naphthalene-2-carbaldehyde 2 g (13 mmol), and molecular sieves. The mixture was stirred overnight. Molecular sieves were filtered and the organic portion was concentrated. The resulting mixture was taken up in 100 mL methanol cooled at 0° C., and sodium borohydride 0.75 g (20 mmol) was added. After 1 hour, thin layer chromatography showed the completion of reaction. To this mixture was added very slowly 10 mL of water, and was extract with dichloromethane 3 times, combined organic layer was washed with brine, dried over magnesium sulfate, filtered and concentrated, gave 2.76 g orange oil (no purification). LC-MSD, m/z for: C₂₁H₂₈N₂O₂ [M+H]: 341.1. LC retention time on HPLC, C18 column gradient 20-95% acetonitrile-0.1% TFA in 7 minutes: 3.2 min.

Step 2: 2-(S)-{[(3,4-Dimethoxy-benzoyl)-naphthalen-2-ylmethyl-amino]-methyl}-pyrrolidine-1-carboxylic acid tert-butyl ester.

3,4-Dimethoxy benzoic acid 1.04 g (5.72 mmol) was dissolved in 30 mL tetrahydrofuran, to this mixture was added 1-ethyl-3-(3-dimethylaminopropyl) carbodiimide hydrochloride 1.4 g (6.6 mmol), triethylamine 0.66 g (5.72 mmol), after 30 minutes 1-hydroxybenzotriazole 0.77 g (5.72 mmol) was added. The mixture was stirred one hour. To this mixture was added 2-{[naphthalen-2-ylmethyl]-amino]-methyl}-pyrrolidin-1-carboxylic acid tert-butyl ester 1.5 g (4.4 mmol). The mixture was stirred 1 night at room temperature. Added 50 mL of saturated sodium bicarbonate and extract with ethyl acetate 3 times 100 mL. The combined organic layer was dried over magnesium sulfate, filtered, and concentrated under vacuum. Purification over silica gel hexane:1-dichloromethane: 1, lead to 1.1 g white powder. LC-MSD, m/z for: C₃₀H₃₆N₂O₅ [M+H]: 505.2. LC retention time on HPLC, C18 column gradient 20-95% acetonitrile with 0.1% TFA in 7 minutes: 5.0 min.

Step 3 (S)-3,4-Dimethoxy-N-naphthalen-2ylmethyl-N-pyrrolidin-2-ylmethyl-benzamide.

In 20 mL mixture of dichloromethane and trifluoroacetic acid 30%, was dissolved 2-{[(3,4-Dimethoxy-benzoyl)-naphthalen-2-ylmethyl-amino]-methyl}-pyrrolidine-1-carboxylic acid tert-butyl ester 1.1 g (2.18 mmol). After 1 hour at room temperature, saturated solution of sodium bicarbonate was added until basic pH, the mixture was extracted with dichlorometane, dried over magnesium sulfate, filtered and concentrated under vacuum, yield to 0.88 g. LC-MSD, m/z for: C₂₅H₂₈N₂O₃ [M+H]: 404.2. LC retention time on HPLC, C18 column gradient 20-95% acetonitrile with 0.1% TFA in 7 minutes: 1.37 mm.

Step 4: N-(S)-(1-Cyclohex-3-enylmethyl-pyrrolidin-2-ylmethyl)-3,4-dimethoxy-N-naphthalen-2-ylmethyl-benzamide.

3,4-Dimethoxy-N-naphthalen-2-ylmethyl-N-(S)-pyrrolidin-2-ylmethyl-benzamide 0.88 g (2.17 mmol) was dissolved in 20 mL anhydrous dichloromethane, to this mixture was added 1,2,3,6-tetrahydrobenzaldehyde 0.26 g (2.39 mmol), sodium triacethoxyborohydride 0.68 g (3.25 mmol), and molecular sieve. The reaction mixture was stirred under nitrogen overnight at room temperature. The molecular sieve was filtered, to this mixture was added saturated sodium bicarbonate, and was extracted 3 times with dichloromethane. Combined organic layer was dried over magnesium sulfate, filtered, and concentrated under vacuum. Gave 0.8 g of oil, which was purified using reverse phase HPLC C18 column, with a gradient of 20 to 90% acetonitrile-0.1% TFA, yield to 0.6 g of white powder. LC-MSD, m/z for: C₃₂H₃₈N₂O₃ [M+H]: 499.4. LC retention time on HPLC, C18 column gradient 20-95% acetonitrile with 0.1% TFA in 7 minutes: 3.87 min.

Step 5: N-(S)-(1-Cyclohexylmethyl-pyrrolidin-2-ylmethyl)-3,4-dimethoxy-N-naphthalen-2-ylmethyl-benzamide.

N-(S)-(1-Cyclohex-3-enylmethyl-pyrrolidin-2-ylmethyl)-3,4-dimethoxy-N-naphthalen-2-ylmethyl-benzamide was dissolved in 5 mL methanol, to this solution was added 2 mg palladium 5% on carbon. The mixture was stirred under hydrogen at room temperature, under atmospheric pressure. After 2 hours the reaction goes to completion. The catalyst was filtered, methanol concentrated under vacuum, yield to 10 mg of white powder. LC-MSD, m/z for: C₃₂H₄₀N₂O₃ [M+H]: 501.4. LC retention time on HPLC, C18 column gradient 20-95% acetonitrile with 0.1% TFA in 7 minutes: 4.52 min.

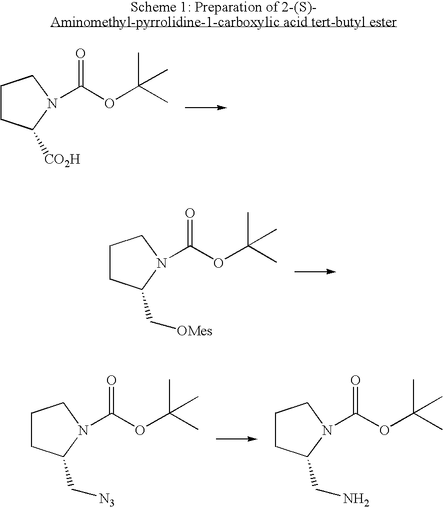

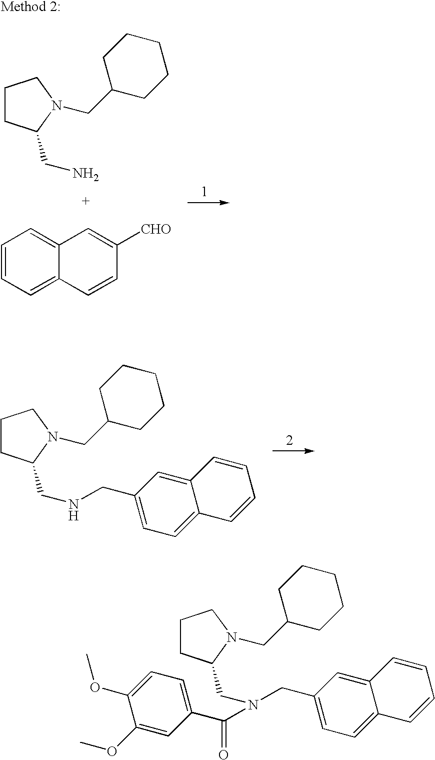

Step 1: (S)-(1′-Cyclohexylmethyl-pyrrolidin-2-ylmethyl)-naphthalen-2-ylmethyl-amine (S)—C-(1-Cyclohexylmethyl-pyrrolidin-2-yl)-methylamine (prepared according to scheme 2) 0.24 g (1 mmol), and naphthalene-2-carbaldehyde 0.19 g (1.2 mmol), were dissolved in 10 mL dichloromethane. To this mixture was added sodium triacethoxyborohydride 0.51 g (2 mmol), and molecular sieve. The reaction was stirred overnight under nitrogen. Molecular sieve was filtered, washed with 3 mL HCl, acidic layer was transformed to basic PH, with powder sodium bicarbonate, and extracted with ethyl acetate. The combined organic layer dried over magnesium sulfate, filtered and concentrated, yield to 100 mg of yellow oil.

Step 2: N-(S)-(1-Cyclohexylmethyl-pyrrolidin-2-ylmethyl)-3,4-dimethoxy-N-naphthalen-2-ylmethyl-benzamide.

Prepared according step 2 of method 1, from 3,4-dimethoxy benzoic acid 40 mg (0.22 mmol), 1-ethyl-3-(3-dimethylaminopropyl) carbodiimide hydrochloride 40 mg (0.22 mmol), 1-hydroxybenzotriazole 20 mg (0.18 mmol), triethylamine 0.03 mL (0.22 mmol) and (S)— (1-cyclohexylmethyl-pyrrolidin-2-yhnethyl)-naphthalen-2-ylmethyl-amine 50 mg (0.15 mmol), in 1 mL tetrahydrofuran. yield to 72 mg of white powder. LC-MSD, m/z for: C₃₂H₄₀N₂O₃ [M+H]: 501.4. LC retention time on HPLC, C18 column gradient 20-95% acetonitrile with 0.1% TFA in 7 minutes: 4.2 min.

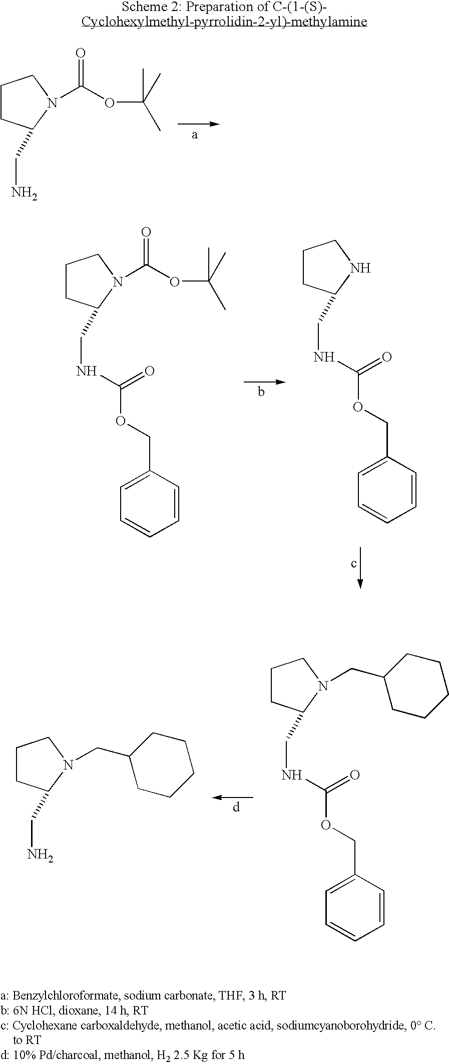

### Example 9

This example illustrates the preparation of N-(S)-(1-Cyclohexylmethyl-pyrrolidin-2-ylmethyl)-3,4-dimethoxy-N-quinolin-3-ylmethyl-benzamide.

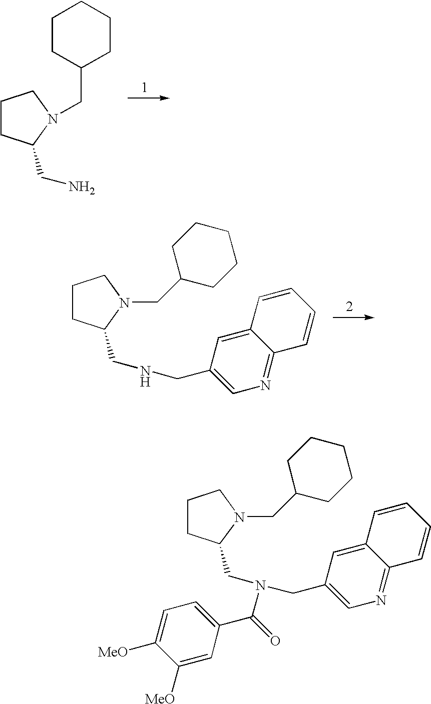

Step 1: (S)-(1-Cyclohexylmethyl-pyrrolidin-2-ylmethyl)-quinolin-3-ylmethyl-amine:

Experimental condition analogous to Example 8, from (S)—C-(1-cyclohexylmethyl-pyrrolidin-2-yl)-methylamine 0.25 g (1.3 mmol), quinoline-3-carbaldehyde 0.2 g (1.3 mmol), sodium triacethoxyborohydride 0.53 g (2.6 mmol), and molecular sieve in 8 mL dichloromethane. Yield to 40 mg of compound.

Step 2: N-(S)-(1-Cyclohexylmethyl-pyrrolidin-2-ylmethyl)-3,4-dimethoxy-N-quinolin-3-ylmethyl-benzamide

Experimental condition analogous to Example 8, from 3,4-dimethoxy benzoic acid 32 mg (0.17 mmol), 1-ethyl-3-(3-dimethylaminopropyl) carbodiimide hydrochloride 34 mg (0.17 mmol), 1-hydroxybenzotriazole 19 mg (0.14 mmol), triethylamine 0.24 mL and (S)-(1-cyclohexylmethyl-pyrrolidin-2-ylmethyl)-quinolin-3-ylmethyl-amine 40 mg (0.11 mmol) in 1 mL of tetrahydrofuran, yield after reverse phase HPLC purification with a C 18 column, gradient of 20-80% acetonitrile −0.1% TFA, gave 11 mg white solid. LC-MSD, m/z for: C₃₁H₃₉N₃O₃ [M+H]: 502.2. LC retention time on HPLC, C18 column gradient 20-95% acetonitrile with 0.1% TFA in 7 minutes: 3.2 min.

### Example 10

This example illustrates the preparation of N-benzofuran-2-ylmethyl-N-(S)-(1-cyclohexylmethyl-pyrrolidin-2-ylmethyl)-3,4-dimethoxy-benzamide.

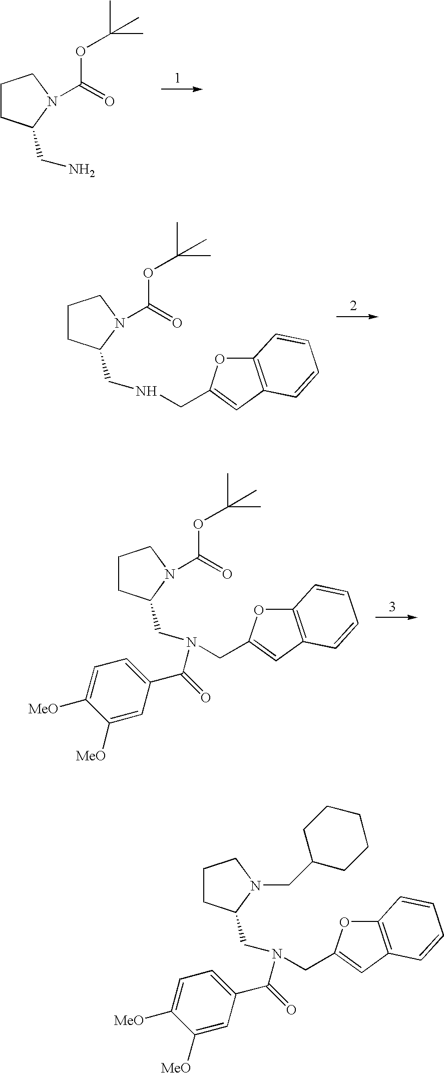

Step 1: 2-(S)-{[(Benzofuran-2-ylmethyl)-amino]-methyl}-pyrrolidin-1-carboxylic acid tert-butyl ester:

Experimental condition analogous to Example 8, from benzofuran-2-carbaldehyde 0.15 g (1 mmol), 2-aminomethyl-pyrrolidin-1-carboxylic acid tert-butyl ester 0.26 g (1.2 mmol), and sodium triacethoxyborohydride 0.43 g (2 mmol), in 10 mL dichloromethane, yield to 0.2 g of oil 88% pure.

Step 2: 2-(S)-{[Benzofuran-2-ylmethyl-(3,4-dimethoxy-benzoyl)-amino]-methyl}-pyrrolidine-1-carboxylic acid tert-butyl ester

Experimental condition analogous to Example 8, from 3,4,5-trimethoxy benzoic acid 72 mg (0.39 mmol), 1-ethyl-3-(3-dimethylaminopropyl) carbodiimide hydrochloride 75 mg (0.39 mmol), 1-hydroxybenzotriazole 45 mg (0.33 mmol), triethylamine 0.05 mL (0.39 mmol), and 2-(S)-{[(benzofuran-2-ylmethyl)-amino]-methyl}-pyrrolidine-1-carboxylic acid tert-butyl ester, 100 mg (0.3 mmol) in 3 mL tetrahydrofuran.

The compound was purified through silica gel chromatography elution with ethyl acetate: methanol 9:1, gave 93 mg of white oily compound.

Step 3: N-Benzofuran-2-ylmethyl-N-(S)-(1-cyclohexylmethyl-pyrrolidin-2-ylmethyl)-3,4-dimethoxy-benzamide

Experimental condition analogous to Example 8, from 2-(S)-{[benzofuran-2-ylmethyl-(3,4-dimethoxy-benzoyl)-amino]-methyl}-pyrrolidin-1-carboxylic acid tert-butyl ester 110 mg (0.22 mmol), and 1 mL of mixture of trifluoroacetic acid and dichloromethane 17%, after deprotection 64 mg (0.16 mmol) of the N-benzofuran-2-ylmethyl-3,4-dimethoxy-N-(S)-pyrrolidin-2-ylmethyl-benzamide, cyclohexanecarbaldehyde 19 mg (0.17 mmol), sodium triacethoxyborohydride 68 mg (0.32 mmol) and molecular sieve, in 1 mL dichloromethane. Yield to 50 mg of white powder. LC-MSD, m/z for: C₃₀H₃₈N₂O₄ [M+H]: 491.2. LC retention time on HPLC, C18 column gradient 20-95% acetonitrile with 0.1% TFA in 7 minutes: 3.91 min.

### Example 11

This example illustrates the preparation of N-Benzofuran-2-ylmethyl-N—(S)-(1-cyclohexylmethyl-pyrrolidin-2-ylmethyl)-3,4,5-trimethoxy-benzamide.

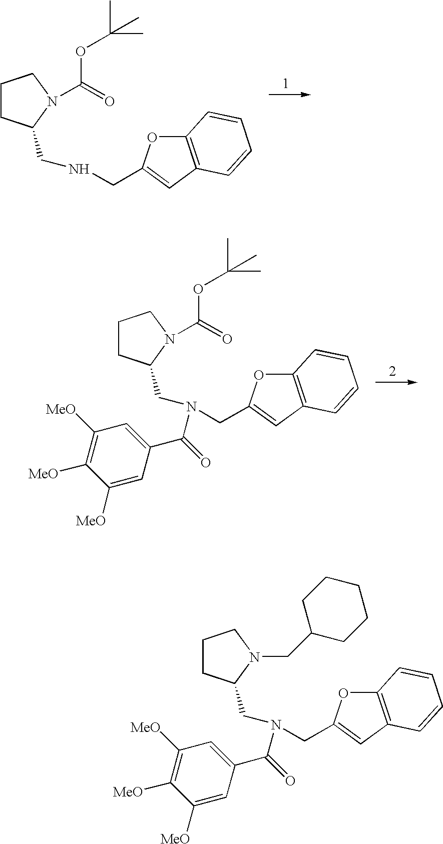

Step 1: 2-(S)-{[Benzofuran-2-ylmethyl-(3,4,5-trimethoxy-benzoyl)-amino]-methyl}-pyrrolidin-1-carboxylic acid tert-butyl ester.

2-(S)-{[Benzofuran-2-ylmethyl]-amino]-methyl}-pyrrolidin-1-carboxylic acid tert-butyl ester 0.33 g (0.28 mmol), 3,4,5-trimethoxy-benzoyl chloride 65 mg (0.28 mmol) and triethylamine 0.04 mL (0.28 mmol). After 1 hour, saturated sodium bicarbonate added and the mixture extracted with dichloromethane, combined organic layer, dried over magnesium sulfate, filtered, and concentrated under vacuum. Purification over silica gel chromatography, elution ethyl acetate-hexane 5.5-4.5, gave 100 mg of light yellow oil.

Step 2: N-Benzofuran-2-ylmethyl-N-(S)-(1-cyclohexylmethyl-pyrrolidin-2-ylmethyl)-3,4,5-trimethoxy-benzamide.

Experimental condition analogous to example 10, from of —(S)-{[benzofuran-2-ylmethyl-(3,4,5-trimethoxy-benzoyl)-amino]-methyl}-pyrrolidin-1-carboxylic acid tert-butyl ester 0.1 g (0.19 mmol), 0.15 mL (1.9 mmol) trifluoroacetic acid, in 1 mL dichloromethane. The deprotected amine 0.06 g (0.188 mmol) was added to cyclohexanecarbaldehyde 0.023 g (0.19 mmol), and sodium acethoxy borohydride 0.06 g (0.38 mmol) in 1 mL of dichloromethane, yield 70 mg of white powder. LC-MSD, m/z for: C₃₁H₄₀N₂O₅ [M+H]: 521.2. LC retention time on HPLC, C18 column gradient 20-95% acetonitrile with 0.1% TFA in 7 minutes: 3.88 min.

### Example 12

This example illustrates the preparation of 3,4,5-Trimethoxy-N-[2-(1-methyl-pyrrolidin-2-yl)-ethyl]-N-naphthalen-2ylmethyl-benzamide.

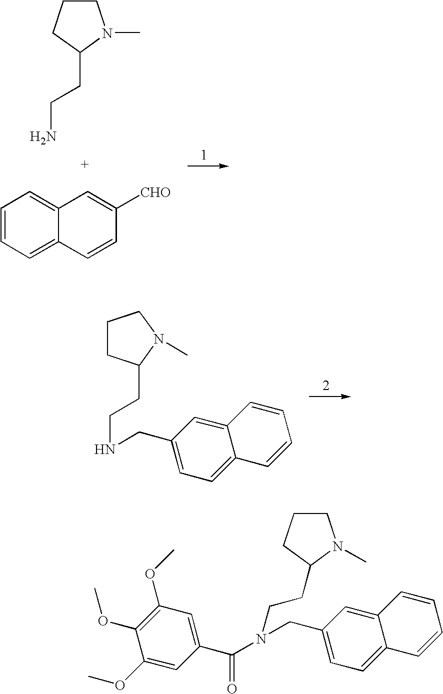

Step 1: [2-(1-Methyl-pyrrolidin-2-yl)-ethyl]-naphthalen-2-ylmethyl-amine.

Experimental condition analogous to Example 8, from 2-naphthalencarboxaldehyde 0.15 g (1 mmol), 2-(1-methyl-pyrrolidin-2-yl)-ethylamine 0.14 g (1.1 mmol), and sodium triacethoxyborohydride 0.31 g (1.5 mmol), in 10 mL dichloromethane. The crude material is 110 mg pale yellow oil.

Step 2: 3,4,5-Trimethoxy-N-[2-(1-methyl-pyrrolidin-2-yl)-ethyl]-N-naphthalen-2ylmethyl-benzamide.

Experimental condition analogous to Example 11, from [2-(1-methyl-pyrrolidin-2-yl)-ethyl]-naphthalen-2-ylmethyl-amine 0.11 g (0.42 mmol), 3,4,5-trimethoxy-benzoylchloride 0.11 g (0.51 mmol), and triethylamine 0.06 g (0.72 mmol), in 10 mL of anhydrous dichloromethane. The compound was purified using reverse phase HPLC, C18 column gradient of 20-80% acetonitrile-0.1% TFA, yield to 180 mg of pure material. LC-MSD, m/z for: C₂₈H₃₄N₂O₄ [M+H]: 463.5. LC retention time on HPLC, C18 column gradient 20-95% acetonitrile with 0.1% TFA in 7 minutes: 3.01 min.

### Example 13

This example illustrates the preparation of 3,4,5-Trimethoxy-N[2-(1-methyl-pyrrolidin-2-yl)-ethyl]-N-naphthalen-1-ylmethyl-benzamide.

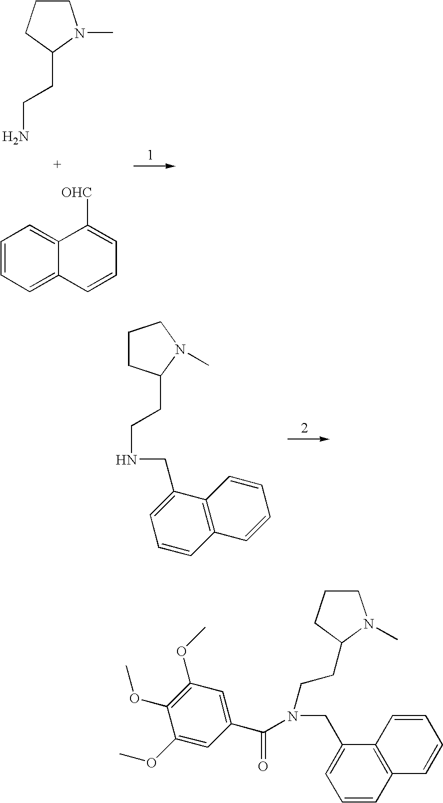

Step 1: [2-(1-Methyl-pyrrolidin-2-yl)-ethyl]-naphthalen-1-ylmethyl-amine.

Experimental condition analogous to Example 12, from 1-naphthalencarboxaldehyde 0.15 g (1 mmol), 2-(1-methyl-pyrrolidin-2-yl)-ethylamine 0.14 g (1.1 mmol), and sodium triacethoxyborohydride 0.31 g (1.5 mmol), in 10 mL dichloromethane. The crude material is 210 mg clear oil.

Step 2: 3,4,5-Trimethoxy-N[2-(1-methyl-pyrrolidin-2-yl)-ethyl]-N-naphthalen-1-ylmethyl-benzamide.

Experimental condition analogous to Example 12, from [2-(1-Methyl-pyrrolidin-2-yl)-ethyl]-naphthalen-1-ylmethyl-amine 0.21 g (0.79 mmol), 3,4,5-trimethoxy-benzoylchloride 0.0.21 g (0.95 mmol), and triethylamine 0.11 g (1.18 mmol), in 15 mL of anhydrous dichloromethane. The compound was purified using reverse phase HPLC, C18 column with a gradient of 20-80% acetonitrile-0.1% TFA, yield to 280 mg of pure material. LC-MS1), m/z for: C₂₈H₃₄N₂O₄ [M+H]: 463.5. LC retention time on HPLC, C18 column gradient of 20-95% acetonitrile with 0.1% TFA in 7 minutes: 3.13 min.

### Example 14

This example illustrates the preparation of N-Benzofuran-2-ylmethyl-3,4,5-trimethoxy-N-[2-(1-methyl-pyrrolidin-2-yl)-ethyl]-benzamide.

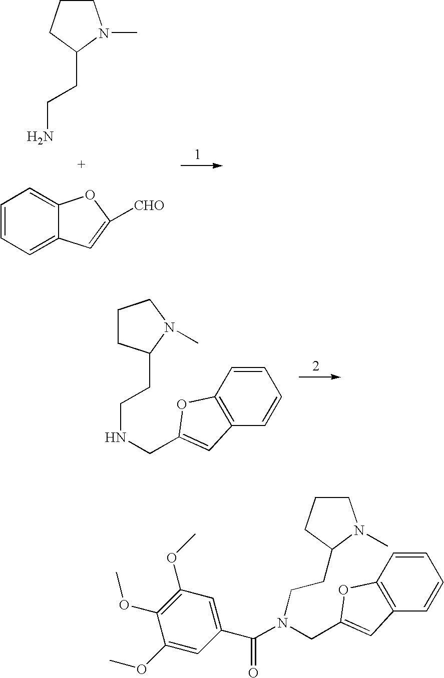

Step 1: Benzofuran-2ylmethyl-[2-(1-methyl-pyrrolidin-2-yl)-ethyl]-amine.

Experimental condition analogous to Example 12, from 2-benzofurancarboxaldehyde 0.14 g (1.1 mmol), 2-(1-methyl-pyrrolidin-2-yl)-ethylamine 0.14 g (1 mmol), and sodium triacethoxyborohydride 0.31 g (1.5 mmol), in 10 mL Dichloromethane. Purification using silica chromatography, elution with dichloromethane-methanol-ammonium hydroxide, 9-1-0.25, gave 83 mg of compound.

Step 2: N-Benzofuran-2-ylmethyl-3,4,5-trimethoxy-N-[2-(1-methyl-pyrrolidin-2-yl)-ethyl]-benzamide.

Experimental condition analogous to Example 8, from 3,4,5-trimethoxy benzoic acid 0.08 g (0.32 mmol), 1-ethyl-3-(3-dimethylaminopropyl) carbodiimide hydrochloride 0.060 g (0.38 mmol), 1-hydroxybenzotriazole 0.05 g (0.38 mmol), triethylamine 0.05 mL (0.38 mmol), and benzofuran-2ylmethyl-[2-(1-methyl-pyrrolidin-2-yl)-ethyl]-amine 0.08 g (0.3 mmol) in 3 mL tetrahydrofuran. Purification using reverse phase HPLC, C18 column with a gradient of 20-80% acetonitrile −0.1% TFA, gave 100 mg of white powder as a TFA salt. LC-MSD, m/z for: C₂₆H₃₂N₂O₅ [M+H]: 453.5. LC retention time on HPLC, C18 column gradient 20-95% acetonitrile with 0.1% TFA in 7 minutes: 2.73.

### Example 15

This example illustrates the preparation of N-Benzo[b]thiophen-2-ylmethyl-3,4,5-trimethoxy-N-[2-(1-methyl-pyrrolidin-2-yl)-ethyl]-benzamide.

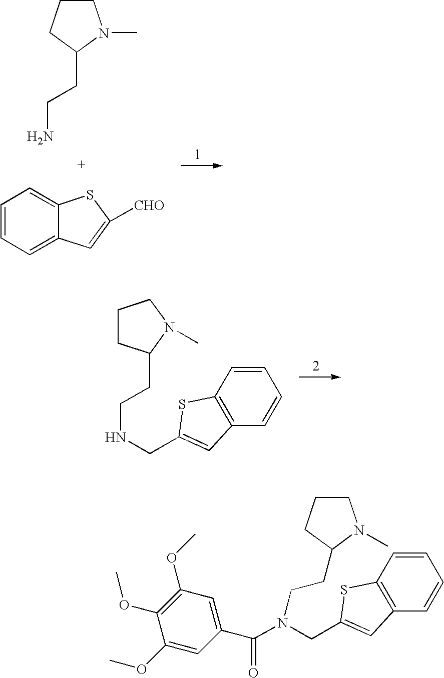

Step 1: Benzo[b]thiophen-2-ylmethyl-[2-(1-methyl-pyrrolidin-2-yl)-ethyl]-amine.

Experimental condition analogous to Example 12, from benzo[b]thiophen-2-carbaldehyde 0.18 g (1.1 mmol), 2-(1-methyl-pyrrolidin-2-yl)-ethylamine 0.14 g (1 mmol), and sodium triacethoxyborohydride 0.31 g (1.5 mmol), in 10 mL dichloromethane. Purification using silica gel chromatography, elution with dichloromethane-methanol-ammonium hydroxide, 9-1-0.25, gave 73 mg of compound.

Step 2: N-Benzo[b]thiophen-2-ylmethyl-3,4,5-trimethoxy-N-[2-(1-methyl-pyrrolidin-2-yl)-ethyl]-benzamide.

Experimental condition analogous to Example 8, from 3,4,5-dimethoxy benzoic acid 0.07 g (0.26 mmol), 1-ethyl-3-(3-dimethylaminopropyl) carbodiimide hydrochloride 0.05 g (0.31 mmol), 1-hydroxybenzotriazole 0.04 g (0.3 mmol), triethylamine 0.03 mL (0.31 mmol), and benzo[b]thiophen-2-ylmethyl-[2-(1-methyl-pyrrolidin-2-yl)-ethyl]-amine 0.07 g (0.26 mmol) in 3 mL tetrahydrofuran. Purification using reverse phase HPLC, C18 column with a gradient 20-80% acetonitrile −0.1% TFA, gave 50 mg of hydroscopic powder, as a TFA salt. LC-MSD, m/z for: C₂₆H₃₂N₂O₄S [M+H]: 469.5. LC retention time on HPLC, C18 column gradient 20-95% acetonitrile with 0.1% TFA in 7 minutes: 2.858.

### Example 16

This example illustrates the preparation of N-(2,3-Dihydro-benzo[1,4]dioxin-6-ylmethyl)-3,4,5-trimethoxy-N-[2-(1-methyl-pyrrolidin-2-yl)-ethyl]-benzamide.

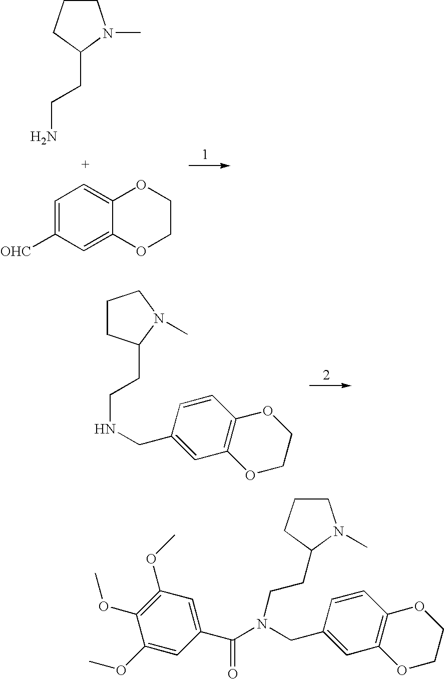

Step 1: (2,3-Dihydro-benzo[1,4]dioxin-6-ylmethyl)-[2-(1-methyl-pyrrolidin-2-yl)-ethyl]-amine.

Experimental condition analogous to Example 12, from benzo[b]thiophen-2-carbaldehyde 0.18 g (1.1 mmol), 2-(1-methyl-pyrrolidin-2-yl)-ethylamine 0.14 g (1 mmol), and sodium triacethoxyborohydride 0.31 g (1.5 mmol), in 10 mL dichloromethane. Purification using silica gel chromatography, elution with dichloromethane-methanol-ammonium hydroxide, 9-1-0.25, gave 0.21 g of compound.

Step 2: (2,3-Dihydro-benzo[1,4]dioxin-6-ylmethyl)-[2-(1-methyl-pyrrolidin-2-yl)-ethyl]-amine.

Experimental condition analogous to Example 8, from 3,4,5-dimethoxy benzoic acid 0.19 g (0.91 mmol), 1-ethyl-3-(3-dimethylaminopropyl) carbodiimide hydrochloride 0.14 g (0.91 mmol), 1-hydroxybenzotriazole 0.12 g (0.91 mmol), triethylamine 0.12 mL (0.91 mmol), and (2,3-dihydro-benzo[1,4]dioxin-6-ylmethyl)-[2-(1-methyl-pyrrolidin-2-yl)-ethyl]-amine 0.21 g (0.76 mmol) in 5 mL tetrahydrofuran. Purification using reverse phase HPLC, C18 column with a gradient of 20-80% acetonitrile −0.1% TFA, gave 150 mg compound as a TFA salt. LC-MSD, m/z for: C₂₆H₃₄N₂O₆ [M+H]: 471.5. LC retention time on HPLC, C18 column gradient 20-95% acetonitrile with 0.1% TFA in 7 minutes: 1.805.

### Example 17

This example illustrates the preparation of 3,4,5-Trimethoxy-N-[2-(1-methyl-pyrrolidin-2yl)-N-quinolin-3-ylmethyl-benzamide.

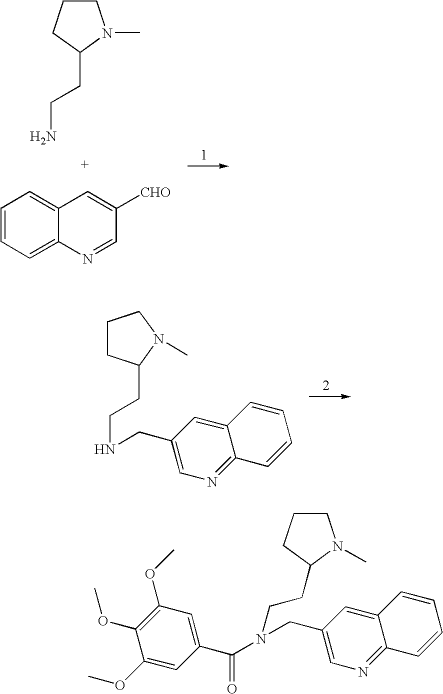

Step 1: [2-(1-Methyl-pyrrolidin-2-yl)-ethyl-quinolin-3-ylmethyl-amine.

Experimental condition analogous to Example 12, from quinoline-3-carbaldehyde 0.25 g (1.5 mmol), 2-(1-methyl-pyrrolidin-2-yl)-ethylamine 0.22 g (1.8 mmol), and sodium triacethoxyborohydride 0.31 g (1.5 mmol), in 10 mL dichloromethane. Purification using silica gel chromatography, elution with dichloromethane-methanol-ammonium hydroxide, 9-1-0.25, gave 160 mg of light yellow oily compound.

Step 2: 3,4,5-Trimethoxy-N-[2-(1-methyl-pyrrolidin-2yl)-N-quinolin-3-ylmethyl-benzamide.

Experimental condition analogous to Example 8, from 3,4,5-dimethoxy benzoic acid 0.11 g (0.55 mmol), 1-ethyl-3-(3-dimethylaminopropyl) carbodiimide hydrochloride 0.10 g (0.55 mmol), 1-hydroxybenzotriazole 0.05 g (0.40 mmol), triethylamine 0.08 mL (0.55 mmol), and [2-(1-Methyl-pyrrolidin-2-yl)-ethyl-quinolin-3-ylmethyl-amine 0.1 g (0.37 mmol) in 5 mL tetrahydrofuran. Purification using silica gel chromatography elution using dichloromethane-methanol: 9-1 gave 80 mg light yellow oil. LC-MSD, m/z for: C₂₇H₃₃N₃O₄ [M+H]: 464.5. LC retention time on HPLC, C18 column gradient 20-95% acetonitrile with 0.1% TFA in 7 minutes: 1.93 min.

### Example 18

This example illustrates the preparation of 3,4,5-Trimethoxy-N-[2-(1-methyl-pyrrolidin-2yl)-N-quinolin-2-ylmethyl-benzamide.

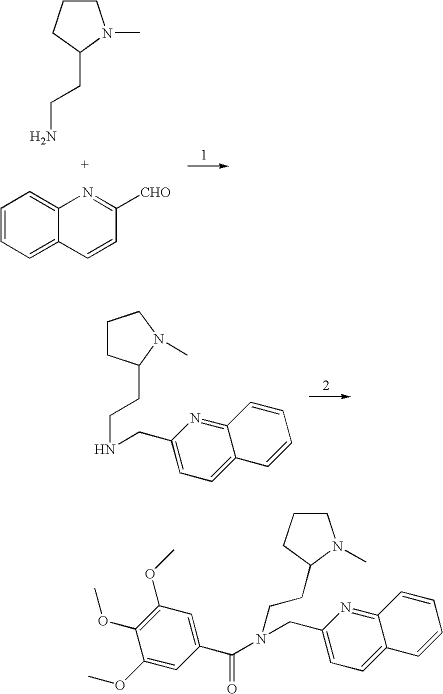

Step 1: [2-(1-Methyl-pyrrolidin-2-yl)-ethyl-quinolin-2-ylmethyl-amine.

Experimental condition analogous to Example 12, from quinoline-2-carbaldehyde 0.25 g (1.5 mmol), 2-(1-methyl-pyrrolidin-2-yl)-ethylamine 0.22 g (1.8 mmol), and sodium triacethoxyborohydride 0.31 g (1.5 mmol), in 10 mL dichloromethane. Purification using silica chromatography, elution with dichloromethane-methanol-ammonium hydroxide, 9-1-0.25, gave 0.24 g of dark orange oily compound.

Step 2: 3,4,5-Trimethoxy-N-[2-(1-methyl-pyrrolidin-2yl)-N-quinolin-2-ylmethyl-benzamide.

Experimental condition analogous to Example 8, from 3,4,5-dimethoxy benzoic acid 0.11 g (0.55 mmol), 1-ethyl-3-(3-dimethylaminopropyl) carbodiimide hydrochloride 0.10 g (0.55 mmol), 1-hydroxybenzotriazole 0.05 g (0.40 mmol), triethylamine 0.08 mL (0.55 mmol), and [2-(1-methyl-pyrrolidin-2-yl)-ethyl-quinolin-2-ylmethyl-amine 0.1 g (0.37 mmol) in 5 mL tetrahydrofuran. Purification using silica gel chromatography elution using dichloromethane-methanol: 9-1 gave 50 mg light yellow oil. LC-MSD, m/z for C₂₇H₃₃N₃O₄ [M+H]: 464.5. LC retention time on HPLC, C18 column gradient 20-95% acetonitrile with 0.1% TFA in 7 minutes: 0.81.

### Example 19

This example illustrates the preparation of N-Benzo[b]thiophen-3-ylmethyl-3,4,5-trimethoxy-N-[2-(1-methyl-pyrrolidin-2-yl)-ethyl]-benzamide.

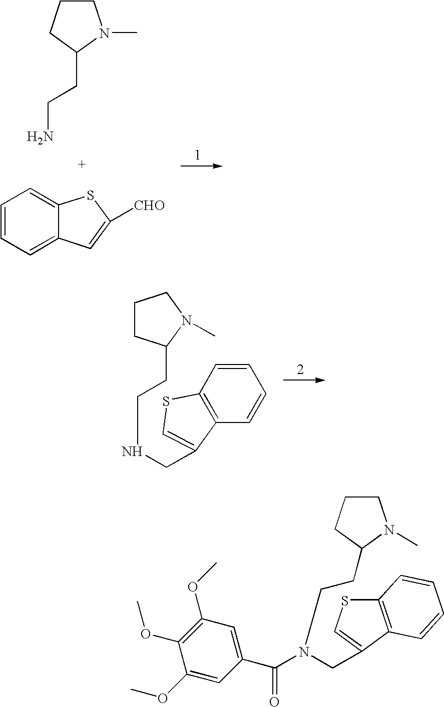

Step 1: Benzo[b]thiophen-3-ylmethyl-[2-(1-methyl-pyrrolidin-2-yl)-ethyl]-amine.

Experimental condition analogous to Example 12, from benzo[b]thiophen-3-carbaldehyde 0.16 g (1 mmol), 2-(1-methyl-pyrrolidin-2-yl)-ethylamine 0.14 g (1.1 mmol), and sodium triacethoxyborohydride 0.31 g (1.5 mmol), in 10 mL Dichloromethane. Purification using silica chromatography, elution with dichloromethane-methanol-ammonium hydroxide, 9-1-0.25, gave 140 mg of compound.

Step 2: N-Benzo[b]thiophen-3-ylmethyl-3,4,5-trimethoxy-N-[2-(1-methyl-pyrrolidin-2-yl)-ethyl]-benzamide.

Experimental condition analogous to Example 8, from 3,4,5-dimethoxy benzoic acid 0.13 g (0.62 mmol), 1-ethyl-3-(3-dimethylaminopropyl) carbodiimide hydrochloride 0.09 g (0.62 mmol), 1-hydroxybenzotriazole 0.08 g (0.62 mmol), triethylamine 0.08 mL (0.62 mmol), and benzo[b]thiophen-2-ylmethyl-[2-(1-methyl-pyrrolidin-2-yl)-ethyl]-amine 0.14 g (0.51 mmol) in 3 mL tetrahydrofuran. Purification using reverse phase HPLC C18 column with a gradient of 20-80% acetonitrile −0.1% TFA, gave 120 mg of hydroscopic powder, as a TFA salt. LC-MSD, m/z for: C₂₆H₃₂N₂O₄S [M+H]: 469.5. LC retention time on HPLC, C18 column gradient 20-95% acetonitrile with 0.1% TFA in 7 minutes: 3.07 min.

### Example 20

This example illustrates the preparation of N-Benzothiazol-2-ylmethyl-3,4,5-trimethoxy-N-[2-(1-methyl-pyrrolidin-2-yl)-ethyl]-benzamide.

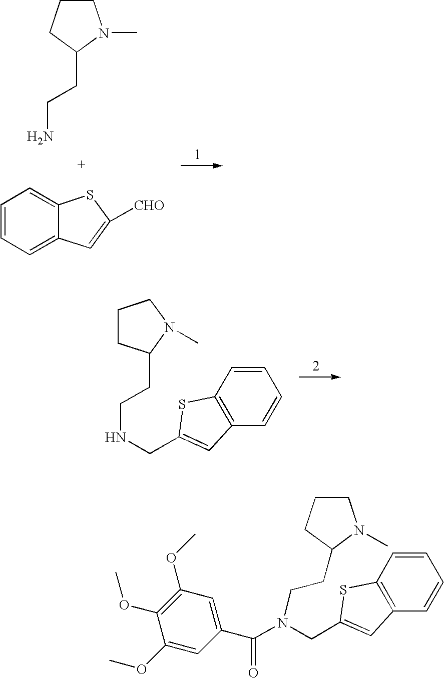

Step 1: Benzo[thiazol-2-ylmethyl-[2-(1-methyl-pyrrolidin-2-yl)-ethyl]-amine.

Experimental condition analogous to Example 12, from benzothiazol-2-carbaldehyde 0.16 g (1 mmol), 2-(1-methyl-pyrrolidin-2-yl)-ethylamine 0.14 g (1.1 mmol), and sodium triacethoxyborohydride 0.31 g (1.5 mmol), in 10 mL dichloromethane. Purification using silica gel chromatography, elution with dichloromethane-methanol-ammonium hydroxide, 9-1-0.25, gave 0.15 mg of compound.

Step 2: N-Benzotriazol-2-ylmethyl-3,4,5-trimethoxy-N-[2-(1-methyl-pyrrolidin-2-yl)-ethyl]-benzamide.

Experimental condition analogous to Example 8, from 3,4,5-dimethoxy benzoic acid 0.14 g (0.65 mmol), 1-ethyl-3-(3-dimethylaminopropyl) carbodiimide hydrochloride 0.1 g (0.65 mmol), 1-hydroxybenzotriazole 0.08 g (0.65 mmol), triethylamine 0.09 mL (0.65 mmol), and benzotriazole-2-ylmethyl-[2-(1-methyl-pyrrolidin-2-yl)-ethyl]-amine 0.15 g (0.54 mmol) in 3 mL tetrahydrofuran. Purification using reverse phase HPLC, C18 column gradient of 20-80% acetonitrile −0.1% TFA, gave 120 mg of hydroscopic powder, as a TFA salt. LC-MSD, m/z for: C₂₅H₃₁N₃O₄S [M+H]: 470.5. LC retention time on HPLC, C18 column gradient 20-95% acetonitrile with 0.1% TFA in 7 minutes: 2.50.

### Example 21

This example illustrates the preparation of 3,4,5-Trimethoxy-N-(1-methyl-1H-benzoimidazol-2-ylmethyl)-N-[2-(1-methyl-pyrrolidin-2-yl)-ethyl]-benzamide.

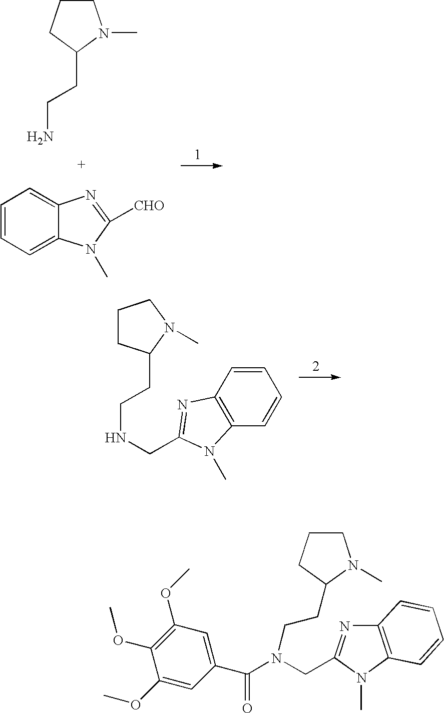

Step 1: (1-Methyl-1H-benzoimidazol-2-ylmethyl)-[2-(1-methyl-pyrrolidin-2-yl)-ethyl]-amine.

Experimental condition analogous to Example 12, from 1-methyl-1H-benzoimidazol-2-carbaldehyde 0.2 g (1.25 mmol), 2-(1-methyl-pyrrolidin-2-yl)-ethylamine 0.18 g (1.38 mmol), and sodium triacethoxyborohydride 0.39 g (1.87 mmol), in 10 mL dichloromethane. After work-up the material was used as a crude.

Step 2: 3,4,5-Trimethoxy-N-(1-methyl-1H-indol-2-ylmethyl)-N-[2-(1-methyl-pyrrolidin-2-yl)-ethyl]-benzamide.

Experimental condition analogous to Example 12, from (1-methyl-1H-benzoimidazol-2-ylmethyl)-[2-(1-methyl-pyrrolidin-2-yl)-ethyl]-amine the crude, 3,4,5-trimethoxy-benzoylchloride 0.37 g (1.62 mmol), and triethylamine 0.26 mL (1.87 mmol), in 5 mL of anhydrous dichloromethane. The compound was purified using reverse phase HPLC, C18 column with a gradient of 20-80% acetonitrile-0.1% TFA, yield to 110 mg of pure material. LC-MSD, m/z for: C₂₆H₃₄N₄O₄ [M+H]: 467.2. LC retention time on HPLC, C18 column gradient 20-95% acetonitrile with 0.1% TFA in 7 minutes: 0.43 min.

### Example 22

This example illustrates the preparation of N-(1H-Indol-2ylmethyl)-3,4,5-trimethoxy-N-[2-(1-methyl-pyrrolidin-2-yl)-ethyl]-benzamide.

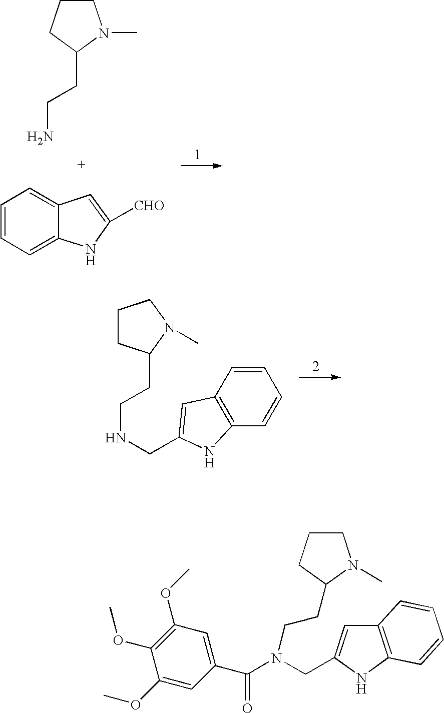

Step 1: (1H-Indol-2ylmethyl)-[2-(1-methyl-pyrrolidin-2-yl)-ethyl]-amine.

Experimental condition analogous to Example 12, from 1H-indole-2-carbaldehyde 0.14 g (2 mmol), 2-(1-methyl-pyrrolidin-2-yl)-ethylamine 0.3 g (2.4 mmol), and sodium triacethoxyborohydride 0.87 g (1.87 mmol), in 20 mL Dichloromethane. The compound was purified using silica gel chromatography elution, ethyl-acetate-methanol-amonium hydroxide: 9-1-0.1 to 8-2-0.2, yield to 0.3 g light brown oil.

Step 2: N-(1H-Indol-2ylmethyl)-3,4,5-trimethoxy-N-[2-(1-methyl-pyrrolidin-2-yl)-ethyl]-benzamide

Experimental condition analogous to Example 12, from (1H-indol-2ylmethyl)-[2-(1-methyl-pyrrolidin-2-yl)-ethyl]-amine 0.8 g (0.31 mmol), 3,4,5-trimethoxy-benzoylchloride 0.08 g (0.34 mmol), and triethylamine 0.06 mL (0.46 mmol), in 5 mL of anhydrous dichloromethane. The compound was purified using reverse phase HPLC, C18 column with a gradient of 20-70% acetonitrile-0.1% TFA, yield to 50 mg of pure material. LC-MSD, m/z for: C₂₆H₃₃N₃O₄ [M+H]: 452.2. LC retention time on HPLC, C18 column gradient 20-95% acetonitrile with 0.1% TFA in 7 minutes: 1.99 min.

### Example 23

This example illustrates the preparation of N-(1H-Indol-2ylmethyl)-3,5-dimethoxy-N-[2-(1-methyl-ppyrrolidin-2-yl)-ethyl]-benzamide.

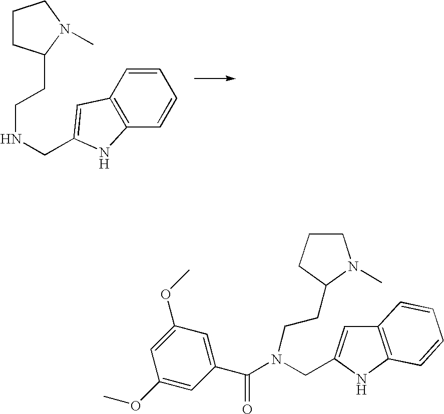

Experimental condition analogous to Example 12, from (1H-indol-2ylmethyl)-[2-(1-methyl-pyrrolidin-2-yl)-ethyl]-amine 0.1 g (0.38 mmol), 3,5-dimethoxy-benzoylchloride 0.08 g (0.42 mmol), and triethylamine 0.08 mL (0.57 mmol), in 1.5 mL of anhydrous dichloromethane. The compound was purified using reverse phase HPLC, C18 column with a gradient of 20-70% acetonitrile-0.1% TFA, yield to 50 mg of pure material. LC-MSD, m/z for: C₂₅H₃₁N₃O₃ [M+H]: 422.2. LC retention time on HPLC, C18 column gradient 20-95% acetonitrile with 0.1% TFA in 7 minutes: 2.7 min.

### Example 24

This example illustrates the preparation of N-Biphenyl-3-yl-3,4,5-trimethoxy-N-[3-(2-methyl-piperidin-1-yl)-propyl]-benzamide.

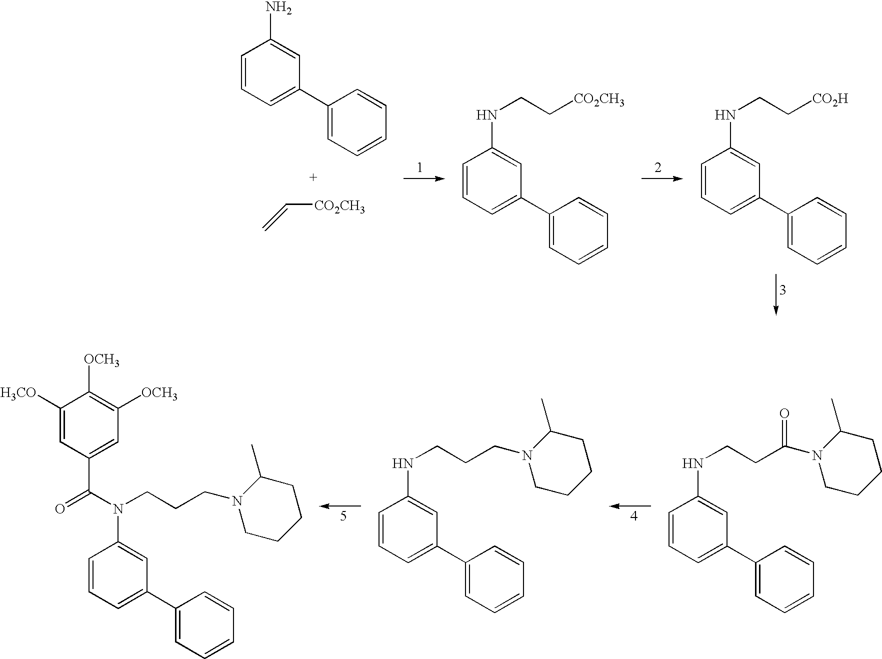

Step 1: 3-(Biphenyl-3-ylamino)-propionic acid methyl ester.

In a round bottom flask was added 3-aminobiphenyl 2.6 g (15.3 mmol), methyl acrylate 1.5 g (16.9 mmol) and cupric acetate 0.1 g, the reaction mixture was stirred 5 hour at 90° C., another 5 equivalent of methyl acrylate 7 g (75 mmol), and 0.25 g of cupric acetate was added, and the reaction mixture was heated for another 5 hours. The crude was purified using silica gel chromatography using 15% of ethyl acetate and petroleum ether. Yield to 1.6 g of oil.

Step 2: 3-(Biphenyl-3-ylamino)-propionic acid.

3-(Biphenyl-3-ylamino)-propionic acid methyl ester 1.6 g (6.3 mmol) was taken in 8 mL of water and 8 mL of tetrahydrofuran, to this solution was added 0.4 g (9.5 mmol) of lithium hydroxide, reaction stirred at room temperature for 5 hour. The solvent was removed from the mixture completely and 10 mL water was added and washed with ethyl acetate. The aqueous solution was acidified with 1 M HCl, and was extracted with ethyl acetate 3 times. Combined organic layer was washed with brine, dried over magnesium sulfate and concentrated under vaccum, yield to 1.6 g of acid used as crude for the next step.

Step 3: 3-(Biphenyl-3-ylamino)-1-(2-methyl-piperidin-1-yl)-propan-1-one.

To a mixture of the 3-(biphenyl-3-ylamino)-propionic acid 1.6 g (6.6 mmol), 2-methylpiperidine 0.78 g (7.9 mmol), was added the solid O-(benzotriazole-1-yl)-N,N,N′,N′-tetramethyluronium tetrafluoroborate 4.7 g (1.3 mmol), and triethylamine 3.86 mL (27 mmol) in 25 mL of dichloromethane and left overnight at room temperature. The reaction mixture was washed with water, the organic layer, dried over magnesium sulfate, filtered, and concentrated under vacuum. The compound was purified using silica gel chromatography and was eluted with ethyl acetate, yield to 2 g material.

Step 4: Biphenyl-3-yl-[3-(2-methyl-piperdin-1-yl)-propyl]-amine.

3-(Biphenyl-3-ylamino)-1-(2-methyl-piperidin-1-yl)-propan-1-one 1 g (3.1 mmol) in 10 mL of tetrahydrofuran was added dropwise to a cold solution of lithium aluminium hydride 0.1 g (3.1 mmol) in 10 mL dry tetrahydrofuran. The mixture was stirred for 5 hour then was quenched with saturated solution of sodium sulfate. The compound was purified by silica gel chromatography using chloroform-methanol 9:1, yield to 0.2 g of compound.

Step 5: N-Biphenyl-3-yl-3,4,5-trimethoxy-N-[3-(2-methyl-piperidin-1-yl)-propyl]-benzamide.

3,4,5-trimethoxybenzoic acid 0.19 g (0.89 mmol) was dissolved in thionyl chloride 0.26 mL (3.5 mmol) and refluxed for 3 hours under a guard tube. The excess of thionyl chloride was removed under vacuum. Biphenyl-3-yl-[3-(2-methyl-piperdin-1-yl)-propyl]-amine 0.23 g (0.746 mmol) was taken in 5 mL dichloromethane, triethylamine 4.1 mL (3 mmol) was then added, the mixture was then cooled and acid chloride in 5 mL dichloromethane was added dropwise and was stirred overnight. The solvent was removed under vacuum and the compound was purified by silica gel chromatography using chloroform-methanol 9-1, gave 40 mg of compound. LC-MSD, m/z for: C₃₁H₃₈N₂O₄ [M+H]: 503.6. LC retention time on HPLC, C18 column gradient 20-95% acetonitrile with 0.1% TFA in 7 minutes: 3.96.

### Example 24

This example illustrates the preparation of 3,4,5-Trimethoxy-N-[3-(2-methyl-piperidin-1-yl)-N-naphthalen-2-ylmethyl-benzamide.

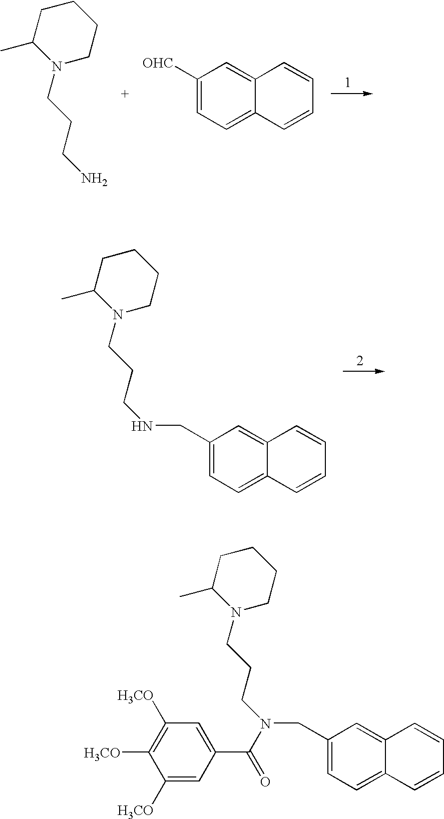

Step 1: [3-(2-Methyl-piperidin-1-yl)-propyl]-naphthalen-2-ylmethyl-amine.

2-Naphtaldehyde 1 g (6.4 mmol), 3-(2-methyl-piperidin-1-yl)-propylamine 0.99 g (6.4 mmol), in 25 mL of dry dichloromethane was added 5 g of molecular sieve. The reaction was stirred overnight at room temperature. The molecular sieve was filtered and dichloromethane was concentrated under vacuum. To the mixture was added 15 mL of dry methanol and sodium borohydride 0.3 g (8 mmol) after 30 minutes reaction goes to completion, methanol was concentrated under vacuum, and was diluted with chloroform, organic layer was washed with 2 times 20 mL water, followed with brine. The organic layer was dried over magnesium sulfate and concentrated under vacuum. The compound was purified using silica gel chromatography elution, with 3.5% methanol in chloroform, yield 0.6 g oil.

Step 2: 3,4,5-Trimethoxy-N-[3-(2-methyl-piperidin-1-yl)-N-naphthalen-2-ylmethyl-benzamide.

[3-(2-Methyl-piperidin-1-yl)-propyl]-naphthalen-2-ylmethyl-amine 0.55 g (1.8 mmol), 3,4,5-trimethoxy-benzoic acid 0.04 g (2.2 mmol), triethylamine 0.02 mL and O-(benzotriazol-1-yl)-N,N,N′,N′-tetramethyluronium tetrafluoroborate 0.18 g (3.6 mmol), 5 mL of anhydrous dichloromethane. The compound was purified using 2% methanol in chloroform. LC-MSD, m/z for: C₃₀H₃₈N₂O₄ [M+H]: 491.6. LC retention time on HPLC, C18 column gradient 20-95% acetonitrile with 0.1% TFA in 7 minutes: 3.86 min.

### Example 25

This example illustrates the preparation of 3,4,5-Trimethoxy-N-[3-(2-methyl-piperidin-1-yl)-propyl]-N-(5-phenyl-thiazol-2-ylmethyl)-benzamide.

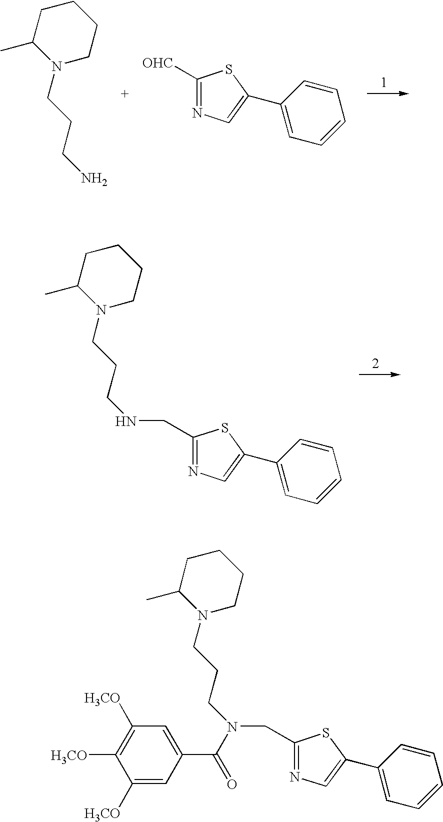

Step 1: [3-(2-Methyl-piperidin-1-yl)-propyl]-(5-phenyl-thiazol-2ylmethyl)-amine.

Experimental condition analogous to Example 24, from 5-phenyl-thiazole-2-carbaldehyde 0.16 g (1.05 mmol), 3-(2-methyl-piperidin-1-yl)-propylamine 0.2 g (1.05 mmol), in 5 mL of dry dichloromethane was added 2 g of molecular sieve. The reaction was stirred overnight at room temperature. The molecular sieve was filtered and dichloromethane was concentrated under vaccum. To the mixture was added 15 mL of dry methanol and sodium borohydride 0.04 g (1.155 mmol) was added at 0° C. after 30 minutes reaction goes to completion. The reaction was quenched with 2 mL acetone, methanol was concentrated under vacuum, and was diluted with chloroform, organic layer was washed with 2 times 20 mL water, followed with brine. The organic layer was dried over magnesium sulfate and concentrated under vaccum. Yield 0.3 g of compound.

Step 2: 3,4,5-Trimethoxy-N-[3-(2-methyl-piperidin-1-yl)-propyl]-N-(5-phenyl-thiazol-2-ylmethyl)-benzamide.

Experimental condition analogous to Example 24, from [3-(2-methyl-piperidin-1-yl)-propyl]-(5-phenyl-thiazol-2ylmethyl)-amine 0.15 g (0.45 mmol), 3,4,5-trimethoxy-benzoic acid 0.10 g (0.499 mmol), triethylamine 0.15 mL and 1-propanephosphonic acid cyclic anhydride (50% in ethyl acetate) 0.34 g (0.54 mmol) 20 mL of ethyl acetate. The compound was purified using neutral alumina gel chromatography elution with chloroform, gave 120 mg of material. LC-MSD, m/z for: C₂₉H₃₇N₃O₄S [M+H]: 524.6. LC retention time on HPLC, C18 column gradient 20-95% acetonitrile with 0.1% TFA in 7 minutes: 3.54.

### Example 26

This example illustrates the preparation of 3,4,5-Trimethoxy-N-[3-(2-methyl-piperidin-1-yl)-propyl]-N-naphthalen-2-yl-benzamide.

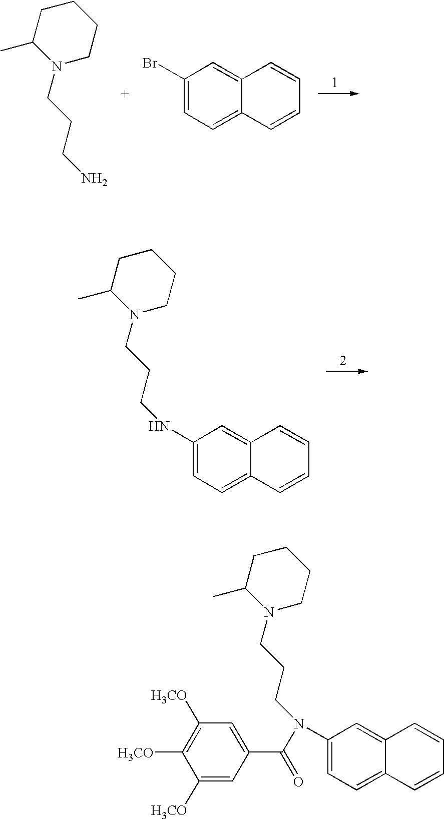

Step 1: [3-(2-Methyl-piperidin-1-yl)-propyl]-naphthalen-2-yl amine.

In round bottom flask under nitrogen, was added palladium (II) acetate 0.09 g (0.4 mmol), rac-2,2′-bis(diphenylphosphino)-1,1′-binaphtyl 0.53 g (0.8 mmol), tripotassium phospate mono basic 0.06 g (29 mmol), in 25 mL DME, to this mixture was added 2-bromonaphthalene 1.7 g (8.2 mmol), and 2-methyl-piperidine-N-propylamine 4 g (25.6 mmol). The mixture was refluxed 17 hours. The reaction mixture was filtered through celite and concentrated. The compound was purified using silica gel chromatography, elution with 5% methanol in chloroform. Yield to 0.47 g of compound.

Step 2: 3,4,5-Trimethoxy-N-[3-(2-methyl-piperidin-1-yl)-propyl]-N-naphthalen-2-yl-benzamide.

Experimental condition analogous to Example 24 from, 3,4,5 trimethoxy benzoic acid 0.53 g (2.5 mmol), thionyl chloride 0.24 mL (3.34 mmol), triethylamine 0.7 mL (5 mmol) and [3-(2-methyl-piperidin-1-yl)-propyl]-naphthalen-2-yl amine 0.47 g (1.67 mmol) in 15 mL chloroform. The compound was purified using silica gel chromatography gave 150 mg of material. LC-MSD, m/z for: C₂₉H₃₆N₂O₄ [M+H]: 477.5. LC retention time on HPLC, C18 column gradient 20-95% acetonitrile with 0.1% TFA in 7 minutes: 3.49.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1. A method of modulating angiogenesis in a subject, the method comprising administering to the subject an agent that modulates CCX-CKR2 activity.

2. The method of claim 1, wherein the agent modulates binding of a ligand to CCX-CKR2.

3. The method of claim 2, wherein the ligand is selected from the group consisting of SDF-1 and I-TAC.

4. The method of claim 1, wherein the method promotes CCX-CKR2 activity, thereby promoting angiogenesis.

5. The method of claim 4, wherein the agent is administered in combination with a second agent that promotes angiogenesis.

6. The method of claim 1, wherein the agent is a CCX-CKR2 agonist.

7. The method of claim 6, wherein the agonist is selected from a polypeptide, an antibody and an agent with a mass of less than 1,500 daltons.

8. The method of claim 4, wherein the CCX-CKR2 activity is promoted by expressing recombinant CCX-CK2 in a cell of the subject.

9. The method of claim 8, wherein the cell is an endothelial cell.

10. The method of claim 4, wherein the CCX-CKR2 activity is promoted by administering I-TAC to the subject.

11. The method of claim 10, wherein I-TAC is administered locally to the subject.

12. The method of claim 4, wherein the subject is in need of increased vascularization.

13. The method of claim 1, wherein the subject has a wound, fracture, burn, inflammatory disease, heart disease, restinosis, ischeric heart, peripheral vascular disease, myocardial infarction, stroke, infertility, psoriasis or scleroderma.

14. The method of claim 13, wherein the subject has a wound and the agent is applied to the wound, thereby enhancing wound healing.

15. The method of claim 14, wherein the agent inhibits CCX-CKR2 activity.

16. The method of claim 15, wherein the agent is a polynucleotide that inhibits expression of CCX-CKR2.

17. The method of claim 15, wherein the agent is an antagonist selected from the group consisting of a polypeptide, an antibody and an agent with a mass of less than 1,500 daltons.

18. The method of claim 14, wherein the agent enhances CCX-CKR2 activity.

19. The method of claim 1, wherein the method decreases CCX-CKR2 activity, thereby reducing angiogenesis.

20. The method of claim 19, wherein the agent is a polynucleotide that inhibits expression of CCX-CKR2.

21. The method of claim 19, wherein the agent is administered in combination with a second anti-angiogenic agent.

22. The method of claim 1, wherein the agent is a CCX-CKR2 antagonist.

23. The method of claim 22, wherein the antagonist is selected from a polypeptide, an antibody and an agent with a mass of less than 1,500 daltons.

24. The method of claim 19, wherein the subject has cancer.

25. The method of claim 24, wherein the subject has a solid tumor and the agent is targeted or delivered to the tumor.

26. The method of claim 24, wherein an amount of a chemotherapeutic agent or radiation is administered to the subject in combination with the agent.

27. The method of claim 26, wherein the amount is sub-therapeutic when the chemotherapeutic agent or radiation is administered alone.

28. The method of claim 19, wherein the subject does not have cancer.

29. The method of claim 19, wherein angiogenesis is reduced in a tissue selected from an eye, skin, joint, ovarian tissue or endometrial tissue.

30. The method of claim 19, wherein the agent is used as a birth control agent.

31. The method of claim 1, wherein the subject has arthritis, and the agent is administered in an amount effective to reduce arthritis symptoms in the subject.

32. A pharmaceutical composition comprising an amount of a chemotherapeutic agent in combination with an agent that decreases CCX-CKR2 activity.

33. The pharmaceutical composition of claim 32, wherein the amount is sub-therapeutic when the chemotherapeutic agent is administered alone.

34. A pharmaceutical composition comprising an agent that increases CCX-CKR2 activity and a second agent that promotes angiogenesis.

35. A pharmaceutical composition comprising an agent that decreases CCX-CKR2 activity and a second agent that decreases angiogenesis.

36. A pharmaceutical composition comprising an agent that decreases CCX-CKR2 activity and a second anti-arthritis agent.
